(19) United States
(12) Patent Application Publication
(10) Pub. No.: US 2026/0144805 A1
(43) Pub. Date: May 28, 2026

(54) CANNABIDIOL COMPOSITIONS FOR USE IN THE TREATMENT OF NEURODEGENERATIVE AND NEUROLOGICAL DISORDERS

(71) Applicant: JAZZ PHARMACEUTICALS RESEARCH UK LIMITED, Kent (GB)

(72) Inventors: Benjamin WHALLEY, Kent (GB); Sally LOOMIS, Kent (GB); Alan SILCOCK, Kent (GB); Amy FISHER, Kent (GB); Elena SAMOYLENKO, Kent (GB); Jennifer LI, Kent (GB); David VIRLEY, Kent (GB); Andrew MCCREARY, Kent (GB)

(21) Appl. No.: 19/102,763

(22) PCT Filed: Aug. 11, 2023
(86) PCT No.: PCT/EP2023/072328

§ 371 Date: Feb. 10, 2025

(30) Foreign Application Priority Data
Aug. 11, 2022 (GB) 2211753.5
Aug. 11, 2022 (GB) 2211755.0

(51) Int. Cl. A61K 31/00; A61K 45/06; A61P 25/00; A61P 25/14; A61P 25/22; A61P 25/28

(52) U.S. Cl. CPC A61K 31/658; A61K 45/06; A61P 25/00; A61P 25/14; A61P 25/22; A61P 25/28

(57) ABSTRACT

The present disclosure provides cannabinoid drug substances, pharmaceutical compositions and methods of treating neurological or neurodegenerative disorders in a human patient.

### CROSS-REFERENCE TO RELATED APPLICATIONS

The present application claims priority to Provisional Application Nos. GB2211755.0 and GB2211753.5, the contents of which are hereby incorporated by reference in their entireties for all purposes.

### BACKGROUND

Disorders of the nervous system are diverse and include a wide range of disorders, diseases, and pathological conditions affecting the brain, encompassing both neurodevelopmental and neurodegenerative disorders.

Neurodegenerative diseases are a group of disorders characterised by changes in normal neuronal functioning, leading, in most cases, to neuronal death. Most of these diseases are associated, especially in late stages, with severe neuronal loss. Neurodegeneration can be described as the progressive damage or death of neurones whose primary function is to assist in the memory process. The damage or death of neurones leads to a gradual deterioration of the functions controlled by the affected part of the nervous system. With an ever-increasing ageing population, progressively more individuals are affected by neurodegenerative diseases.

One of the most common neurodegenerative disorders is Alzheimer's Disease (AD), itself the most common type of dementia.

Alzheimer's Disease (AD) is an age-associated degenerative disorder of the brain. It is neuropathologically characterised by the aberrant aggregation of amyloid-β protein into extracellular plaques as well as hyperphosphorylated tau protein into intraneuronal neurofibrillary tangles and dystrophic neurites surrounding the plaques, which represent the hallmarks of the disease. Clinically, AD is characterized by slow and gradual progressive cognitive deterioration, typically starting in the domains of learning and memory, and executive functioning. As the disease progresses, patients ultimately reach the dementia stage in which they lose their ability to perform normal activities of daily living and require full-time care and institutionalization. The 2018 World Alzheimer's Disease report estimated that 50 million people worldwide suffered from dementia and that the number of cases would triple by 2050. AD emerges as the most prevalent cause of dementia, accounting for 50-60% of all cases.

Alongside progressive loss of cognition and function, dementia presents another important challenge, collectively referred to as behavioural and psychological symptoms of dementia (BPSD). BPSD represent a heterogeneous group of non-cognitive symptoms and behaviours occurring in patients with dementia. BPSD include psychosis, agitation, aggression, irritability, depression, and anxiety. The aetiology of dementia-related psychosis and agitation/aggression is very complex and is often a cluster of biological factors (anatomical and neurochemical changes) as well as psychological and social aspects (responses to stress, living arrangements).

Agitation is seen in 20 to 50% of those with moderate-to-severe AD and is one of the most prominent neuropsychiatric symptoms with high prevalence and persistence. It adds greatly to the public health burden of AD and is associated with decreased quality of life (QOL), increased caregiver burden, and higher rates of institutionalization and mortality.

The International Psychogeriatric Association Agitation Definition Work Group's definition of agitation in cognitive disorders highlights core behaviours of agitation associated with evidence of emotional distress (e.g., rapid changes in mood, irritability, outbursts).

Not only are there no drug treatments approved for treatment of Agitation associated with Alzheimer's Disease (AiAD), the standard of care (atypical antipsychotics) carries a “black box” warning placed by the FDA for increased mortality in this population as studies have linked some of these to a higher risk of death for people with dementia, with also a very poor side effect profile that can diminish compliance and adherence. Further, atypical antipsychotics, although used, do not work for all patients.

Another progressive neurodegenerative disorder is Essential Tremor (ET) which is the most common movement disorder, with global prevalence being approximately 1%. The disorder presents mainly in the hands and arms as an action tremor as opposed to resting tremor. Additional symptoms may include head tremor (e.g., a “yes” or “no” motion) without abnormal posturing of the head and a shaking or quivering sound to the voice. Approximately half of cases are caused by genetic risk factors (familial tremor), and the gene HS1-BP3 accounts for 10% of cases.

Pathophysiology and underlying neurochemical mechanisms of ET are poorly understood.

While the pathophysiological mechanism of ET is unclear, drugs that reduce neuronal membrane excitability are beneficial in ET. For example, propranolol is a commonly used beta-blocker that reduces membrane excitability and is used to treat ET. However, side effects of such beta blockers may include fatigue, light-headedness, or heart problems, and as such these may not be an option for patients with asthma or certain heart problems. Other medications that might be prescribed include anti-seizure medication such as gabapentin and topiramate. Topiramate has a GABA-mimetic effect and inhibits glutamatergic neurotransmission. Side effects include drowsiness and nausea.

Neurodevelopmental disorders are a group of conditions with onset in the developmental period. The disorders typically manifest early in development, often before the child reaches school-age, and are characterized by developmental deficits that produce impairments of personal, social, academic, or occupational functioning. The range of developmental deficits varies from very specific limitations of learning or control of executive functions to global impairments of social skills or intelligence.

Genetics can play an important role in many neurodevelopmental disorders, and some cases of certain conditions such as intellectual disability are associated with specific genes. However, most neurodevelopmental disorders have complex and multiple contributors rather than any one clear cause. These disorders likely result from a combination of genetic, biological, psychosocial and environmental risk factors.

Autism Spectrum Disorders (ASD) is a lifelong neurodevelopmental disorder characterised by core symptoms of restricted, repetitive patterns of behaviour and persistent deficits in social communication and interaction. In the United States alone it is estimated that more than 5 million (>2.21%) adults have ASD. Associated signs and symptoms can include intellectual disability, language impairment, motor abnormalities (e.g., motor delay, hypotonia, catatonia and deficits in coordination), gastrointestinal problems, sleep disorders, psychiatric comorbidities (most commonly anxiety and depression), and behavioural dysregulation such as aggression, impulsivity, irritability, and self-injurious behaviour. Behaviour dysregulation is among the most commonly occurring conditions. In a Food and Drug Administration Patient (FDA) Focused Drug Development Initiative (FDA 2018), behavioural dysregulation, anxiety, and depression were identified by individuals and caregivers as key concerns. Behavioural issues can significantly impact the ability to care for patients at home.

ASD research has largely been focused on children; however, as prevalence increases and children transition into adulthood, the impact of ASD in adulthood must be addressed. A review of health records for a population of adults with autism in the US, indicated that adults with autism had significantly increased rates of all major psychiatric disorders including depression, anxiety, bipolar disorder, obsessive-compulsive disorder, schizophrenia, and suicide attempts. Nearly all medical conditions were significantly more common in adults with autism, including immune conditions, gastrointestinal and sleep disorders, seizure, obesity, dyslipidemia, hypertension, and diabetes.

Longitudinal and cross-sectional studies indicate the majority of autistic adults struggle with achieving independent living, employment, maintaining friendships, managing co-occurring mental health conditions, and have poorer quality of life. Higher costs are associated with caring for adults with ASD than children, mostly driven by residential care or supportive living accommodation and individual productivity.

Although some improvements may be seen over time, outcomes do not necessarily improve. Simonoff et al. (2020) found scores in social responsiveness scale to be stable over time, in contrast with some improvement in IQ, suggesting no improvement in ASD symptomology. Howlin et al. (2013) reported some improvement in symptom severity over time, however social outcomes did not improve. Anderson et al. (2011) reported an increase in social withdrawal between the ages of 9 and 18, despite general improvement in other maladaptive behaviours. Taken together, ASD symptoms and impacts persist through adult life.

ASD patients present symptoms in a heterogenous way:
Core symptoms across patients include manifestations of social communication impairment and repetitive and restrictive behaviour.
Non-core symptoms are exhibited by a significant proportion (65-80%) of patients, including behavioural issues (aggression, irritability), hyperactivity, intellectual deficit, sleep disorders, epilepsy (paediatric), and depression (more common in adults).

The term irritability is commonly used in ASD to describe a wider range of challenging behaviours that also include aggression towards others, deliberate self-injuriousness, temper tantrums, and quickly changing moods. Irritability associated with ASD is a commonly occurring symptom (30-60% of patients with ASD), which puts a tremendous daily burden on their caregivers.

Irritability in ASD has mostly been investigated in children; however, it is an issue relevant into adulthood, especially as older and more physically mature adults can cause more harm to themselves or others around them when the irritability manifests as physical aggression.

A longitudinal study on a sample of 74 adults indicated that although irritability behaviours tend to decrease in adulthood, they are still present in adulthood with 20% of the sample displaying physical aggression, 28% self-injurious behaviour, and 18% property destruction. In a sample of 500 ASD adults referred to a regional autism centre in Italy, 20% had co-occurring challenging behaviours.

In a sample of 126 adolescents and adults with autism (minimum age 15), aggressive behaviours were reported for 31% of participants; aggressive behaviours were associated with an increased use of medications, with 67% using atypical antipsychotics.

In another longitudinal study which examined maladaptive behaviours in a sample of 92 children with ASD from the age of 9 to the age of 18, overall irritability reduced over time. However, the subgroup with higher irritability scores at the age of 9 (16% of the sample), continued to display high irritability scores at the age of 18, highlighting the pervasive nature of these behaviours.

To date, two drugs, risperidone and aripiprazole, are approved for the treatment of irritability associated with ASD in children and adolescents aged 6 to 17 years. There are currently no treatments approved for the treatment of irritability in adults with ASD. However atypical antipsychotics are often prescribed to adults for irritability despite significant adverse effects such as over-sedation, metabolic, and extrapyramidal effects. A significant proportion of patients present drug-refractory aggression, self-injurious behaviour, and severe tantrums: in a sample of 94 people with ASD aged 2 to 54 years who reported aggression, self-injurious behaviours, and severe tantrums, 40% presented these behaviours despite previous treatment trials of both aripiprazole and risperidone (68% of cases) or of at least 3 psychoactive drugs targeting aggression, self-injurious behaviours, and severe tantrums, including either aripiprazole or risperidone (32% of cases).

There are no known efficacious treatments for the core social symptoms, although effects on repetitive behaviours are indicated with some data e.g., with the use of antidepressants and atypical antipsychotics. However, research on the treatment of core symptoms is beset by limitations in study design, statistical power and other issues inherent to the study of treatments for autism (e.g., heterogeneity of the disorder).

Clearly there is a significant need for effective therapies to prevent and treat such neurodegenerative or neurodevelopmental disorders.

### BRIEF SUMMARY OF THE DISCLOSURE

The present disclosure provides cannabinoid drug substances, pharmaceutical compositions, and methods of treating neurological or neurodegenerative disorders in a human patient.

In embodiments, the present disclosure provides a drug substance, comprising: about 65% w/w to about 95% w/w of cannabidiol (CBD) based on the total weight of the drug substance; and one or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

In embodiments, the drug substance comprises about 0.4% w/w to about 3.0% w/w trans-THC. In embodiments, the drug substance comprises about 1.0% w/w to about 4.0% w/w CBC. In embodiments, the drug substance comprises about 0.2% w/w to about 4.0% w/w CBG. In embodiments, the drug substance comprises about 0.2% w/w to about 1.4% w/w CBDV. In embodiments, the drug substance comprises about 0.1% w/w to about 0.8% w/w CBD-C4. In embodiments, the drug substance comprises about 0.1% w/w to about 1.6% w/w cis-THC. In embodiments, the drug substance further comprises one or more terpenes, triglycerides, sterols, fatty acids, or carotenoids. In embodiments, the drug substance comprises about 1.0% w/w to about 3.0% w/w terpenes. In embodiments, the drug substance comprises about 0.8% w/w and 3.0% w/w triglycerides. In embodiments, the drug substance comprises about 0.5% w/w to about 2.0% w/w sterols. In embodiments, the drug substance comprises two or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC). In embodiments, the drug substance comprises three or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC). In embodiments, the drug substance comprises four or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC). In embodiments, the drug substance comprises five or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC). In embodiments, the drug substance comprises about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), and about 0.1% w/w to about 5.0% w/w of cis-tetrahydrocannabinol (cis-THC).

In embodiments, the present disclosure provides a pharmaceutical composition, comprising the drug substance of the present disclosure and a pharmaceutically acceptable excipient. In embodiments, the pharmaceutically acceptable excipient is a carrier, diluent, adjuvant, filler, buffer, binder, disintegrant, preservatives, antioxidant, lubricant, stabiliser, solubilizer, surfactant, masking agent, colouring agent, flavouring agent, or sweetening agent. In embodiments, the pharmaceutical composition is in a form of a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, or an aerosol.

In embodiments, the present disclosure provides a method of treating a neurological or neurodevelopmental disorder in a patient in need thereof, comprising administering the drug substance or the pharmaceutical composition of the present disclosure. In embodiments, the neurological disorder or neurodevelopmental disorder is autism spectrum disorder (ASD), Alzheimer's Disease (AD), sleep disturbance, or psychiatric disorder. In embodiments, the psychiatric disorder is psychosis depression, or schizophrenia.

In embodiments, the present disclosure provides a method of reducing symptoms associated with a neurological or neurodevelopmental disorder in a patient in need thereof, comprising administering the drug substance or the pharmaceutical composition of the present disclosure. In embodiments, the symptoms comprise deficits in social interaction, agitation, anxiety, repetitive behaviours, irritability, aggression, hyperactivity, or a combination thereof.

In embodiments, the present disclosure provides a method of treating essential tremor in a patient in need thereof, comprising administering the drug substance or the pharmaceutical composition of the present disclosure.

In embodiments, the present disclosure provides a composition characterised in that it comprises about 65 to about 95% (w/w) cannabidiol (CBD) and one or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC).

The composition of the first aspect may further comprise one or more of the following cannabinoids: cannabidiol-C1 (CBD-C1); hydroxy-cannabidiol (OH-CBD); cannabielsoin I (CBE I); and cannabinol (CBN).

The first composition of the first aspect may comprise about 70 to about 100% (w/w) total cannabinoids; or about 75 to about 90% (w/w) total cannabinoids. The composition may comprise about 75%; about 76%; about 77%; about 78%; about 79%; about 80%; about 81%; about 82%; about 83%; about 84%; about 85%; about 86%; about 87%; about 88%; about 89%; or about 90% (w/w) cannabinoids.

The composition of the first aspect may comprise about 65 to about 95% (w/w) CBD; about 65 to about 90% (w/w) CBD; about 65 to about 85% (w/w) CBD; about 70 to about 85% (w/w) CBD; or about 70 to about 80% (w/w) CBD. The composition may comprise about 70%; about 71%; about 72%; about 73%; about 74%; about 75%; about 76%; about 77%; about 78%; about 79%; about 80%; about 81%; about 82%; about 83%; about 84%; or about 85% (w/w) CBD.

The composition of the first aspect may comprise about 0.4 to about 3.0%; about 0.5 to about 2.5%; or about 1 to about 2% (w/w) trans-THC.

The composition of the first aspect may comprise about 1.0 to about 4.0%; about 1.2 to about 2.5%; or about 1.5 to about 2.0% (w/w) CBC.

The composition of the first aspect may comprise about 0.2 to about 4.0%; about 0.3 to about 1.5%; or about 0.5 to about 1.2% (w/w) CBG.

The composition of the first aspect may comprise about 0.2 to about 1.4%; about 0.2 to about 1.0%; or about 0.1 to about 0.8% (w/w) CBDV.

The composition of the first aspect may comprise about 0.1 to about 0.8%; about 0.1 to about 0.5%; or about 0.1 to about 0.4% (w/w) CBD-C4.

The composition of the first aspect may comprise about 0.1 to about 1.6%; about 0.2 to about 1.2%; or about 0.5 to about 1.2% (w/w) cis-THC.

The composition of the first aspect may comprise about 65 to about 95% (w/w) cannabidiol (CBD); about 0.4 to about 3.0% (w/w) trans-THC; about 1.0 to about 4.0% (w/w) CBC; about 0.2 to about 4.0% (w/w) CBG; about 0.2 to about 1.4% (w/w) CBDV; about 0.1 to about 0.8% (w/w) CBD-C4; and/or about 0.1 to about 1.6% (w/w) cis-THC.

The composition of the first aspect may comprise about 65 to about 85% (w/w) cannabidiol (CBD); about 0.5 to about 2.5% (w/w) trans-THC; about 1.2 to about 2.5% (w/w) CBC; about 0.3 to about 1.5% (w/w) CBG; about 0.2 to about 1.0% (w/w) CBDV; about 0.1 to about 0.5% (w/w) CBD-C4; and/or about 0.2 to about 1.2% (w/w) cis-THC.

The composition of the first aspect may comprise about 70 to about 80% (w/w) cannabidiol (CBD); about 1 to about 2% (w/w) trans-THC; about 1.5 to about 2.0% (w/w) CBC; about 0.5 to about 1.2% (w/w) CBG; about 0.1 to about 0.8% (w/w) CBDV; about 0.1 to about 0.4% (w/w) CBD-C4; and/or about 0.5 to about 1.2% (w/w) cis-THC.

The composition of the first aspect may comprise a ratio of between 30:1 CBD:THC and 60:1 CBD:THC; a ratio of between 35:1 CBD:THC and 55:1 CBD:THC; or a ratio of between 40:1 CBD:THC to 50:1 CBD:THC.

A portion of at least one of the cannabinoids present in the composition of the first aspect may be isolated from cannabis plant material. Alternatively, a portion of at least one of the cannabinoids present in the composition of the first aspect may be prepared synthetically.

The composition of the first aspect may comprise a non-cannabinoid component comprising one or more of: terpenes; triglycerides; sterols; fatty acids; and carotenoids. The terpenes may comprise trans-caryophyllene and/or alpha-bisabolol.

The composition of the first aspect may comprise about 1.0 to about 3.0% (w/w) terpenes; about 0.8 to about 3.0% (w/w) triglycerides; and/or about 0.5 to about 2.0% (w/w) sterols.

In a second aspect of the invention there is a pharmaceutical composition comprising the composition of the first aspect and one or more additional ingredients selected from carriers, diluents (e.g., oils), excipients, adjuvants, fillers, buffers, binders, disintegrants, preservatives, antioxidants, lubricants, stabilisers, solubilisers, surfactants, masking agents, colouring agents, flavouring agents, and sweetening agents.

The pharmaceutical composition of the second aspect is in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

In a third aspect of the present invention there is provided the composition of the first aspect, or the pharmaceutical composition of the second aspect, for use in a method of treatment.

The method of treatment in the third aspect is a method of treatment of a neurological disorder. The neurological disorder may be a neurodegenerative or neurodevelopmental disorder. The neurodegenerative or neurodevelopmental disorder may be Alzheimer's Disease (AD). Alternatively, the neurodegenerative disorder may be Essential Tremor (ET).

The method of treatment in the third aspect is a method of treatment of symptoms associated with a neurodevelopmental disorder, wherein the symptoms comprise one or more of the following: deficits in social interaction; repetitive behaviours; irritability; aggression; and hyperactivity.

The method of treatment in the third aspect is a method of treatment of core symptoms associated with ASD comprising one or more of: deficits in social interaction including deficits in exploratory behaviours, and repetitive behaviours.

The method of treatment in the third aspect is a method of treatment of non-core symptoms associated with ASD comprising one or more of: irritability, aggression, and hyperactivity.

The method of treatment in the third aspect is a method of treatment of adults with ASD, to treat one or more symptoms associated with ASD from the following group: deficits in social interaction; repetitive behaviours; irritability; aggression; and hyperactivity.

In a fourth aspect of the invention there is provided a method of treatment comprising administering to a patient in need of treatment a therapeutically effective amount of the compound of the compound of the first aspect or the pharmaceutical composition of the second aspect.

In a fifth aspect of the present invention there is provided a composition characterised in that it comprises about 30 to about 50% (w/w) cannabidiol (CBD) and one or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC).

The composition of the fifth aspect may comprise one or more of the following cannabinoids: cannabidiol-C1 (CBD-C1); hydroxy-cannabidiol (OH-CBD); cannabielsoin I (CBE I); and cannabinol (CBN).

The composition of the fifth aspect may comprise about 32.5 to about 47.5% (w/w) CBD; about 0.2 to about 1.5% (w/w) trans-THC; about 0.5 to about 2.0% (w/w) CBC; about 0.1 to about 2.0% (w/w) CBG; about 0.1 to about 0.7% (w/w) CBDV; about 0.05 to about 0.8% (w/w) CBD-C4; and/or about 0.05 to about 0.8% (w/w) cis-THC.

The composition of the fifth aspect may comprise about 32%; about 33%; about 34%; about 35%; about 36%; about 37%; about 38%; about 39%; about 40%; about 41%; (w/w); about 42%; about 43%; about 44%; about 45%; about 46%; about 47%; or about 48% (w/w) CBD.

The composition of the fifth aspect may comprise about 35 to about 50% (w/w) total cannabinoids; or about 38 to about 50% (w/w) total cannabinoids.

A portion of at least one of the cannabinoids present in the composition of the fifth aspect may be isolated from cannabis plant material. Alternatively, a portion of at least one of the cannabinoids present in the composition of the fifth aspect may be prepared synthetically.

The composition of the fifth aspect may comprise a non-cannabinoid component comprising one or more of: terpenes; triglycerides; sterols; fatty acids; and carotenoids. The terpenes may comprise trans-caryophyllene and/or alpha-bisabolol.

The composition of the fifth aspect may comprise about 0.5 to about 1.5% (w/w) terpenes; about 0.4 to about 1.5% (w/w) triglycerides; and/or about 0.25 to about 1.0% (w/w) sterols.

In a sixth aspect of the present invention there is provided the composition of the fifth aspect for use in a method of treatment.

The method of treatment in the sixth aspect is a method of treatment of a neurological disorder. The neurological disorder may be a neurodevelopmental disorder. The neurodevelopmental disorder may be Autism Spectrum Disorder (ASD).

The method of treatment in the sixth aspect is a method of treatment of symptoms associated with a neurodevelopmental disorder, wherein the symptoms comprise one or more of the following: deficits in social interaction; repetitive behaviours; irritability; aggression; and hyperactivity.

The method of treatment in the sixth aspect is a method of treatment of core symptoms associated with ASD comprising one or more of: deficits in social interaction including deficits in exploratory behaviours, and repetitive behaviours.

The method of treatment in the sixth aspect is a method of treatment of non-core symptoms associated with ASD comprising one or more of: irritability, aggression, and hyperactivity.

The method of treatment in the sixth aspect is a method of treatment of adults with ASD, in particular to treat one or more symptoms from the following group: deficits in social interaction; repetitive behaviours; irritability; aggression; and hyperactivity.

In a seventh aspect of the present invention there is provided a composition characterised in that it comprises about 60 to about 75% (w/w) cannabidiol (CBD) and one or more of the following cannabinoids: trans-tetrahydrocannabinol (trans-THC), cis-tetrahydrocannabinol (cis-THC); cannabidiolic acid (CBDA); cannabidivarin (CBDV); cannabidiol-C1 (CBD-C1); cannabidiol-C4 (CBD-C4); hydroxy-cannabidiol (OH-CBD); cannabielsoin I (CBE I); cannabielsoin II (CBE II); cannabigerol (CBG); cannabinol (CBN); cannabicyclol (CBL); cannabichromene (CBC); tetrahydrocannabivarin (THCV); and tetrahydrocannabinol-C4 (THC-C4).

The composition of the seventh aspect may comprise about 60 to about 75% (w/w) CBD; about 2 to about 3% (w/w) trans-THC; about 0.5 to about 1.5% (w/w) cis-THC; not more than 0.2% (w/w) CBDA; about 0.2 to about 1.0% (w/w) CBDV; not more than 0.2% (w/w) CBD-C1; about 0.1 to about 0.5% (w/w) CBD-C4; about 0.2 to about 1.0% (w/w) OH-CBD; not more than 0.3% (w/w) CBE I; not more than 0.1% (w/w) CBE II; about 0.5 to about 2.5% (w/w) CBG; not more than 0.2% (w/w) CBN; not more than 0.2% (w/w) CBL; about 1 to about 4% (w/w) CBC; not more than 0.1% (w/w) THCV; and/or not more than 0.1% (w/w) THC-C4. The composition of the seventh aspect may comprise about 65 to about 90% (w/w) total cannabinoids; about 70 to about 85% (w/w) total cannabinoids.

Certain aspects of this invention are further described, by way of example only.

### BRIEF DESCRIPTION OF THE DRAWINGS

Embodiments of the invention are further described hereinafter with reference to the accompanying drawings, in which:

FIGS. 1A-γ show the extraction processes of DS-A (FIG. 1A) and DS-β (FIG. 1B) as described in Example 1.

FIG. 2 shows the experimental design of the study of Example 2. Open field (OF) test measures locomoter activity. BW refers to body weight check.

FIGS. 3A-E show longitudinal assessment of Locomotor Activity in rTg4510 mice (FIG. 3A). FIGS. 3B-E are separate figures stratified by cohort, showing locomotor activity at weeks 21 and 26 in cohort 1 and cohort 2.

FIG. 4 shows the effect of Drug Substance (DS) on Locomotor Activity in rTg4510 mice at week 31, in 60-minute periods and total. Data are back-transformed means (n=15). Analysis was by robust regression with treatment, cohort and Day as factors and 0-60 minute activity on Wk 26 as a covariate. Groups A and C were excluded from the main analysis and analysis was repeated without the covariate but with Groups A and C included in order to perform the comparisons involving Wild-type and Doxycycline mice. Multiple comparisons were by multiple t test to compare rTg4510 to Wild-type, Doxycycline to Wild-type and rTg4510, and Risperidone to Wild-type and rTg4510, Dunnett's test to compare DS to Wild-type and Williams' test to compare DS to rTg4510. Significant differences vs Wild-type mice: * p<0.05, ** p<0.01. Significant differences vs rTg4510 mice: †p<0.05, ††p<0.01, †††p<0.001.

FIG. 5 shows the effect of DS on Locomotor Activity in rTg4510 mice at week 31 in 30-minute periods. Data are back-transformed means (n=15). Analysis was by robust regression with treatment, cohort and Day as factors and 0-60 minute activity on Wk 26 as a covariate. Groups A and C were excluded from the main analysis and analysis was repeated without the covariate but with Groups A and C included in order to perform the comparisons involving Wild-type and Doxycycline mice. Multiple comparisons were by multiple t test to compare rTg4510 to Wild-type, Doxycycline to Wild-type and rTg4510, and Risperidone to Wild-type and rTg4510, Dunnett's test to compare DS to Wild-type and Williams' test to compare DS to rTg4510. Significant differences vs Wild-type mice: * p<0.05, ** p<0.01. Significant differences vs rTg4510 mice: †p<0.05, ††p<0.01, †††p<0.001.

FIG. 6 shows effect of drug substance (30, 100 and 150 mg/kg) in a harmaline model of Essential Tremor, as carried out in Example 3. Statistical difference indicated by #p<0.05, ##p<0.01, ###p<0.001 veh/harmaline comparison or *** p<0.001 veh/veh comparison.

FIG. 7 shows the experimental design of the essential tremor study of Example 4. T=time (in minutes) relative to harmaline administration.

FIG. 8 shows the timeline of experiments carried out in Example 5.

FIGS. 9A-C show the effect of drug substance (DS) (10, 30 and 100 mg/kg) following 8 days drug treatment on irritability following in utero valproic acid exposure—effect on bottle brush reactivity in postnatal day 30 animals. Data represents mean±SEM number of aggressive behaviors (FIG. 9A), number of defensive behaviors (FIG. 9B) and irritability score (FIG. 9C) (sum of aggressive and defensive behaviors) over ten 10-second trials (n=8). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIGS. 10A-C show the effect of drug substance (10, 30 and 100 mg/kg) following 8 days drug treatment on reciprocal social behavior on postnatal day 30. Data represents mean±SEM latency to engage in social behavior (sec) (FIG. 10A), frequency of social behavior (a.u.) (FIG. 10B) and duration of social behavior (sec) (FIG. 10C) during a 15-minute trial (n=4). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIGS. 11A-C show the effect of drug substance (10, 30 and 100 mg/kg) following 8 days drug treatment on social deficit following in utero valproic acid exposure-effect on social play mounting behavior in postnatal day 30 animals. Data represents mean±SEM latency to engage in play (sec) (FIG. 11A), frequency of bouts of play behavior (a.u.) (FIG. 11B) and total time playing (sec) (FIG. 11C) during a 15-minute trial (n=4). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, or ####p<0.0001.

FIGS. 12A-C show the effect of drug substance (10, 30 and 100 mg/kg) following 8 days drug treatment on social deficit following in utero valproic acid exposure-effect on social play pinning behavior in postnatal day 30 animals. Data represents mean±SEM latency to engage in pinning (sec) (FIG. 12A), frequency of bouts of pinning behavior (a.u.) (FIG. 12B) and total time pinning (sec) (FIG. 12C) during a 15-minute trial (n=4). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIGS. 13A-C show the effect of drug substance (10, 30 and 100 mg/kg) following 12-15 days drug treatment on irritability following in utero valproic acid exposure-effect on repeated bottle brush reactivity in postnatal day 60-63 animals. Data represents mean±SEM number of aggressive behaviours (FIG. 13A), number of defensive behaviours (FIG. 13B) and irritability score (FIG. 13C) (sum of aggressive and defensive behaviours) over four daily sessions each of ten 10-second trials (n=8). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIGS. 14A-B show the effect of treatment with drug substance (10, 30 and 100 mg/kg) following 11 days drug treatment on social deficits following in utero valproic acid exposure-effect on social behavior in postnatal day 59 animals (FIG. 14A). Data represents mean±SEM total time spent engaged in active social behaviors (sec), and total time (sec) spent in social, center, and non-social chambers of the apparatus during a 5-minute test (n=8) (FIG. 14B). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIGS. 15A-D show the effect of drug substance (10, 30 and 100 mg/kg) following 8 and 9 days drug treatment on (A&B) circling behavior (FIG. 15A Day 1 and FIG. 15B Day 2) as measured by frequency of 360° circling and (C&D) grooming behavior (FIG. 15C Day 1 and FIG. 15D Day 2) as measured by duration of time spent grooming following in utero valproic acid exposure in the open field during two 10-minute trials over two days (on days 1 and 2) (n=8). Data represents mean±SEM. Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, * ** or ####p<0.0001.

FIGS. 16A-C show the effect of drug substance (10, 30 and 100 mg/kg) following 10 days drug treatment following in utero valproic acid exposure-effect on marble burying behavior in postnatal day 58 animals. Data represents mean±SEM number of buried marbles (covered by >50% substrate) (FIG. 16A), distance travelled (cm) (FIG. 16B), and duration of time spent digging (sec) in the open field during a 10-minute trial (n=8) (FIG. 16C). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIG. 17 shows the effect of drug substance (10, 30 and 100 mg/kg) following 8 and 9 days drug treatment on open field locomotor activity following in utero valproic acid exposure. Data represents mean±SEM of total distance travelled (cm) in the open field during two 10-minute trials over two days, day one spontaneous locomotor activity and day 2 hyperactivity following habituation (n=8). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, *** or ###p<0.001, **** or ####p<0.0001.

FIGS. 18A-D show the effect of drug substance (10, 30 and 100 mg/kg) following 8 and 9 days drug treatment on exploratory behavior following in utero valproic acid exposure. Data represents mean #SEM frequency of center zone crossings on (FIG. 18A) day one and (FIG. 18B) day two, and duration spent in the center zone (sec) on (FIG. 18C) day one and (FIG. 18D) day two of the open field during two 10-minute trials over two days (n=8). Statistical difference indicated by an asterisk * for VPA/Veh vs Veh/Veh comparison and by a hash # for comparisons between DS or risperidone treatment groups vs VPA/Veh groups; * or #p<0.05, ** or ##p<0.01, or ###p<0.001, **** or ####p<0.0001.

FIGS. 19A-B show the experimental design (FIG. 19A) and valproic acid-induced mouse model generation scheme (FIG. 19B) as described in Example 6.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About or Approximately: The terms “about” or “approximately”, as applied to one or more values of interest, refers to a value that is similar to a stated reference value. In certain embodiments, the term “about” or “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context. In certain embodiments, “about” refers to range of values that falls within 10% in either direction (greater than or less than, inclusive of endpoints) of the stated reference value

Administration: As used herein, the term “administration” typically refers to the administration of a composition to a patient. Those of ordinary skill in the art will be aware of a variety of routes that may, in appropriate circumstances, be utilized for administration to a patient, for example a human. For example, in embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In embodiments, administration may involve dosing that is intermittent (e.g., a plurality of doses separated in time) and/or periodic (e.g., individual doses separated by a common period of time) dosing. In embodiments, administration may involve continuous dosing (e.g., perfusion) for at least a selected period of time.

Adult: As used herein, refers to any organism which is fully grown or developed. Adult organisms include adult animals (e.g., mammals such as mice, rats, rabbits, non-human primates, and/or humans). In embodiments, an adult is a human. Adult humans are aged from about 18 and older. In embodiments, Adult humans are aged from about 18 to about 65 years.

Active Agent: In general, the term “active agent”, as used herein, may be used to refer to a compound or mixture of compounds that has a therapeutic agent. In embodiments, an agent may be utilized in isolated or pure form; In embodiments, an agent may be utilized in crude form.

Agitation: as used herein, refers to behaviors consistent with emotional distress (e.g., rapid changes in mood, irritability, outbursts) which manifest as excessive motor activity, verbal aggression, or physical aggression. Agitation commonly occurs in Alzheimer's and dementia.

Amelioration: as used herein, refers to the prevention, reduction or palliation of a state, or improvement of the state of a patient. Amelioration includes but does not require complete recovery or complete prevention of a disease, disorder or condition (e.g., radiation injury).

Biologically active: as used herein, refers to an observable biological effect or result achieved by an agent or entity of interest. For example, in embodiments, a specific binding interaction is a biological activity. In embodiments, modulation (e.g., induction, enhancement, or inhibition) of a biological pathway or event is a biological activity. In embodiments, presence or extent of a biological activity is assessed through detection of a direct or indirect product produced by a biological pathway or event of interest.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gel, liquid, or solid.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In embodiments, determining involves manipulation of a physical sample. In embodiments, determining involves consideration and/or manipulation of data or information, for example utilizing a computer or other processing unit adapted to perform a relevant analysis. In embodiments, determining involves receiving relevant information and/or materials from a source. In embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic agent) for administration to a patient. Typically, each such unit contains a predetermined quantity of active agent. In embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular patient is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a patient, typically separated by periods of time. In embodiments, a given therapeutic agent has a recommended dosing regimen, which may involve one or more doses. In embodiments, a dosing regimen comprises a plurality of doses each of which is separated in time from other doses. In embodiments, individual doses are separated from one another by a time period of the same length; In embodiments, a dosing regimen comprises a plurality of doses and at least two different time periods separating individual doses. In embodiments, all doses within a dosing regimen are of the same unit dose amount. In embodiments, different doses within a dosing regimen are of different amounts. In embodiments, a dosing regimen comprises a first dose in a first dose amount, followed by one or more additional doses in a second dose amount different from the first dose amount. In embodiments, a dosing regimen comprises a first dose in a first dose amount, followed by one or more additional doses in a second dose amount same as the first dose amount. In embodiments, a dosing regimen is correlated with a desired or beneficial outcome when administered across a relevant population (i.e., is a therapeutic dosing regimen).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Improve, increase, inhibit or reduce: As used herein, these terms, or grammatically comparable comparative terms, indicate values that are relative to a comparable reference measurement. For example, in embodiments, an assessed value achieved with an agent of interest may be “improved” relative to that obtained with a comparable reference agent. Alternatively or additionally, in embodiments, an assessed value achieved in a patient of interest may be “improved” relative to that obtained in the same patient under different conditions (e.g., prior to or after an event such as administration of an agent of interest), or in a different, comparable subject (e.g., in a comparable subject or system that differs from the patient or system of interest in presence of one or more indicators of a particular disease, disorder or condition of interest, or in prior exposure to a condition or agent, etc.). In embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Irritability: as used herein refers to a transdiagnostic mood state. The concept is sometimes related to other neurobehavioral constructs, including aggression, violent outbursts, hostility, bad temper, anger, poor impulse control, tension, uncooperativeness, excitement, and hyperarousal. Terminology varies across indications. ‘Irritability’ is commonly used in ASD to describe a wider range of behaviors that also include aggression, self-injurious behavior, tantrums/outbursts, and quickly changing moods.

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. In embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Isomer: As is known in the art, many chemical entities (in particular many organic molecules and/or many small molecules) can exist in a variety of structural (e.g., geometric/conformational) and/or optical isomeric forms. For example, any chiral center can exist in R and S configurations; double bonds can exist in Z and E conformational isomers, certain structural elements can adopt two or more tautomeric forms, etc. In embodiments, as will be clear to those skilled in the art from context, depiction of or reference to a particular compound structure herein may represent all structural and/or optical isomers thereof. In embodiments, as will be clear to those skilled in the art from context, depiction of or reference to a particular compound structure herein is intended to encompass only the depicted or referenced isomeric form. In embodiments, compositions including a chemical entity that can exist in a variety of isomeric forms include a plurality of such forms; In embodiments such compositions include only a single form. For example, In embodiments, compositions including a chemical entity that can exist as a variety of optical isomers (e.g., stereoisomers, diastereomers, etc.) include a racemic population of such optical isomers; In embodiments such compositions include only a single optical isomer and/or include a plurality of optical isomers that together retain optical activity. Where there exists two or more isomers within a composition they may exist as a mixture with various ratios.

Mixture: The phrase “mixture” describes a combination of two or more different compounds or agents which occur within the same composition.

Moiety: Those skilled in the art will appreciate that a “moiety” is a defined chemical group or entity with a particular structure and/or or activity, as described herein.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In embodiments, a patient is a human. In embodiments, a patient is suffering from or susceptible to one or more disorders or conditions. In embodiments, a patient displays one or more symptoms of a disorder or condition. In embodiments, a patient has been diagnosed with one or more disorders or conditions. In embodiments, the patient is receiving or has received certain therapy to diagnose and/or to treat a disease, disorder, or condition.

Pharmaceutical composition: As used herein, the term “pharmaceutical composition” refers to composition comprising an active agent, formulated together with one or more pharmaceutically acceptable carriers. In embodiments, the active agent is present in an amount in the pharmaceutical composition appropriate for administration in a therapeutic regimen that shows a statistically significant probability of achieving a predetermined therapeutic effect when administered to a relevant population. In embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Predetermined: By predetermined is meant deliberately selected, for example as opposed to randomly occurring or achieved.

Prevent or prevention: As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Predominantly present: The term “predominantly present”, as used herein, refers to the quantity of an entity (e.g., a specific cannabinoid or isomer thereof) in a preparation or composition. For example, a cannabinoid may be predominantly present if it is at least about 50%, about 55%, about 60%, about 65%, about 70%, about 75%, about 80%, about 85%, about 90%, about 95%, about 96%, about 97%, about 98%, about 99%, or about 100% of the total cannabinoid in the preparation or composition.

Prevention: The term “prevention”, as used herein, refers to a delay of onset, and/or reduction in frequency and/or severity of one or more symptoms of a particular disease, disorder or condition. In embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. Prevention may be considered complete when onset of a disease, disorder or condition has been delayed for a predefined period of time.

Pure: As used herein, an agent or entity is “pure” if it is substantially free of other components. For example, a preparation that contains more than about 90% of a particular agent or entity is typically considered to be a pure preparation. In embodiments, an agent or entity is at least 91%, at least 92%, at least 93%, at least 94%, at least 95%, at least 96%, at least 97%, at least 98%, or at least 99% pure.

Reference: As used herein describes a standard or control relative to which a comparison is performed. For example, in embodiments, an agent, animal, individual, population, sample, sequence or value of interest is compared with a reference or control agent, animal, individual, population, sample, sequence or value. In embodiments, a reference composition may comprise one or more synthetic cannabinoids. In embodiments a reference composition may contain different types of cannabinoids, different isomeric forms of cannabinoids, different distribution of cannabinoids, different quantities of cannabinoids, etc. as compared to a test composition. In embodiments, a reference or control is tested and/or determined substantially simultaneously with the testing or determination of interest. In embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. Response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. The exact response criteria can be selected in any appropriate manner, provided that when comparing groups of cells or subjects, the groups to be compared are assessed based on the same or comparable criteria for determining response rate. One of ordinary skill in the art will be able to select appropriate criteria.

Solid form: As is known in the art, many chemical entities (in particular many organic molecules and/or many small molecules) can adopt a variety of different solid forms such as, for example, amorphous forms and/or crystalline forms (e.g., polymorphs, hydrates, solvates, etc.). In embodiments, such entities may be utilized as a single such form (e.g., as a pure preparation of a single polymorph). In embodiments, such entities may be utilized as a mixture of such forms.

Subject: As used herein, the term “subject” refers an organism, typically a mammal (e.g., a human, in embodiments including prenatal human forms). In embodiments, a subject refers to any organism (e.g., mammals such as mice, rats, rabbits, non-human primates, and humans; insects; worms; etc.) to which a provided compound or composition is administered in accordance with the present disclosure e.g., for experimental, diagnostic, prophylactic, and/or therapeutic purposes. In embodiments, a subject is suffering from a relevant disease, disorder or condition. In embodiments, a subject is susceptible to a disease, disorder, or condition. In embodiments, a subject displays one or more symptoms or characteristics of a disease, disorder or condition. In embodiments, a subject does not display any symptom or characteristic of a disease, disorder, or condition. In embodiments, a subject is someone with one or more features characteristic of susceptibility to or risk of a disease, disorder, or condition. In embodiments, a subject is a patient. In embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In embodiments, an individual who is susceptible to a disease, disorder and/or condition may not have been diagnosed with the disease, disorder, and/or condition. In embodiments, an individual who is susceptible to a disease, disorder, and/or condition may exhibit symptoms of the disease, disorder, and/or condition. In embodiments, an individual who is susceptible to a disease, disorder, and/or condition may not exhibit symptoms of the disease, disorder, and/or condition. In embodiments, an individual who is susceptible to a disease, disorder, and/or condition will develop the disease, disorder, and/or condition. In embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” in general refers to any active agent that elicits a desired pharmacological effect when administered to an organism. A therapeutic agent is distinct from In embodiments, an agent is considered to be a therapeutic agent if it demonstrates a statistically significant effect across an appropriate population. In embodiments, the appropriate population may be a population of model organisms. In embodiments, an appropriate population may be defined by various criteria, such as a certain age group, gender, genetic background, preexisting clinical conditions, etc. In embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. Therapeutic agent may be used interchangeably with drug substance herein.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, active ingredient, preparation, composition, and/or formulation) that elicits a desired effect (e.g., a desired biological, clinical, or pharmacological effect or response) when administered as part of a therapeutic regimen. In embodiments, a therapeutically effective amount of a substance is an amount that is sufficient, when administered to a subject suffering from or susceptible to a disease, disorder, and/or condition, to treat, diagnose, prevent, reduce the severity of, stabilize one or more characteristics of, and/or delay the onset of the disease, disorder, and/or condition. In embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In embodiments, a therapeutically effective amount is administered in a single dose; In embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. In embodiments, treatment refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. Treatment may be administered to a subject who does not exhibit signs of a disease, disorder, and/or condition. In embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In embodiments, a unit dose contains an entire single dose of the agent. In embodiments, more than one unit dose is administered to achieve a total single dose. In embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### DETAILED DESCRIPTION

Cannabinoids are natural and synthetic compounds structurally or pharmacologically related to the constituents of the cannabis plant or to the endogenous agonists (endocannabinoids) of the cannabinoid receptors CB1 or CB2. The only way in nature in which these compounds are produced is by the cannabis plant. Cannabis is a genus of flowering plants in the family Cannabaceae, comprising the species Cannabis sativa, Cannabis indica, and Cannabis ruderalis (sometimes considered as part of Cannabis sativa).

Cannabis plants comprise a highly complex mixture of compounds. At least 568 unique molecules have been identified. Among these compounds are cannabinoids, terpenoids, terpenes, carotenoids, sugars, fatty acids, sterols, flavonoids, other hydrocarbons, nitrogenous compounds, and amino acids. It is known that non-cannabinoid components may also have therapeutic potential. The most notable being flavonoids and terpenes for uses including but not limited to cancer, inflammation, excessive reactive oxygen species in cells, and enhancement of neuroprotection.

Cannabinoids exert their physiological effects through a variety of receptors including, but not limited to, adrenergic receptors, cannabinoid receptors (CB1 and CB2), GPR55, GPR3, or GPR5. The principal cannabinoids present in cannabis plants are cannabinoid acids tetrahydrocannabinolic acid (THCA) and cannabidiolic acid (CBDA) with small amounts of their respective neutral (decarboxylated) cannabinoids. In addition, cannabis may contain lower levels of other minor cannabinoids. Chemical composition, pharmacological profiling, and complete physiological effects of these medicinal plants, and more importantly the extracts from cannabis, remain to be fully understood.

Crude extracts from cannabis plants containing CBD have been used by patients suffering from diseases and disorders. However, such crude products are unsuitable for use in pharmaceutical formulations. Those seeking to prepare more consistent CBD preparations for use in treating diseases or disorders have made a concerted effort to either prepare CBD synthetically or attempt to remove all compounds other than CBD, particularly psychoactive compounds such as THC, from plant derived cannabinoids.

The present invention encompasses the surprising discovery that a complex botanical mixture, comprising one or more additional cannabinoid and non-cannabinoid components in their specific amounts, and suitable for pharmaceutical use, exhibits enhanced therapeutic efficacy.

### Cannabinoids

Cannabinoids are a class of compounds which may be derived naturally from the cannabis plant or produced synthetically via chemical synthesis.

More than 100 different cannabinoids produced by cannabis have been identified. These cannabinoids can be split into different groups as follows: phytocannabinoids; endocannabinoids and synthetic cannabinoids (which may be novel cannabinoids or synthetically produced versions of phytocannabinoids or endocannabinoids).

Phytocannabinoids are cannabinoids that originate from nature and can be found in the cannabis plant. Phytocannabinoids can be isolated from plants to produce a highly purified extract. Phytocannabinoids may be obtained as either the neutral (decarboxylated form) or the carboxylic acid form depending on the method used to extract the cannabinoids from plant material. For example, it is known that heating the carboxylic acid form will cause most of the carboxylic acid form to decarboxylate into the neutral form. Phytocannabinoids can only be produced from plants, however versions of phytocannabinoids may be produced synthetically via chemical synthesis.

Endocannabinoids are endogenous lipid-based retrograde neurotransmitters that bind to cannabinoid receptors, and cannabinoid receptor proteins that are expressed throughout the mammalian central nervous system (including the brain) and peripheral nervous system. The endocannabinoid system is involved in regulating a variety of physiological and cognitive processes including fertility, pregnancy, during pre- and postnatal development, appetite, pain-sensation, mood, and memory, and in mediating the pharmacological effects of cannabis.

“Synthetic cannabinoids” are compounds that have a cannabinoid-like structure and are manufactured using chemical means rather than by the plant.

Certain cannabinoids are described in more detail below.

### Cannabidiol (CBD)

Cannabidiol (CBD) is a cannabinoid designated chemically as 2-[(1R,6R)-3-Methyl-6-(1-methylethenyl)-2-cyclohexen-1-yl]-5-pentyl-1,3-benzenediol. Its empirical formula is C21H3002 and its molecular weight is 314.46. CBD is a cannabinoid that naturally occurs in the Cannabis sativa L. plant. CBD is a white to pale yellow crystalline solid which is insoluble in water and soluble in organic solvents.

CBD is a major cannabinoid constituent of Cannabis species, such as the hemp plant (Cannabis sativa). Unlike other cannabinoids, such as THC, CBD does not bind CB1 or CB2, or its binding to the receptors is negligible in terms of inducing a pharmacological effect. Thus, CBD does not cause the central or peripheral nervous system effects mediated by the CB1 or CB2 receptors. CBD has little or no psychotropic (cannabimimetic) activity and its molecular structure and properties are substantially different from those of other cannabinoids.

CBD administration has been the subject of research in an attempt to provide an alternative treatment for various diseases and disorders which may respond to such treatment.

In embodiments, CBD is isolated from a cannabis plant. In embodiments, CBD is prepared synthetically. In embodiments, CBD is present as (−)-trans-CBD.

### Tetrahydrocannabinol (THC)

THC is the principal psychoactive constituent of cannabis.

The THC molecule may exist as four distinct chiral forms. THC has 2 stereocenters which in turn enable the existence of 4 stereoisomers (+)-trans-THC; (−)-trans-THC; (+)-cis-THC and (−)-cis-THC. THC commonly occurs in nature as the (−)-trans-THC isomer (Hollister, 1970)

The THC molecule mostly occurs as (−)-trans-Δ⁹-tetrahydrocannabinol, however the (−)-trans-Δ⁸-tetrahydrocannabinol homolog is also known to exist. In embodiments, the compound THC of the present disclosure refers to either the Δ⁸ or the Δ⁹ homolog.

In embodiments, THC is isolated from a cannabis plant. In embodiments, THC is prepared synthetically. In embodiments, THC is present as (−)-trans-THC. In embodiments, THC is present as (−)-cis-THC. In embodiments, THC is present as (+)-trans-THC. In embodiments, THC is present as (+)-cis-THC.

In embodiments the THC is present as a mixture of isomers. In embodiments the mixture will comprise one or more of (+)-trans-THC, (−)-trans-THC, (+)-cis-THC and (−)-cis-THC.

### Cannabidivarin (CBDV)

CBDV is a homolog of CBD, with the side-chain shortened by two methylene bridges. In embodiments CBDV is isolated from a cannabis plant. In embodiments CBDV is prepared synthetically. In embodiments, CBDV is present as (−)-trans-CBDV.

### Cannabidiol-C1 (CBD-C1)

In embodiments CBD-C1 is isolated from a cannabis plant. In embodiments CBD-C1 is prepared synthetically. In embodiments, CBD-C1 is present as (−)-trans-CBD-C1.

### Cannabidiol-C4 (CBD-C4)

In embodiments CBD-C4 is isolated from a cannabis plant. In embodiments CBD-C4 is prepared synthetically. In embodiments, CBD-C4 is present as (−)-trans-CBD-C4.

### Cannabigerol (CBG)

In embodiments CBG is isolated from a cannabis plant. In embodiments CBG is prepared synthetically.

### Hydroxy-Cannabidiol (OH-CBD)

In embodiments OH-CBD is isolated from a cannabis plant. In embodiments OH-CBD is prepared synthetically.

### Cannabichromene (CBC)

In embodiments CBC is isolated from a cannabis plant. In embodiments CBC is prepared synthetically.

### Cannabielsoin I (CBE I)

In embodiments CBE I is isolated from a cannabis plant. In embodiments CBE I is prepared synthetically.

### Cannabinol (CBN)

In embodiments CBN is isolated from a cannabis plant. In embodiments CBN is prepared synthetically.

### Non-Cannabinoid Components of the Cannabis Plant

### Terpenes

Terpenes are abundant in cannabis and are found in the trichome of the cannabis plant. Terpenes have a wide range of therapeutic uses such as providing pain relief, neuroprotective, antibacterial, anti-inflammatory, anti-microbial, and antioxidant properties. Terpenes have been used at large scales in modern medication for various treatment drugs.

There are thousands of terpenes in existence with over 150 identified in cannabis. Each cannabis strain has a unique terpene profile, depending on how the strain was grown. The terpene profile of strains can also vary between growers.

Caryophyllene is a type of terpene that can take the form of Beta-Caryophyllene, Caryophyllene Oxide and Trans Caryophyllene. Beta-Caryophyllene is the most common caryophyllene in cannabis and binds directly with the CB2 receptor. Caryophyllene oxide is a terpene that results from the oxidation of beta-caryophyllene. Trans-Caryophyllene is a sesquiterpene that has similar medicinal properties to other terpenes but does not activate the endocannabinoid system (unlike Beta-Caryophyllene).

Alpha-bisabolol is another sesquiterpene found in cannabis. The use of α-bisabolol as an anti-inflammatory agent is ubiquitous. This compound also exhibits several other pharmacological properties such as analgesic, antibiotic and anticancer activities.

In embodiments, terpene is present as trans-caryophyllene and/or alpha-bisabolol. In embodiments, terpene is present at about 1.0 to about 3.0% w/w of the composition.

### Sterols

Sterols are found in the stem bark and root of cannabis plants. Plant sterols are used to reduce plasma cholesterol levels and may be used for diseases such as heart disease and obesity. Other therapeutic uses are poorly understood.

In embodiments, sterol is present at about 0.5 to about 2.0% w/w of the composition.

### Methods of Making Cannabinoid Compositions

In the context of this application a “botanical drug substance” is an extract derived from cannabis plant material, which extract fulfils the definition of “botanical drug substance” provided in the Guidance for Industry Botanical Drug Products Draft Guidance, August 2000, US Department of Health and Human Services, Food and Drug Administration Center for Drug Evaluation and Research of: “A drug substance derived from one or more plants, algae, or macroscopic fungi. It is prepared from botanical raw materials by one or more of the following processes: pulverization, decoction, expression, aqueous extraction, ethanolic extraction, or other similar processes.”

“Plant material” is defined as a plant or plant material (e.g., bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Center for Drug Evaluation and Research.

The method of the invention may be used to extract cannabinoids from a specified and defined plant material known to contain such cannabinoids. Most typically, but not necessarily, the “plant material” will be “plant material” or “botanical raw material” derived from one or more cannabis plants. Most typically, but not necessarily, the one or more cannabis plants will be a specified and defined cannabis plant bred to produce a high yield of CBD.

The term “Cannabis plant(s)” encompasses wild type Cannabis sativa and also variants thereof, including cannabis chemovars which naturally contain different amounts of the individual cannabinoids, Cannabis sativa plants which are the result of genetic crosses, self-crosses or hybrids thereof. The term “Cannabis plant material” is to be interpreted accordingly as encompassing plant material derived from one or more cannabis plants. For the avoidance of doubt, it is hereby stated that “cannabis plant material” includes dried cannabis biomass. In embodiments, at least a portion of the cannabinoid acids in such cannabis plant material are decarboxylated.

### Cannabis Plants

The present invention utilizes cannabis plants and varieties bred to have specified, predetermined cannabinoid profiles and content. In embodiments, a cannabinoid is CBD, THC, CBDA, CBDV, CBD-C1, CBD-C4, or a combination thereof. In embodiments, cannabis plants have specified, predetermined terpene profiles and content. In embodiments, cannabis plants have specified, predetermined sesquiterpene profiles and content. In embodiments, a cannabis plant is a Cannabis sativa, Cannabis indica, or Cannabis ruderalis plant.

### Cannabis Cultivation

In embodiments, cannabis plants are propagated from cuttings taken from a mother plant. In embodiments, a mother plant originates from a single seed source. In embodiments, a crop is produced through asexual propagation. In embodiments, all of the plants in a crop are all female. In embodiments, propagation using cuttings controls genotype consistency.

In embodiments, the growing cycle is about 12 weeks. In embodiments, through controlled growing conditions cannabis plants take about 12 weeks to reach maturity. In embodiments, cannabis plants are irrigated throughout their growing cycle with potable quality water. In embodiments, no synthetic herbicides or pesticides are used in the cultivation of cannabis plants. In embodiments, stringent hygiene conditions may be utilized to reduce ingress of pests and diseases, particularly in the absence of herbicides or pesticides. In embodiments, control of growing conditions to reduce or eliminate environmental stresses is utilized to optimize plant material yield, cannabinoid content, and/or control disease. In embodiments, environmental stresses may include drought, insufficient light, improper timing of light cycle, and unfavourable temperatures. In addition, regular inspection of the plants during the growing cycle allows for the detection of any rogue plants and pests. Rogue male plants may arise, though weeds should be absent due to the controlled growing conditions and media. Frequent inspections and biological control methods are used to manage any pests and diseases that may occur.

In embodiments, through strict control of growing conditions the Cannabis plants reach maturity in approximately 12 weeks. In embodiments, in the last weeks of growth, dense resinous flowers develop. In embodiments, by the end of approximately week 11 the cannabinoid biosynthesis has slowed markedly, and the plants are ready for harvest.

### Cannabis Harvest and Processing

In embodiments, the entire plant is cut and dried in a temperature and/or humidity controlled environment. In embodiments, the temperature is about 21° C. In embodiments, the humidity is about 38-45% RH.

THC and CBD are present as the carboxylic acids THCA and CBDA in the BRM. The acid forms slowly decarboxylate over time during drying. The leaves and flowers are stripped from the larger stems to provide the Botanical Raw Material (BRM). Under conditions of storage the loss on drying reaches equilibrium of approximately 10%. The storage conditions for the dried BRM will be dependent on the physical status of the BRM. In embodiments, BRM is stored protected from light. In embodiments, BRM is stored at about 15-25° C. In embodiments, BRM is stored at about −20° C. In embodiments, BRM is stored at about 20° C. In embodiments, BRM is stored at about 38-42% RH.

Summary of exemplary production of a BRM:
Harvest plants
Drying (in absence of light)
Production of Botanical Raw Material (BRM) which comprises cannabinoid acids
Milling to less than 2000 μm to reduce particle size
Decarboxylation of cannabinoid acids to their neutral form (e.g., CBDA to CBD)

### Decarboxylation

THC and CBD present as their respective carboxylic acids in Cannabis plants. In order to extract THC or CBD from cannabis plant material, it is necessary to convert the storage precursor compounds of THCA and CBDA into their more readily extractable and pharmacologically active forms. THC and CBD acids slowly decarboxylate naturally over time. The traditional way to increase rate of decarboxylation is by the application of heat. However, THCA is converted not only to THC, but also to another cannabinoid, cannabinol (CBN).

The decarboxylation procedure is generally carried out within the preparation of the starting material or botanical raw material (BRM), prior to the initiation of the extraction process.

### Overview of Exemplary Extraction Process:

The BDS may be extracted from decarboxylated BRM using liquid carbon dioxide methodology. This involves continuously passing liquefied carbon dioxide through the chopped biomass, which is contained in a high-pressure vessel. The crude extract is dissolved in ethanol, cooled to a low temperature then filtered to remove precipitated constituents such as waxes. Removing ethanol and water in vacuo produces BDS containing either high concentrations of CBD or THC, depending on the biomass used.

Additional methods regarding the purification and characterization of CBD preparations are disclosed and described in EP 2,311,475, the content of which is hereby incorporated by reference in its entirety.

### Preparation of Drug Substance

The following describes the production of the presently claimed complex botanical mixture which has a known and constant composition which was used in the Examples below.

A reliable and consistent method was developed to achieve the desired therapeutic profile by blending two drug substance intermediates prepared from the same botanical raw material (BRM).

Both drug substance intermediates were obtained from the same milled BRM source, generated from the harvested and dried leaves and flowers of cultivated plants of Cannabis sativa L.

The BRM was thermally decarboxylated to provide the neutral form of the cannabinoids. Decarboxylated BRM then underwent extraction using liquid CO₂, to yield a crude extract comprising cannabinoids and non-cannabinoid components in specific amounts (Drug Substance Intermediate 1, specification provided in Table 2).

A batch of the crude extract underwent further purification by crystallization to produce Drug Substance Intermediate 2 (specification provided in Table 3). The crystallization process specifically removed other cannabinoids and plant components to yield greater than 95% (w/w) CBD.

Intermediate 2 was then blended back in with Intermediate 1 material to achieve the desired weight ratio, of 70% Intermediate 1 to 30% Intermediate 2.

The resultant complex botanical mixture was then winterized to remove plant waxes, to yield the drug substance, which was held as a solution in acetone in a 1:1 solution of winterized Intermediate 1/Intermediate 2 mixture to acetone, the acetone being present for stability and ease of handling. The acetone, an inactive, was subsequently removed during manufacture of the final drug product.

In summary, drug substance can be produced as follows:
a) Milled BRM
b) Decarboxylation
c) Extraction with CO₂=>Intermediate 1
d) Crystallisation of Intermediate 1=>Intermediate 2
e) Mixing of 70% Intermediate 1 and 30% Intermediate 2
f) Winterization of combined Intermediate 1/Intermediate 2 mixture=>Drug substance in acetone.

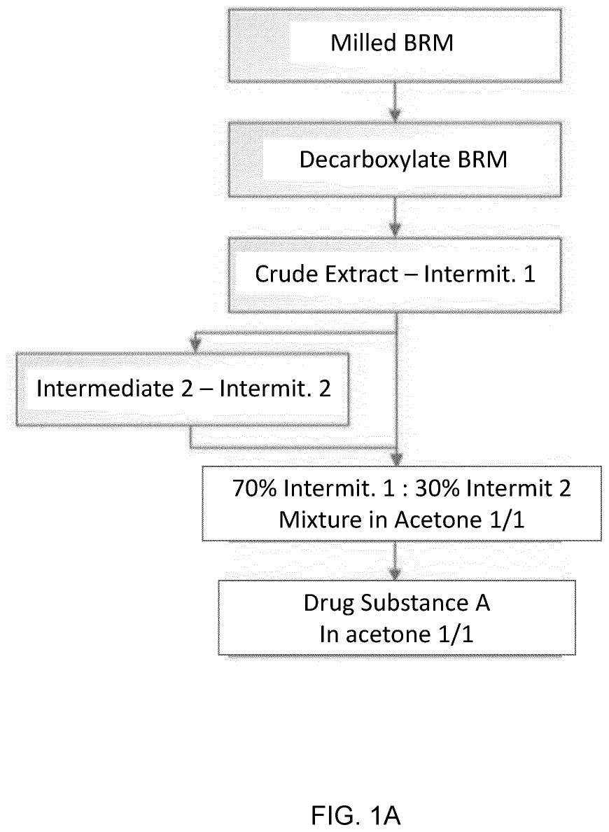

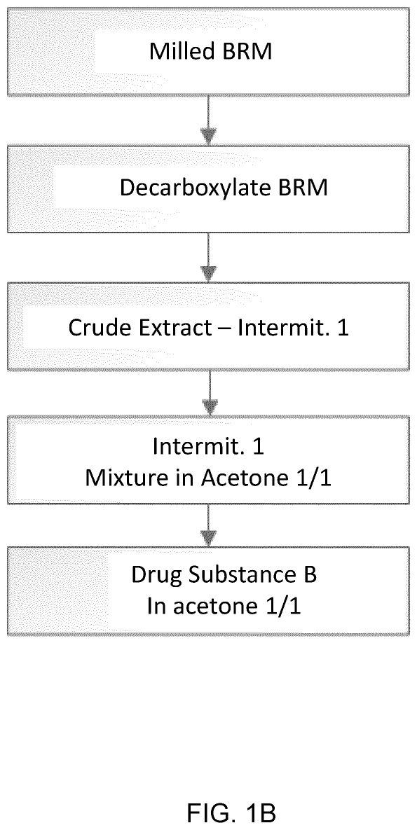

In embodiments, the drug substance is produced by following the manufacturing process as shown in FIG. 1A. In embodiments, the following extraction conditions are used:
a) Pressure: 85/95 bar (+/−10 bar tolerance)
b) Temperature: 25° C.±4° C.
c) CO₂ charge: 17-20 kg of CO₂/kg of BRM.

The botanical starting material, the two drug substance intermediates and the botanical drug substance (BDS) may be controlled by specifications in order to produce a consistent profile of cannabinoid and non-cannabinoid components in the BDS. An exemplary botanical starting material specification for decarboxylated cannabis plant material is described in Table 1 below. In embodiments, the isomeric content for each cannabinoid may also be specified.

TABLE 1
Exemplary Botanical Starting Material Specification

| | % w/w | | | | | | |
|---|---|---|---|---|---|---|---|
| Decarboxylated | | | Alpha- | Trans- | | | |
| BRM | CBD | Cis-THC | Bisabolol | Phytol | Terpenes | THC | CBG |
| MEAN | 13.68 | 0.09 | 0.077 | 0.045 | 1.166 | 0.70 | 0.72 |
| MIN | 10.77 | 0.04 | 0.0003 | 0.028 | 0.664 | 0.40 | 0.34 |
| MAX | 15.92 | 0.13 | 0.120 | 0.064 | 1.777 | 0.87 | 1.06 |
| SD | 1.20 | 0.02 | 0.026 | 0.009 | 0.0248 | 0.10 | 0.14 |
| Approximate ratio | | 195.43 | 177.66 | 304 | 11.73 | 19.78 | 19.77 |
| of CBD to other | | | | | | | |
| cannabinoid | | | | | | | |

TABLE 2
Intermediate 1 Specification

| Cannabinoid | Concentration | |
|---|---|---|
| CBD | 60-75% | w/w |
| Trans-THC | 2-3% | w/w |
| Cis-THC | 0.5-1.5% | w/w |
| CBDA | NMT 0.2% | w/w |
| CBDV | 0.2-1.0% | w/w |
| CBD-C1 | NMT 0.2% | w/w |
| CBD-C4 | 0.1-0.5% | w/w |
| OH-CBD | 0.2-1.0% | w/w |
| CBE I | NMT 0.3% | w/w |
| CBE II | NMT 0.1% | w/w |
| CBG | 0.5-2.5% | w/w |
| CBN | NMT 0.2% | w/w |
| CBL | NMT 0.2% | w/w |
| CBC | 1-4% | w/w |
| THCV | NMT 0.1% | w/w |
| THC-C4 | NMT 0.1% | w/w |
| Total cannabinoids | 65-90% | w/w |

NMT: not more than

TABLE 3
Intermediate 2 Specification

| Cannabinoid | Concentration |
|---|---|
| CBD | NLT 95.0% w/w |

NLT: not less than

TABLE 4
Specification of Drug Substance in 50% w/w Acetone

| Component | Concentration | |
|---|---|---|
| CBD | 32.5-47.5% | w/w |
| Trans-THC | 0.2-1.5% | w/w |
| CBC | 0.5-2.0% | w/w |
| CBG | 0.1-2.0% | w/w |
| CBDV | 0.1-0.7% | w/w |
| CBD-C4 | 0.05-0.4% | w/w |
| Cis-THC | 0.05-0.8% | w/w |
| Total cannabinoids | 35-50% | w/w |
| Total terpenes | 0.5-1.5% | w/w |
| Total triglycerides | 0.4-1.5% | w/w |
| Total sterols | 0.25-1.0% | w/w |

TABLE 5
Specification of Drug Substance

| Component | Concentration | |
|---|---|---|
| CBD | 65-95% | w/w |
| Trans-THC | 0.4-3.0% | w/w |
| CBC | 1.0-4.0% | w/w |
| CBG | 0.2-4.0% | w/w |
| CBDV | 0.2-1.4% | w/w |
| CBD-C4 | 0.1-0.8% | w/w |
| Cis-THC | 0.1-1.6% | w/w |
| Total cannabinoids | 70-100% | w/w |
| Total terpenes | 1.0-3.0% | w/w |
| Total triglycerides | 0.8-3.0% | w/w |
| Total sterols | 0.5-2.0% | w/w |

In embodiments, the present disclosure provides a drug substance comprising a total cannabinoids concentration of about 50% w/w to about 100% w/w based on the weight of the drug substance (e.g., about 50% w/w, about 51% w/w, about 52% w/w, about 53% w/w, about 54% w/w, about 55% w/w, about 56% w/w, about 57% w/w, about 58% w/w, about 59% w/w, about 60% w/w, about 61% w/w, about 62% w/w, about 63% w/w, about 64% w/w, about 65% w/w, about 66% w/w, about 67% w/w, about 68% w/w, about 69% w/w, about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w, about 86% w/w, about 87% w/w, about 88% w/w, about 89% w/w, about 90% w/w, about 91% w/w, about 92% w/w, about 93% w/w, about 94% w/w, about 95% w/w, about 96% w/w, about 97% w/w, about 98% w/w, about 99% w/w, or about 100% w/w, including any values or ranges therebetween). In embodiments, the present disclosure provides a drug substance comprising total cannabinoids concentration of about 76% w/w to about 100% w/w. In embodiments, the drug substance comprises a total cannabinoids concentration of about 76% w/w to about 92% w/w. In embodiments, the drug substance comprises a total cannabinoids concentration of about 76% w/w to about 83% w/w. In embodiments, the drug substance comprises a total cannabinoids concentration of about 77% w/w to about 83% w/w. In embodiments, the drug substance comprises a total cannabinoids concentration of about 76.9% w/w to about 92.0% w/w.

In embodiments, the drug substance comprises CBD at a concentration of about 50% w/w to about 95% w/w, (e.g., about 50% w/w, about 51% w/w, about 52% w/w, about 53% w/w, about 54% w/w, about 55% w/w, about 56% w/w, about 57% w/w, about 58% w/w, about 59% w/w, about 60% w/w, about 61% w/w, about 62% w/w, about 63% w/w, about 64% w/w, about 65% w/w, about 66% w/w, about 67% w/w, about 68% w/w, about 69% w/w, about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w, about 86% w/w, about 87% w/w, about 88% w/w, about 89% w/w, about 90% w/w, about 91% w/w, about 92% w/w, about 93% w/w, about 94% w/w, or about 95% w/w, including any values or ranges therebetween) based on the total weight of the drug substance (e.g., botanical extract). In embodiments, the drug substance comprises about 50% w/w, about 55% w/w, about 60% w/w, about 65% w/w, about 70% w/w, about 75% w/w, about 80% w/w, about 85% w/w, about 90% w/w, or 95% w/w CBD based on the total weight of the therapeutic agent, including all value sand ranges therein. In embodiments, the drug substance comprises at least 60% w/w CBD based on the total weight of the drug substance. In embodiments, the drug substance comprises about 65% w/w to about 95% w/w CBD based on the total weight of the drug substance. In embodiments, the drug substance comprises about 69% w/w to about 84% w/w CBD based on the total weight of the drug substance. In embodiments, drug substance comprises about 77% w/w to about 84% w/w CBD based on the total weight of the drug substance. In embodiments, the drug substance comprises about 69% w/w to about 75% w/w CBD based on the total weight of the drug substance. In embodiments, the drug substance comprises about 75% w/w CBD based on the total weight of the drug substance. In embodiments, the drug substance comprises about 73% w/w CBD based on the total weight of the drug substance. In embodiments, the drug substance comprises about 69.5% w/w to about 83.6% w/w CBD based on the total weight of the drug substance.

In embodiments, the present disclosure provides a drug substance, wherein the drug substance comprises cannabidiol (CBD) in amount of about 65% w/w to about 95% w/w based on the total weight of the drug substance, and the remainder comprises trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC).

In embodiments, the drug substance comprises trans-THC at a concentration of about 0.1% w/w to about 5.0% w/w, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, or about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5.0% w/w, including any values or ranges therebetween, based on the total weight of the drug substance. In embodiments, the drug substance comprises trans-THC at a concentration of about 1.3% w/w to about 1.9% w/w based on the total weight of the drug substance. In embodiments, the drug substance comprises trans-THC at a concentration of about 0.4% w/w to about 3.0% w/w based on the total weight of the drug substance.

In embodiments, the drug substance comprises CBC at a concentration of about 0.1% w/w to about 5.0% w/w, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, or about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5.0% w/w, including any values or ranges therebetween, based on the total weight of the drug substance. In embodiments, the drug substance comprises CBC at a concentration of about 1.0% w/w to about 4.0% w/w based on the total weight of the drug substance. In embodiments, drug substance comprises CBC at a concentration of about 1.5% w/w to about 2.3% w/w based on the total weight of the drug substance. In embodiments, the drug substance comprises CBC at a concentration of about 1.5% w/w to about 1.8% w/w based on the total weight of the drug substance. In embodiments, the drug substance comprises CBC at a concentration of about 1.7% w/w to about 2.3% w/w based on the total weight of the drug substance.

In embodiments, the drug substance comprises CBG at a concentration of about 0.1% w/w to about 5.0% w/w, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5.0% w/w, including any values or ranges therebetween, based on the total weight of the drug substance. In embodiments, the drug substance comprises CBG at a concentration of about 2.0% w/w to about 4.0% w/w based on the total weight of the drug substance. In embodiments, the drug substance comprises CBG at a concentration of about 1.0% w/w to about 4.0% w/w based on the total weight of the drug substance. In embodiments, the drug substance comprises CBG at a concentration of about 1.5% w/w to about 2.3% w/w based on the total weight of the drug substance. In embodiments, the drug substance comprises CBG at a concentration of about 0.5% w/w to about 1.5% w/w based on the total weight of the drug substance.

In embodiments, the drug substance comprises CBDV at a concentration of about 0.2% w/w to about 1.4% w/w, for example, about 0.1% w/w to about 5.0% w/w, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/, about 3.7% w/w, about 3.8% w/w, about 3.9% w/w, or about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5.0% w/w, including any values or ranges therebetween, based on the total weight of the drug substance. In embodiments, the drug substance comprises CBDV at a concentration of about 0.4% w/w to about 0.7% w/w based on the total weight of the drug substance. In embodiments, the drug substance comprises CBDV at a concentration of about 0.4% w/w based on the total weight of the drug substance. In embodiments, the drug substance comprises CBDV at a concentration of about 0.2% w/w to about 1.4% (w/w) CBDV based on the total weight of the drug substance.

In embodiments, the drug substance comprises CBD-C4 at a concentration of about 0.1% w/w to about 5.0% w/w, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/, about 3.7% w/w, about 3.8% w/w, about 3.9% w/w, or about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5.0% w/w, including any values or ranges therebetween, based on the total weight of the drug substance. In embodiments, the drug substance comprises CBD-C4 at a concentration of about 0.3% w/w to about 0.4% w/w based on the total weight of the drug substance. In embodiments, the drug substance comprises CBD-C4 at a concentration of about 0.2% w/w to about 0.3% w/w based on the total weight of the drug substance. In embodiments, the drug substance comprises CBD-C4 at a concentration of about 0.1% w/w to about 0.8% w/w based on the total weight of the drug substance.

In embodiments, the drug substance comprises cis-THC at a concentration of about 0.1% w/w to about 5.0% w/w, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, or about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5.0% w/w, including any values or ranges therebetween, based on the total weight of the drug substance. In embodiments, the drug substance comprises cis-THC at a concentration of about 0.6% w/w to about 0.9% w/w based on the total weight of the drug substance. In embodiments, the drug substance comprises cis-THC at a concentration of about 0.1% w/w to about 1.6% w/w based on the total weight of the drug substance. In embodiments, the drug substance comprises cis-THC at a concentration of about 0.6% w/w to about 0.7% w/w based on the total weight of the drug substance.

In embodiments, the drug substance comprises a weight ratio of CBD to CBD-C4 of about 85:1 to about 470:1 (e.g., about 85:1, about 90:1, about 95:1, about 100:1, about 115:1, about 120:1, about 125:1, about 130:1, about 135:1, about 1401, about 145:1, about 150:1, about 155:1, about 160:1, about 165:1, about 170:1, about 175:1, about 180 1, bout 185:1, about 190:1, about 195:1, about 200:1, about 215:1, about 220:1, about 225:1, about 230:1, about 235:1, about 240:1, about 245:1, about 250:1, about 255:1, about 260:1, about 265:1, about 270, about 275:1, about 280:1, about 285:1, about 290:1, about 295:1, about 300:1, about 305:1, about 310:1, about 315:1, about 320:1, about 325:1, about 330:1, about 335:1, about 340:1, about 345:1, about 350:1, about 355:1, about 360:1, about 365:1, about 370:1, about 375:1, about 380:1, about 385:1, about 390:1, about 395:1, about 400:1, about 405:1, about 410:1, about 415:1, about 420:1, about 425:1, about 430:1, about 435:1, about 440:1, about 445:1, about 450:1, about 455:1, about 460:1, about 465:1, or about 470:1, including all values and ranges therein). In embodiments, the drug substance comprises a weight ratio of CBD to CBD-C4 of about 278:1 to about 258:1. In embodiments, the drug substance comprises a weight ratio of CBD to CBD-C4 of about 174:1 to about 251:1. In embodiments, the drug substance comprises a weight ratio of CBD to CBD-C4 of about 174:1 to about 258:1. In embodiments, the drug substance comprises a weight ratio of CBD to CBD-C4 of about 117.5:1 to 340:1. In embodiments, the drug substance comprises a weight ratio of CBD to CBD-C4 of about 376:5. In embodiments, the drug substance comprises a weight ratio of CBD to CBD-C4 of about 368:1. In embodiments, the drug substance comprises a weight ratio of CBD to CBD-C4 of about 278.8:1.

In embodiments, the drug substance comprises a weight ratio of CBD to CBDV of about 48:1 to about 470:1 (e.g., about 48:1, about 50:1, about 55:1, about 60:1, about 65:1, about 70:1, about 75:1, about 80:1, about 85:1, about 90:1, about 95:1, about 100:1, about 115:1, about 120:1, about 125:1, about 130:1, about 135:1, about 1401, about 145:1, about 150:1, about 155:1, about 160:1, about 165:1, about 170:1, about 175:1, about 180 1, bout 185:1, about 190:1, about 195:1, about 200:1, about 215:1, about 220:1, about 225:1, about 230:1, about 235:1, about 240:1, about 245:1, about 250:1, about 255:1, about 260:1, about 265:1, about 270, about 275:1, about 280:1, about 285:1, about 290:1, about 295:1, about 300:1, about 305:1, about 310:1, about 315:1, about 320:1, about 325:1, about 330:1, about 335:1, about 340:1, about 345:1, about 350:1, about 355:1, about 360:1, about 365:1, about 370:1, about 375:1, about 380:1, about 385:1, about 390:1, about 395:1, about 400:1, about 405:1, about 410:1, about 415:1, about 420:1, about 425:1, about 430:1, about 435:1, about 440:1, about 445:1, about 450:1, about 455:1, about 460:1, about 465:1, or about 470:1, including all values and ranges therein). In embodiments, the drug substance comprises a weight ratio of CBD to CBDV of about 99:1 to about 125:1. In embodiments, the drug substance comprises a weight ratio of CBD to CBDV of about 111:1 to about 209:1. In embodiments, the drug substance comprises a weight ratio of CBD to CBDV of about 67:1 to about 340:1. In embodiments, the drug substance comprises a weight ratio of CBD to CBDV of about 99:1 to 209:1. In embodiments, the drug substance comprises a weight ratio of CBD to CBDV of about 188:1. In embodiments, the drug substance comprises a weight ratio of CBD to CBDV of about 204:1. In embodiments, the drug substance comprises a weight ratio of CBD to CBDV of about 124.8:1.

In embodiments, the drug substance comprises a weight ratio of CBD to CBG of about 17:1 to about 470:1 (e.g., about 17:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 115:1, about 120:1, about 125:1, about 130:1, about 135:1, about 1401, about 145:1, about 150:1, about 155:1, about 160:1, about 165:1, about 170:1, about 175:1, about 180 1, bout 185:1, about 190:1, about 195:1, about 200:1, about 215:1, about 220:1, about 225:1, about 230:1, about 235:1, about 240:1, about 245:1, about 250:1, about 255:1, about 260:1, about 265:1, about 270, about 275:1, about 280:1, about 285:1, about 290:1, about 295:1, about 300:1, about 305:1, about 310:1, about 315:1, about 320:1, about 325:1, about 330:1, about 335:1, about 340:1, about 345:1, about 350:1, about 355:1, about 360:1, about 365:1, about 370:1, about 375:1, about 380:1, about 385:1, about 390:1, about 395:1, about 400:1, about 405:1, about 410:1, about 415:1, about 420:1, about 425:1, about 430:1, about 435:1, about 440:1, about 445:1, about 450:1, about 455:1, about 460:1, about 465:1, or about 470:1, including all values and ranges therein). In embodiments, the drug substance comprises a weight ratio of CBD to CBG of about 52:1 to about 93:1. In embodiments, the drug substance comprises a weight ratio of CBD to CBG of about 116:1 to about 150:1. In embodiments, the drug substance comprises a weight ratio of CBD to CBG of about 24:1 to about 340:1. In embodiments, the drug substance comprises a weight ratio of CBD to CBG of about 52:1 to 150:1. In embodiments, the drug substance comprises a weight ratio of CBD to CBG of about 77:1. In embodiments, the drug substance comprises a weight ratio of CBD to CBG of about 84:1. In embodiments, the drug substance comprises a weight ratio of CBD to CBG of about 99:1.

In embodiments, the drug substance comprises a weight ratio of CBD to cis-THC of about 43:1 to about 470:1 (e.g., about 43:1, about 45:1, about 50:1, about 55:1, about 60:1, about 65:1, about 70:1, about 75:1, about 80:1, about 85:1, about 90:1, about 95:1, about 100:1, about 115:1, about 120:1, about 125:1, about 130:1, about 135:1, about 1401, about 145:1, about 150:1, about 155:1, about 160:1, about 165:1, about 170:1, about 175:1, about 180 1, bout 185:1, about 190:1, about 195:1, about 200:1, about 215:1, about 220:1, about 225:1, about 230:1, about 235:1, about 240:1, about 245:1, about 250:1, about 255:1, about 260:1, about 265:1, about 270, about 275:1, about 280:1, about 285:1, about 290:1, about 295:1, about 300:1, about 305:1, about 310:1, about 315:1, about 320:1, about 325:1, about 330:1, about 335:1, about 340:1, about 345:1, about 350:1, about 355:1, about 360:1, about 365:1, about 370:1, about 375:1, about 380:1, about 385:1, about 390:1, about 395:1, about 400:1, about 405:1, about 410:1, about 415:1, about 420:1, about 425:1, about 430:1, about 435:1, about 440:1, about 445:1, about 450:1, about 455:1, about 460:1, about 465:1, or about 470:1, including all values and ranges therein). In embodiments, the drug substance comprises a weight ratio of CBD to cis-THC of about 86:1 to about 139:1. In embodiments, the drug substance comprises a weight ratio of CBD to cis-THC of about 99:1 to about 125:1. In embodiments, the drug substance comprises a weight ratio of CBD to cis-THC of about 86:1 to about 139:1. In embodiments, the drug substance comprises a weight ratio of CBD to cis-THC of about 59:1 to 340:1.

In embodiments, the drug substance comprises a weight ratio of CBD to trans-THC of about 23:1 to about 235:1 (e.g., about 23:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 115:1, about 120:1, about 125:1, about 130:1, about 135:1, about 1401, about 145:1, about 150:1, about 155:1, about 160:1, about 165:1, about 170:1, about 175:1, about 180 1, bout 185:1, about 190:1, about 195:1, about 200:1, about 215:1, about 220:1, about 225:1, about 230:1, or about 235:1, including all values and ranges therein). In embodiments, the drug substance comprises a weight ratio of CBD to trans-THC of about 40:1 to about 46:1. In embodiments, the drug substance comprises a weight ratio of CBD to trans-THC of about 46:1 to about 58:1. In embodiments, the drug substance comprises a weight ratio of CBD to trans-THC of about 31:1 to about 170:1. In embodiments, the drug substance comprises a weight ratio of CBD to trans-THC of about 41:1 to about 58:1. In embodiments, the drug substance comprises a weight ratio of CBD to trans-THC of about 47:1. In embodiments, the drug substance comprises a weight ratio of CBD to trans-THC of about 88:1.

In embodiments, the drug substance comprises a weight ratio of CBD to CBC of about 17:1 to about 94:1 (e.g., about 17:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, or about 94:1, including all values and ranges therein). In embodiments, the drug substance comprises a weight ratio of CBD to CBC of about 34:1 to about 49:1. In embodiments, the drug substance comprises a weight ratio of CBD to CBC of about 39:1 to about 50:1. In embodiments, the drug substance comprises a weight ratio of CBD to CBC of about 34:1 to about 50:1. In embodiments, the drug substance comprises a weight ratio of CBD to CBC of about 24:1 to about 68:1. In embodiments, the drug substance comprises a weight ratio of CBD to CBC of about 40:1. In embodiments, the drug substance comprises a weight ratio of CBD to CBC of about 42:1.

In embodiments, the drug substance of the present disclosure further comprises one or more of terpenes, triglycerides, sterols, fatty acids, and carotenoids.

In embodiments, the drug substance comprises terpenes ranging from about 1.0% w/w to about 8.0% w/w, for example, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5.0% w/w, about 5.1% w/w, about 5.2% w/w, about 5.3% w/w, about 5.4% w/w, about 5.5% w/w, about 5.6% w/w, about 5.7% w/w, about 5.8% w/w, about 5.9% w/w, about 6.0% w/w, about 6.1% w/w, about 6.2% w/w, about 6.3% w/w, about 6.4% w/w, about 6.5% w/w, about 6.6% w/w, about 6.7% w/w, about 6.8% w/w, about 6.9% w/w, about 7.0% w/w, about 7.1% w/w, about 7.2% w/w, about 7.3% w/w, about 7.4% w/w, about 7.5% w/w, about 7.6% w/w, about 7.7% w/w, about 7.8% w/w, about 7.9% w/w, or about 8.0% w/w, including any values or ranges therebetween, based on the total weight of the drug substance. In embodiments, the drug substance comprises terpenes at a concentration of about 2.0% w/w to about 7.3% w/w. In embodiments, the drug substance comprises terpenes at a concentration of about 1.0% w/w to about 3.0% w/w.

In embodiments, the drug substance comprises a weight ratio of CBD to terpenes of about 9:1 to about 94:1 (e.g., about 9:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, or about 94:1, including all values and ranges therein). In embodiments, the drug substance comprises a weight ratio of CBD to terpenes of about 32:1 to about 34:1. In embodiments, the drug substance comprises a weight ratio of CBD to terpenes of about 9:1 to about 20:1. In embodiments, the drug substance comprises a weight ratio of CBD to terpenes of about 9:1 to about 34:1. In embodiments, the drug substance comprises a weight ratio of CBD to terpenes of about 20:1 to about 34:1.

In embodiments, the drug substance comprises triglycerides at about 0.5% w/w to about 3.0% w/w, for example, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, or about 3.0% w/w, including any values or ranges therebetween, based on the total weight of the drug substance. In embodiments, the drug substance comprises triglycerides at a concentration of about 0.8% w/w to about 3.0% w/w. In embodiments, the drug substance comprises triglycerides at a concentration of about 1.6% w/w to about 2.6% w/w.

In embodiments, the drug substance comprises a weight ratio of CBD to triglycerides of about 23:1 to about 118:1 (e.g., about 23:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 115:1, or about 118:1, including all values and ranges therein). In embodiments, the drug substance comprises a weight ratio of CBD to triglycerides of about 26:1 to about 59:1. In embodiments, the drug substance comprises a weight ratio of CBD to triglycerides of about 23:1 to about 59:1. In embodiments, the drug substance comprises a weight ratio of CBD to triglycerides of about 59:1 to about 118:1. In embodiments, the drug substance comprises a weight ratio of CBD to triglycerides of about 23:1 to about 59:1.

In embodiments, the drug substance comprises sterols at about 0.5% w/w to about 3.0% w/w, for example, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, or about 3.0% w/w, including any values or ranges therebetween, based on the total weight of the drug substance. In embodiments, the drug substance comprises sterols at a concentration of about 0.5% w/w to about 2.0% w/w. In embodiments, the drug substance comprises sterols at a concentration of about 0.7% w/w to about 1.7% w/w.

In embodiments, the drug substance comprises a weight ratio of CBD to sterols of about 34:1 to about 188:1 (e.g., about 34:1, about 35:1, about 40:1, about 45:1, about 50:1, about 55:1, about 60:1, about 65:1, about 70:1, about 75:1, about 80:1, about 85:1, about 90:1, about 95:1, about 100:1, about 115:1, about 120:1, about 125:1, about 130:1, about 135:1, about 1401, about 145:1, about 150:1, about 155:1, about 160:1, about 165:1, about 170:1, about 175:1, about 180 1, bout 185:1, or about 188:1, including all values and ranges therein). In embodiments, the drug substance comprises a weight ratio of CBD to sterols of about 40:1 to about 94:1. In embodiments, the drug substance comprises a weight ratio of CBD to sterols of about 45:1 to about 134:1. In embodiments, the drug substance comprises a weight ratio of CBD to sterols of about 45:1 to about 94:1. In embodiments, the drug substance comprises a weight ratio of CBD to sterols of about 40:1 to about 134:1.

In embodiments, the drug substance of the present disclosure comprises one or more cannabinoids selected from the group consisting of about 0.1% w/w to about 5.0% w/w of trans-THC, about 0.1% w/w to about 5.0% w/w of CBC, about 0.1% w/w to about 5.0% w/w of CBG, about 0.1% w/w to about 5.0% w/w of CBDV, about 0.1% w/w to about 5.0% w/w of CBD-C4, or about 0.1% w/w to about 5.0% w/w of cis-THC.

In embodiments, the drug substance of the present disclosure comprises two or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC). In embodiments, the drug substance of the present disclosure comprises two or more cannabinoids selected from the group consisting of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

In embodiments, the drug substance of the present disclosure comprises three or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC). In embodiments, the drug substance of the present disclosure comprises three or more cannabinoids selected from the group consisting of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

In embodiments, the drug substance of the present disclosure comprises four or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC). In embodiments, the drug substance of the present disclosure comprises four or more cannabinoids selected from the group consisting of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

In embodiments, the drug substance of the present disclosure comprises five or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC). In embodiments, the drug substance of the present disclosure comprises five or more cannabinoids selected from the group consisting of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

In embodiments, the drug substance of the present disclosure comprises trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC). In embodiments, the drug substance of the present disclosure comprises about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), and about 0.1% w/w to about 5.0% w/w of cis-tetrahydrocannabinol (cis-THC).

### Methods of Treatment

In embodiments, the present disclosure provides methods of treating a disorder in a patient in need thereof by administering a drug substance of the present disclosure. In embodiments, the present disclosure provides a method of treating a neurological or neurodevelopmental disorder in a patient in need thereof, comprising administering a drug substance of the present disclosure. In embodiments, the present disclosure provides a method of treating a neurological or neurodevelopmental disorder in a patient in need thereof, comprising administering a pharmaceutical composition of the present disclosure to the patient. In embodiments, the neurological disorder or neurodevelopmental disorder is autism spectrum disorder (ASD), Alzheimer's Disease (AD), sleep disturbance, or psychiatric disorder. In embodiments, the psychiatric disorder is psychosis, depression, or schizophrenia. In embodiments, the neurological disorder or neurodevelopmental disorder is autism spectrum disorder (ASD). In embodiments, the neurological disorder or neurodevelopmental disorder is Alzheimer's Disease (AD). In embodiments, the present disclosure provides a method of treating essential tremor in a patient in need thereof, comprising administering a drug substance of the present disclosure to the patient. In embodiments, the present disclosure provides a method of treating essential tremor in a patient in need thereof, comprising administering a pharmaceutical composition of the present disclosure to the patient.

In embodiments, the present disclosure provides a method of reducing or ameliorating symptoms associated with a neurological or neurodevelopmental disorder in a patient in need thereof, comprising administering a drug substance of the present disclosure to the patient. In embodiments, the symptoms comprise pain, deficits in social interaction, agitation, anxiety, repetitive behaviors, irritability, aggression, hyperactivity, or a combination thereof.

In embodiments, the efficacy of treatment is determined by any one of suitable measurements known in the art (e.g., numerical rating scale score). In embodiments, the severity of the disorder during the administration period is at least about 10% (for example, at least 15%, at least 20%, at least 25%, at least 30%, at least 35%, at least 40%, at least 45, at least 50%, at least 55%, at least 60%, at least 65%, at least 70%, at least 75%, at least 80%, at least 85%, at least 90%, at least 95%, or at least 100%, including all values and subranges that lie therebetween) lower than the severity of the disorder prior to treatment. In embodiments, the patient is an adult. In embodiments, the patient is 18-65 years old. In embodiments, the patient is at least 64 years old. In embodiments, the patient is 65-95 years old.

### NUMBERED EMBODIMENTS

1. A composition characterised in that it comprises about 65 to about 95% (w/w) cannabidiol (CBD) and one or more of the following cannabinoids: trans-tetrahydrocannabinol (trans-THC); cannabichromene (CBC); cannabigerol (CBG); cannabidivarin (CBDV); cannabidiol-C4 (CBD-C4); and cis-tetrahydrocannabinol (cis-THC).
2. The composition according to embodiment 1, wherein the composition comprises about 0.4 to about 3.0% (w/w) trans-THC.
3. The composition according to embodiment 1 or 2, wherein the composition comprises about 1.0 to about 4.0% (w/w) CBC.
4. The composition according to any one of embodiments 1 to 3, wherein the composition comprises about 0.2 to about 4.0% (w/w) CBG.
5. The composition according to any one of embodiments 1 to 4, wherein the composition comprises about 0.2 to about 1.4% (w/w) CBDV.
6. The composition according to any one of embodiments 1 to 5, wherein the composition comprises about 0.1 and 0.8% (w/w) CBD-C4.
7. The composition according to any one of embodiments 1 to 6, wherein the composition comprises about 0.1 to about 1.6% (w/w) cis-THC.
8. The composition according to any one of embodiments 1 to 7, wherein the composition comprises one or more of: terpenes; triglycerides; sterols; fatty acids; and carotenoids.
9. The composition according to embodiment 8, wherein the composition comprises about 1.0 to about 3.0% (w/w) terpenes.
10. The composition according to embodiments 8 or 9, wherein the composition comprises about 0.8 and 3.0% (w/w) triglycerides.
11. The composition according to any one of embodiments 8 to 10, wherein the composition comprises about 0.5 to about 2.0% (w/w) sterols.
12. A pharmaceutical composition comprising the composition of any one of embodiments 1 to 11 and one or more additional ingredients selected from carriers, diluents (e.g. oils), excipients, adjuvants, fillers, buffers, binders, disintegrants, preservatives, antioxidants, lubricants, stabilisers, solubilisers, surfactants, masking agents, colouring agents, flavouring agents, and sweetening agents.
13. The pharmaceutical composition of embodiment 12 in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.
14. The composition according to any one of embodiments 1 to 11, or the pharmaceutical composition of embodiment 12 or embodiment 13, for use in a method of treatment.
15. The composition according to any one of embodiments 1 to 11, or the pharmaceutical composition of embodiment 12 or embodiment 13, for use in the treatment of a neurological disorder.
16. The composition according to any one of embodiments 1 to 11, or the pharmaceutical composition of embodiment 12 or embodiment 13, for use in the treatment of a neurodegenerative disorder.
17. The composition according to any one of embodiments 1 to 11, or the pharmaceutical composition of embodiment 12 or 13, for use in the treatment of non-cognitive symptoms and behaviours associated with a neurodegenerative disorder, wherein the non-cognitive symptoms and behaviours comprise one or more of the following: psychosis, agitation, aggression, irritability, depression, and/or anxiety.
18. The composition according to any one of embodiments 1 to 11, or the pharmaceutical composition of embodiment 12 or 13, for use in the treatment of Alzheimer's Disease (AD).
19. The composition according to any one of embodiments 1 to 11, or the pharmaceutical composition of embodiment 12 or 13, for use in the treatment of Essential Tremor.
20. The composition according to any one of embodiments 1 to 11, or the pharmaceutical composition of embodiment 12 or 13, for use in the treatment of non-cognitive symptoms and behaviours associated with Alzheimer's Disease (AD), wherein the non-cognitive symptoms and behaviours comprise one or more of the following: psychosis, agitation, aggression, irritability, depression, and/or anxiety.
21. The composition according to any one of embodiments 1 to 11, or the pharmaceutical composition of embodiment 12 or 13, for use in the treatment of agitation associated with Alzheimer's Disease (AD).
22. A method of treatment comprising administering to a patient in need of treatment a therapeutically effective amount of the composition of embodiments 1 to 11 or the pharmaceutical composition of embodiment 12 or 13.
23. The composition according to any one of embodiments 1 to 11, or the pharmaceutical composition of embodiment 12 or embodiment 13, for use in the treatment of symptoms associated with a neurodevelopmental disorder, wherein the symptoms comprise one or more of the following: deficits in social interaction; repetitive behaviours; irritability; aggression; and hyperactivity.
24. The composition according to any one of embodiments 1 to 11, or the pharmaceutical composition of embodiment 12 or 13, for use in the treatment of Autism Spectrum Disorder (ASD).
25. The composition according to any one of embodiments 1 to 11, or the pharmaceutical composition of embodiment 12 or 13, for use in the treatment of symptoms associated with ASD, wherein the symptoms comprise one or more of the following: deficits in social interaction; repetitive behaviours; irritability; aggression; and hyperactivity.
26. The composition according to any one of embodiments 1 to 11, or the pharmaceutical composition of embodiment 12 or 13, for use in the treatment of irritability associated with ASD.
27. The composition according to any one of embodiments 1 to 11, or the pharmaceutical composition of embodiment 12 or 13, for use in the treatment of a patient with ASD, wherein the patient is an adult.
28. The composition according to any one of embodiments 1 to 11, or the pharmaceutical composition of embodiment 12 or 13, for use in the treatment of a patient with symptoms associated with ASD, wherein the patient is an adult and the symptoms comprise one or more of the following: deficits in social interaction; repetitive behaviours; irritability; aggression; hyperactivity.
29. The composition according to any one of embodiments 1 to 11, or the pharmaceutical composition of embodiment 12 or 13, for use in the treatment of a patient with symptoms associated with ASD, wherein the patient is an adult and the symptoms comprise irritability.

### EXAMPLES

The present disclosure is further illustrated by reference to the following Examples. The Examples are illustrative and are not to be construed as restricting the scope of the invention in any way.

### Example 1. Drug Substance Extraction Process

Exemplary drug substance (DS) extraction processes are summarized in FIGS. 1A-B. The concentrations of components in DS-A and DS-B are listed in Table A.

TABLE A
Compositions A and B (Dry compositions)

| | | | | Mean − | Mean + |
|---|---|---|---|---|---|
| | Specification | DS-A | DS-B | 3SD | 3SD |
| Components | Limits | (% w/w) | (% w/w) | (% w/w) | (% w/w) |
| Total cannabinoids | 76.0-100.0 | 84.6-92.0 | 76.9-82.9 | 70.08 | 95.23 |
| CBD | 68.0-94.0 | 77.4-83.6 | 69.5-75.2 | 63.29 | 86.47 |
| CBD-C4 | 0.2-0.8 | 0.3 | 0.3-0.4 | 0.23 | 0.39 |
| CBDV | 0.2-1.4 | 0.4-0.7 | 0.6-0.7 | 0.42 | 0.83 |
| CBG | 0.2-4.0 | 0.9-1.5 | 0.5-0.6 | 0.00 | 1.56 |
| Cis-THC | 0.2-1.6 | 0.6-0.9 | 0.6-0.7 | 0.40 | 0.94 |
| Trans-THC | 0.4-3.0 | 1.8-1.9 | 1.3-1.5 | 0.76 | 2.20 |
| CBC | 1.0-4.0 | 1.7-2.3 | 1.5-1.8 | 1.21 | 2.36 |
| Total terpenes | 1.0-3.0 | 2.0-2.8 | 4.7-7.3 | 0 | 9.98 |
| Total triglycerides | 0.8-3.0 | 1.6-2.6 | N/A | | |
| Total sterols | 0.5-2.0 | 1.0-1.7 | 0.7-1.5 | 0.14 | 2.16 |

The concentrations of the components in drug substance, DS-C, DS-D, DS-E, and DS-F are shown in Table B.

TABLE B
Drug substance (DS-C, DS-D, DS-E, and DS-F) components

| Component | DS-C | | DS-D | | DS-E | | DS-F | |
|---|---|---|---|---|---|---|---|---|
| CBD | 75.3% | w/w | 73.62% | w/w | 78.06% | w/w | 69.5-83.6% | w/w |
| Trans-THC | 1.6% | w/w | 1.55% | w/w | 0.89% | w/w | 1.3-1.9% | w/w |
| CBC | 1.9% | w/w | 1.72% | w/w | 1.86% | w/w | 1.5-2.3% | w/w |
| CBG | 0.9% | w/w | 0.74% | w/w | 1.02% | w/w | 0.5-1.5% | w/w |
| CBDV | 0.4% | w/w | 0.36% | w/w | 0.63% | w/w | 0.4-0.7% | w/w |
| CBD-C4 | 0.2% | w/w | 0.20% | w/w | 0.28% | w/w | 0.3-0.4% | w/w |
| Total cannabinoids | 82.6% | w/w | 80.13% | w/w | 85.08% | w/w | 76.9-92.0% | w/w |

### Example 2: Effect of Drug Substance on Behavioural Measures with Relevance to Alzheimer's Disease

The aim of this study was to evaluate the efficacy of the present drug substance to modulate the agitation-like behaviours in the rTg4510 regulatable mouse model of Alzheimer's Disease (AD). Animals were tested in a behavioural assay, the Open Field (OF) test to measure locomotor activity (LMA). Activity was first assessed at a 21-week-old baseline to determine whether a hyperactive phenotype (to be used as a proxy for agitation) had developed and was further assessed at 2 subsequent ages in acute and chronic efficacy phases.

The rTg4510 mouse phenocopies the tau pathology and pronounced neurodegeneration observed in human tauopathies and provide temporal control over mutant tau transgene expression. The aggregation and deposition of misfolded tau protein in the forebrain of these animals is a time-dependent phenomenon and as such, the mouse model provides an opportunity to perform longitudinal analysis of the behaviours beginning at week 21 where little tau has been deposited, to week 32 where significant tau aggregation is known to have occurred and a distinct phenotype should have developed.

Wild-type control mice were also included in this study, being non carrier for both the genes in the transgenic mouse; these provided an important control for the phenotypic comparison.

A subset of transgenic mice was treated with doxycycline with the intention of switching off the tau transgene to provide another control group for this study.

The rTg4510 Mouse Model

Transgenic mice expressing mutations in amyloid precursor protein (APP) have been an essential tool in research but these mice are limited as models of Alzheimer's disease by the absence of neurofibrillary tangles and little or no neuronal loss. The P301L mutation in exon 10 is the most common mutation linked to fronto-temporal dementia and is pathologically characterised by the presence of neurofibrillary tangles and neuronal loss in the forebrain. The rTg4510 model overexpresses mutated human P301L tau driven by the calcium/calmodulin-dependent kinase II (CaMKII) promoter which designates the overexpression to the forebrain, thus allowing the study of the effects of tauP301L in the brain regions most severely affected in Alzheimer's disease.

rTg4510 mice are bitransgenic for genes Tg(Camk2a-tTA)1Mmay, containing the tetracycline-controlled transactivator protein (tTA) under regulatory control of the CaMKII promoter, and Fgf14^{Tg(tetO-MAPT*P301L)4510Kha}, this being the human four repeat-tau gene lacking the amino-terminal sequences (4R0N) containing the frontotemporal dementia-associated P301L mutation sequence. Presence of both sequences results in transgene suppression controlled by the tTA in the presence of antibiotics such as doxycycline.

rTg4510 bitransgenic mice have mutated human tau^{P301L} over-expression, approximately 13-fold greater than murine tau. This high tau^{P301L} expression results in age-independent behavioural and pathological abnormalities, as well as age-dependent functional and structural abnormalities, associated with the progression of Alzheimer's disease. Learning and memory tests performed on bitransgenic mice indicate impairments in the hippocampus and amygdala dysfunction. In addition, significant tau burden and neuronal death is observed in the hippocampus, cortex and amygdala. The behavioural and amygdala pathologies mimic the neurodegenerative tauopathy, frontotemporal dementia with parkinsonism linked to chromosome 17 (FTDP-17). After bitransgenic mice are administered doxycycline, neuronal death ceases and the ability to acquire and retain new spatial memories is restored. When maintained under doxycycline conditions, these bitransgenic mice may be expected to have the same phenotype as mice singly transgenic for tetO-MAPT*P301L: the untranslated sequence from the prion promoter results in moderate levels of tau^{P301L} expression in brain before Tet-induction, but does not result in tauopathies. Administration of doxycycline for 6-8 weeks has been demonstrated to decrease the tau mRNA to ˜15% of the maximum tau^{P301L} level.

The high levels of transgenic tau^{P301L} expression achieved induce age-dependent development of the three major pathological hallmarks of human tauopathy: 1) memory impairment, 2) NFT, and 3) neuron loss.

Hyperactivity has been reported in rTg4510 mice at 4 months of age (12% of animals) and increases up to 62% of animals>24 weeks of age. At 32 weeks of age 74% of animals are hyperactive, and when tested in simulated home-cage test exhibit an abnormal activity pattern where there is still significant activity during the light phase. This aspect is known to be the agitative-like behaviour. Changes in activity correlate with tau burden and the doxycycline-treated animals show significantly less hyperactivity when tested at 24 weeks. Previously, the hyperactive phenotype has been used as a model for agitation in AD and other dementias.

Motor function appears normal but as they age rTg4510 mice exhibit clasping and hind limb retraction when lifted and eventually display a dystonic posture with tail rigour at advanced ages. Enhanced monitoring and twice-weekly scoring was implemented in this study to track any change in hindlimb extension score. Diet pellets were presented on the floor of the cages when animals were singly housed to mitigate against weight loss caused by decreased feeding as a result of hyperactivity.

### Methods

Ninety male rTg4510 (CC) mice and 15 wild-type (WW) (Tg(Camk2a-tTA)1Mmay Fgf14^{Tg(tetO-MAPT*P301L)4510Kha}/J, Jax strain #024854, licensed from the Mayo Clinic, USA) were split into 2 cohorts, 4 weeks apart in age; cohort 1 contained 53 mice (8 WW and 45 CC) and cohort 2 contained 52 mice (7 WW and 45 CC). Relative humidity was 55±15% throughout. Mice were housed in individually ventilated cages on a 12 h/12 h light/dark cycle (lights on at 07.00 h; illumination by red light during the dark phase), at an ambient temperature of 24±4° C. with free access to standard rodent maintenance diet and filtered water. Animals were allowed to acclimatise to the above conditions for 7-10 days prior to the study start. All housing groupings were preserved until the mice were approximately 5 months old, at which point they were singly housed.

### Experimental Procedures

Microchipping: animals were microchipped subcutaneously between the shoulder blades (Pico 7 mm ISO transponders, Aston Pharma, UK).

Weekly bodyweight: all animals were weighed weekly until the end of the study.

Doxycycline administration: a subset of animals in cohort 2 (n=15) was administered 2× daily oral bolus doses of doxycycline hyclate (10 mg/kg p.o. in 5% glucose, Sigma Aldrich) at 13 weeks of age, followed by continuous exposure to normal lab chow containing 200 ppm doxycycline (Envigo Teklad 2018 TD.00502 no colourants, irradiated and vacuum-packed). All other animals received Envigo Teklad 2018 diet. All diet was provided ad libitum.

Singly housing: all animals were singly housed from approximately 5 months of age until the end of the study.

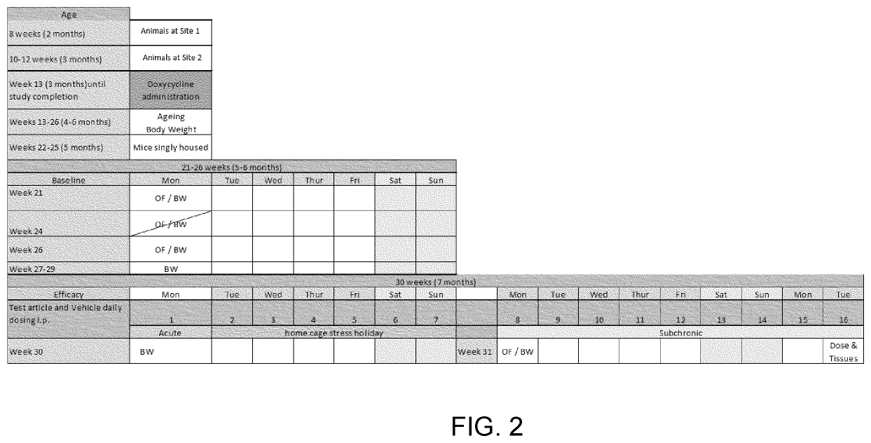

Animals were administered vehicle, Risperidone (0.125 mg/kg i.p.) or DS-C as described in Example 1 (10, 30 or 100 mg/kg/day i.p.) according to the experimental design (FIG. 2 and Table C). Following this regime, animals were evaluated in Open Field (OF) test, before being deeply anaesthetised at the end of the behavioural testing prior to biofluid sampling and tissue harvesting.

Brain Tissue Collection: Mice were decapitated and the brains rapidly extracted. Brains were briefly washed in saline and blot dried then hemisected, weighed separately and frozen on dry ice before storage at approximately −80° C.

TABLE C
Sample size per group, doses and pre-treatment times.

| | | | Treatment i.p. | |
|---|---|---|---|---|
| Group | Genotype | Diet | (30 or 60 minute pretreatment) | n |
| A | rTG4510 WW | Teklad 2018 | Vehicle | 15 |
| B | rTg4510 CC | Teklad 2018 | Vehicle | 15 |
| C | rTg4510 CC | Doxycycline 200 ppm | Vehicle | 15 |
| | | (Teklad 2018) | | |
| D | rTg4510 CC | Teklad 2018 | Risperidone (0.125 mg/kg) | 15 |
| E | rTg4510 CC | Teklad 2018 | DS (10 mg/kg) | 15 |
| F | rTg4510 CC | Teklad 2018 | DS (30 mg/kg) | 15 |
| G | rTg4510 CC | Teklad 2018 | DS (100 mg/kg) | 15 |

### Drugs

Risperidone: Risperidone was purchased from Glentham Life Sciences (Product Code GP4047; Batch 517XDR). Risperidone was dosed via the intraperitoneal route as a clear solution at a dose volume of 10 mL/kg in a vehicle of acidified saline. Specifically, the material was solubilised by the addition of 50 μL of 10% glacial acetic acid and making up to volume with 0.9% saline (Aquapharm, Animal Care, York, UK, Lot 21030311).

Drug Substance (DS): (i.e., DS-C, See Table B in Example 1) Dosing volume was 10 mL/kg and DS was formulated at the 150 mg/kg dose in a vehicle of ethanol: Kolliphor HS15: 0.9% saline (1:12:87, w/w/v) which were stored at room temperature and added at approximately 60° C. to form solutions which were subsequently stable at room temperature. All dosing formulations were protected from light whenever possible. Dosing solutions were made fresh on the day of dosing, with lower dose formulations being prepared by dilution from the 150 mg/kg dosing solution at room temperature.

### Detailed Methods

### Behavioural Testing

Locomotor activity was assessed by OF test in the light phase of the light-dark cycle at weeks 21, 26 and 31. An automated beam break apparatus (40 cages) was used for the assessment of locomotor activity in rats and mice. Each cage had 4 beams; 1 ‘y’ and 3 ‘x’ which triggered a signal through capacitors when the beam was broken. Signals were collected by data loggers and processed accordingly. All animals were placed in the apparatus and spontaneous activity was assessed for a four-hour period between 0900 and 1300. Animals were then removed from the apparatus and returned to their home cages. LMA assessment was split over 2 consecutive days at each time point.

### Statistical Methods

Data were analysed using SAS statistical data analysis software v9.4 (SAS Institute) and R 4.1.2 using package e1071 v 1.7-9 (the R Foundation).

Weekly body weight data were analysed by mixed linear model with the cohort by week and group by week interactions as fixed effects, animal as the random subject, using an AR (1)+RE correlation structure. Comparisons to the WW and rTg4510 (vehicle) groups for the same week were by the multiple t test.

Brain weights were analysed by 2-way analysis of variance with treatment and cohort as factors. As brain weight was found to be correlated with body weight and there were differences between the groups in terminal body weight, effects on brain weight in addition to what would be expected, given the effects on body weight, were determined by two-way analysis of covariance with terminal (week 32) body weight as the covariate.

For locomotor data, a log (x+1) transformation was used. For weeks 21 & 26, means and SEMs were calculated from the transformed data. Analysis was by Friedman's two-way nonparametric ANOVA with treatment and test day as factors to compare each group to the WW animals, and (where appropriate) Doxycycline with the untreated rTg4510 group. For week 30, data excluding the WW and Doxycycline groups for each time period were analysed by robust regression model using M estimation, Huber weighting, using the default parameter c=1.345. The model included treatment and Day as factors and the 0-60 activity from Week 26 as a covariate.

For comparisons involving the WW and Doxycycline groups, the model with treatment and Day as factors and no covariate was used.

### Results

### General Observations

All animals were in good condition throughout the early arrival and baseline phases of the study. Mice gained weight according to expectations.

The initial schedule incorporated open field testing for locomotor activity at week 24 and not week 26. No data were collected at week 24 for cohort 1 owing to equipment failure and so a re-test was performed at week 26.

Brain weight can be used as an indication of the genotype of the animal since there is strong evidence that rTg4510 CC animals have smaller brains than their WW counterparts because of the forebrain degeneration observed at later ages. Age matched controls exhibit a decrease in whole-brain weight from as early as 1 month.

Brain weights from animals which completed the study were as follows: mean brain weight was 456.8±5.3 mg for rTg4510 WW and 383.7±6.0 mg for rTg4510 CC mice which received vehicle (p<0.001 vs WW mice).

Administration of doxycycline to rTg4510 CC mice elicited a change in mean brain weight to 408.8±6.1 mg (p<0.001 vs WW mice; p=0.001 vs rTg4510 CC mice).

There were no significant differences in brain weight in rTg4510 CC mice receiving risperidone or DS when compared with rTg4510 CC mice which received vehicle.

### Body Weight and the Effect of Doxycycline

Mice were weighed on a weekly basis. Wild-type animals were consistently and significantly heavier than their transgenic counterparts in both cohorts; WW 28.67±0.26 g vs rTg4510 25.05±0.12 g at 8 weeks of age (p<0.001 vs WW), through to WW 43.60±0.6 g vs rTg4510 29.98±1.01 g at 30 weeks of age (p<0.001 vs WW). Doxycycline administration in the diet from 13 weeks of age ameliorated the difference in body weight within 2 weeks of starting the doxycycline regime. Animals fed doxycycline were significantly heavier than rTg4510 from week 15 through to completion of the study; WW 34.40±0.38 g vs rTg4510 28.33±0.21 g vs Doxycycline 29.14±0.50 g (p<0.05 vs rTg4510) at 15 weeks of age.

Animals were administered Risperidone or DS in acute and chronic efficacy phases of the study (weeks 30-32). There were no significant changes in bodyweight in the efficacy parts of the study.

### Longitudinal Assessment of Locomotor Activity

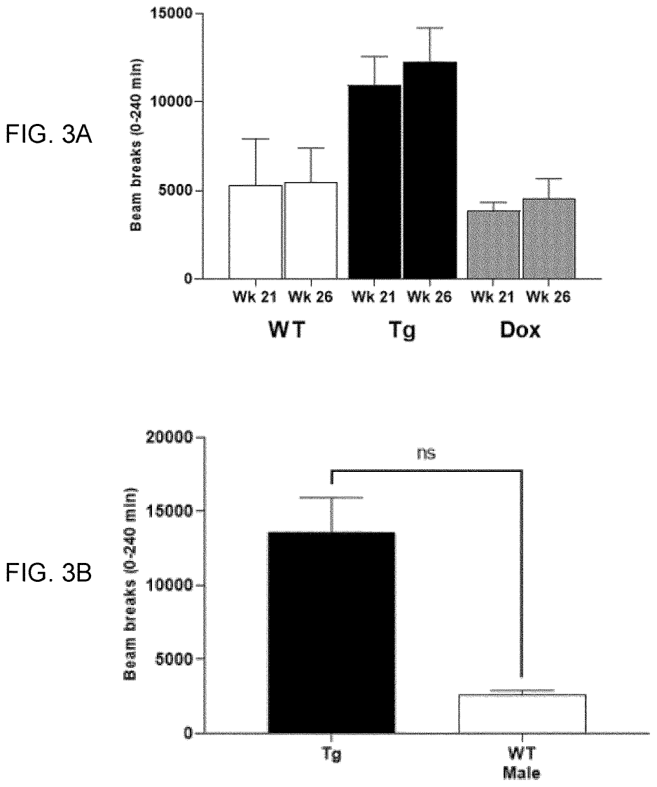

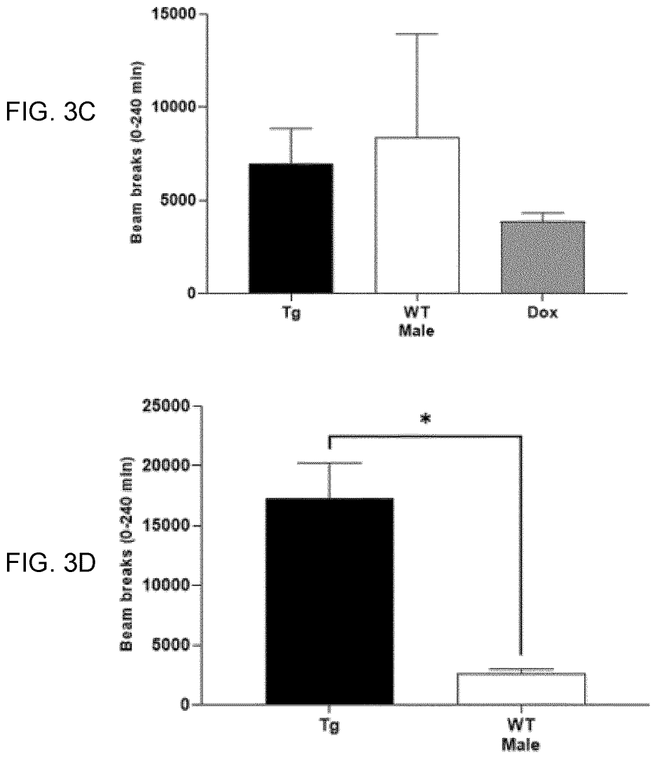

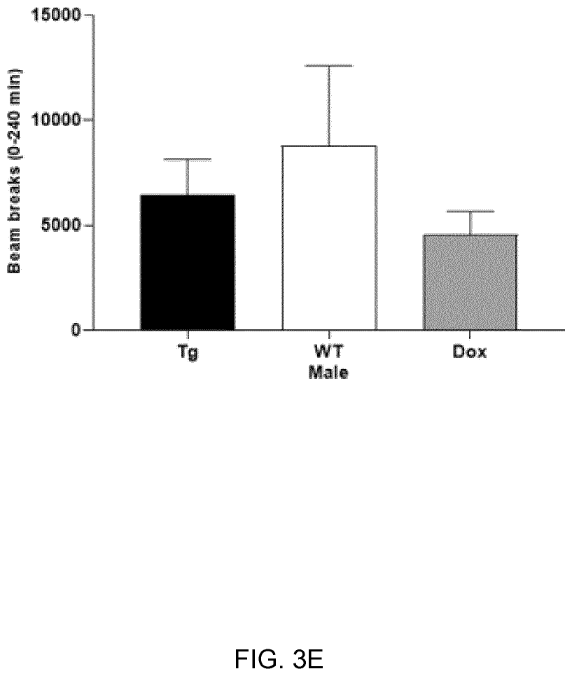

The longitudinal assessment of locomotor activity at 21 and 26 weeks of age is shown in FIGS. 3A-E and Tables D and E. At 21 weeks of age there was a marked increase in locomotor activity in the rTg4510 group compared to both the WW and doxycycline-treated animals when considering both cohorts (FIG. 3A). The effects of genotype were very clear in cohort 1 (FIG. 3B) and the pattern of activity in cohort 2 was driven by one WW animal which was significantly more active than the rest of the group, FIG. 3C. A similar pattern of hyperlocomotion was seen at 26 weeks of age, although in cohort 1 the hyperactivity seen in the rTg4510 animals was now significantly different from WW animals (FIG. 3D, p<0.05 0-240 minutes). In cohort 2 the hyperlocomotion observed in the WW group was driven by 2 animals (FIG. 3E). There is no evidence that these animals were mis-genotyped, the brain- and body weights are within the limits for the groups and so all figures are constructed with these animals included in the analysis.

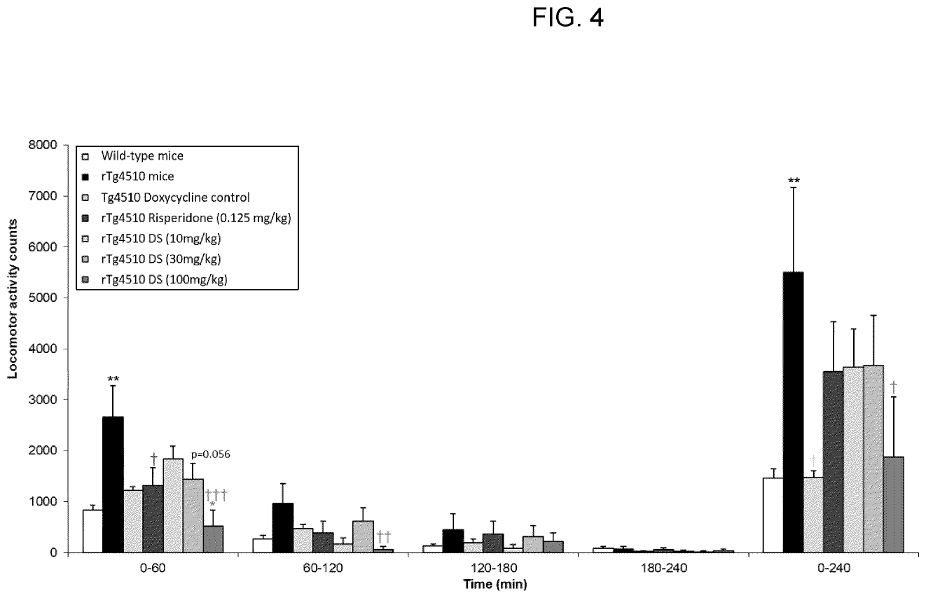

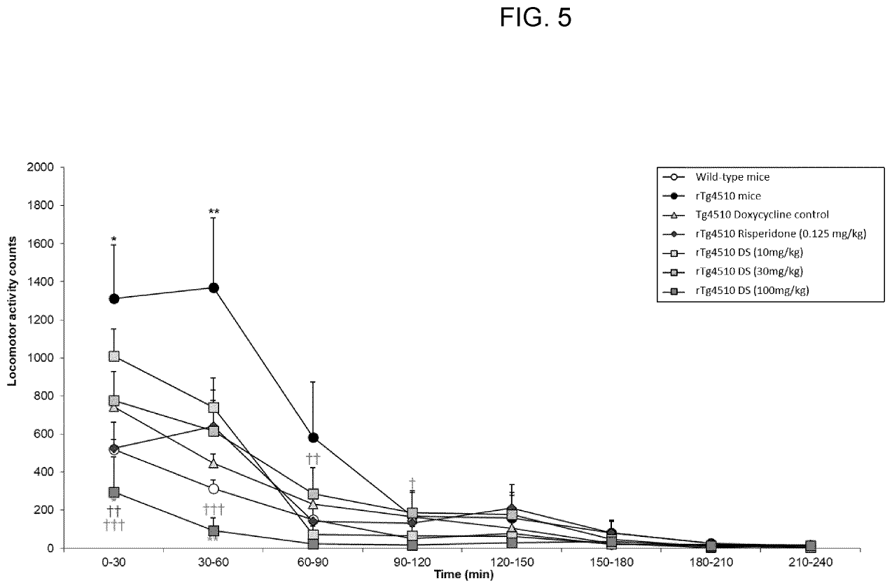

The effect of administration of DS (10-100 mg/kg/d×8 days) to rTg4510 mice on locomotor activity (Cohorts 1 and 2, Week 31) is shown in FIGS. 4-5 and Table F. rTg4510 CC mice administered with vehicle exhibited a significant hyperlocomotor phenotype compared to wild-type animals (p<0.01 0-60 minutes and 0-240 minutes). Activity in the rTg4510_{(vehicle)} group was 383.3% of the activity in the WW group across the whole test period (0-240 minutes). This hyperlocomotion was significantly reversed by risperidone (0.125 mg/kg/d) treatment at 0-60 minutes (p<0.05 vs rTg4510_{(vehicle)}) (FIG. 4 and Table F).

Animals which had been treated with doxycycline showed decreased locomotor activity compared to vehicle-treated rTg4510 animals (p<0.05 vs rTg4510_{(vehicle)}, 0-240 minutes). Activity in the doxycycline-treated group was 100.6% of the activity seen in the WW animals and 26.2% of the activity seen in the rTg4510_{(vehicle)} animals for the 0-240 minute period (FIG. 4 and Table F).

Administration of DS (100 mg/kg/d) to rTg4510 mice decreased the hyperlocomotor activity when compared to both WW (p<0.05, 0-60 minutes) and vehicle-treated rTg4510 mice (p<0.001 vs rTg4510_{(vehicle)} 0-60 minutes, p<0.01 vs rTg4510_{(vehicle)} 60-120 minutes, p<0.05 vs rTg4510_{(vehicle)} 0-240 minutes, FIGS. 4-5 and Table F).

TABLE D
Locomotor activity totals Cohort 1 & 2 Week 21

| | | | | | |
|---|---|---|---|---|---|
| | | | | p v | p v |
| Group | n | Mean | SEM | Wild-type | rTg4510 |

0-30 minutes

| | | | | | |
|---|---|---|---|---|---|
| Wild-type | 15 | 930.1 | 107.7 | | |
| rTg4510 | 75 | 1218.0 | 111.0 | 0.976 | |
| rTg4510 Doxycycline | 15 | 855.0 | 44.3 | 0.637 | 0.601 |

30-60 minutes

| | | | | | |
|---|---|---|---|---|---|
| Wild-type | 15 | 741.7 | 112.9 | | |
| rTg4510 | 75 | 1180.6 | 130.7 | 0.146 | |
| rTg4510 Doxycycline | 15 | 749.9 | 86.7 | 0.360 | 0.959 |

60-90 minutes

| | | | | | |
|---|---|---|---|---|---|
| Wild-type | 15 | 458.7 | 106.0 | | |
| rTg4510 | 75 | 757.3 | 112.2 | 0.171 | |
| rTg4510 Doxycycline | 15 | 542.2 | 71.4 | 0.151 | 0.570 |

90-120 minutes

| | | | | | |
|---|---|---|---|---|---|
| Wild-type | 15 | 182.6 | 73.7 | | |
| rTg4510 | 75 | 291.6 | 69.7 | 0.331 | |
| rTg4510 Doxycycline | 15 | 283.2 | 99.0 | 0.223 | 0.537 |

120-150 minutes

| | | | | | |
|---|---|---|---|---|---|
| Wild-type | 15 | 177.2 | 64.4 | | |
| rTg4510 | 75 | 215.8 | 55.6 | 0.763 | |
| rTg4510 Doxycycline | 15 | 227.4 | 60.0 | 0.208 | 0.258 |

150-180 minutes

| | | | | | |
|---|---|---|---|---|---|
| Wild-type | 15 | 57.3 | 33.6 | | |
| rTg4510 | 75 | 175.7 | 50.1 | 0.066 | |
| rTg4510 Doxycycline | 15 | 181.8 | 87.2 | 0.111 | 0.687 |

180-210 minutes

| | | | | | |
|---|---|---|---|---|---|
| Wild-type | 15 | 23.9 | 17.7 | | |
| rTg4510 | 75 | 90.3 | 28.4 | 0.219 | |
| rTg4510 Doxycycline | 15 | 71.1 | 36.8 | 0.176 | 0.562 |

210-240 minutes

| | | | | | |
|---|---|---|---|---|---|
| Wild-type | 15 | 22.8 | 17.5 | | |
| rTg4510 | 75 | 65.2 | 21.0 | 0.355 | |
| rTg4510 Doxycycline | 15 | 52.4 | 27.5 | 0.646 | 0.870 |

0-60 minutes

| | | | | | |
|---|---|---|---|---|---|
| Wild-type | 15 | 1684.0 | 218.9 | | |
| rTg4510 | 75 | 2420.8 | 242.0 | 0.432 | |
| rTg4510 Doxycycline | 15 | 1624.9 | 121.7 | 0.623 | 0.977 |

60-120 minutes

| | | | | | |
|---|---|---|---|---|---|
| Wild-type | 15 | 673.3 | 179.8 | | |
| rTg4510 | 75 | 1191.3 | 189.2 | 0.171 | |
| rTg4510 Doxycycline | 15 | 880.1 | 153.6 | 0.100 | 0.427 |

120-180 minutes

| | | | | | |
|---|---|---|---|---|---|
| Wild-type | 15 | 312.1 | 101.0 | | |
| rTg4510 | 75 | 541.6 | 120.7 | 0.205 | |
| rTg4510 Doxycycline | 15 | 521.1 | 130.2 | 0.105 | 0.399 |

180-240 minutes

| | | | | | |
|---|---|---|---|---|---|
| Wild-type | 15 | 72.0 | 51.1 | | |
| rTg4510 | 75 | 207.7 | 59.9 | 0.342 | |
| rTg4510 Doxycycline | 15 | 212.8 | 58.8 | 0.484 | 0.938 |

0-240 minutes

| | | | | | |
|---|---|---|---|---|---|
| Wild-type | 15 | 3106.7 | 619.4 | | |
| rTg4510 | 75 | 5285.3 | 700.7 | 0.177 | |
| rTg4510 Doxycycline | 15 | 3476.4 | 430.0 | 0.117 | 0.469 |

Means and SEMs are back-transformed.
Comparisons to rTg4510 and Wild-type mice were by Friedman's test.

TABLE E
Locomotor activity totals Cohort 1 & 2 Week 26

| | | | | | |
|---|---|---|---|---|---|
| | | | | p v | p v |
| Group | n | Mean | SEM | Wild-type | rTg4510 |

0-30 minutes

| | | | | | |
|---|---|---|---|---|---|
| Wild-type | 15 | 941.5 | 150.7 | | |
| rTg4510 | 75 | 1312.8 | 124.5 | 0.278 | |
| rTg4510 Doxycycline | 15 | 903.7 | 82.6 | 0.631 | 0.800 |

30-60 minutes

| | | | | | |
|---|---|---|---|---|---|
| Wild-type | 15 | 740.2 | 152.4 | | |
| rTg4510 | 75 | 1122.3 | 146.3 | 0.368 | |
| rTg4510 Doxycycline | 15 | 664.6 | 125.1 | 0.328 | 0.681 |

60-90 minutes

| | | | | | |
|---|---|---|---|---|---|
| Wild-type | 15 | 492.1 | 129.3 | | |
| rTg4510 | 65 | 629.9 | 139.5 | 0.478 | |
| rTg4510 Doxycycline | 15 | 489.8 | 109.3 | 0.312 | 0.565 |

90-120 minutes

| | | | | | |
|---|---|---|---|---|---|
| Wild-type | 15 | 187.7 | 92.5 | | |
| rTg4510 | 65 | 370.2 | 98.6 | 0.239 | |
| rTg4510 Doxycycline | 15 | 186.7 | 91.1 | 0.358 | 0.888 |

120-150 minutes

| | | | | | |
|---|---|---|---|---|---|
| Wild-type | 15 | 121.5 | 62.6 | | |
| rTg4510 | 65 | 247.2 | 73.3 | 0.285 | |
| rTg4510 Doxycycline | 15 | 157.0 | 67.8 | 0.776 | 0.644 |

150-180 minutes

| | | | | | |
|---|---|---|---|---|---|
| Wild-type | 15 | 117.1 | 53.6 | | |
| rTg4510 | 65 | 202.2 | 63.1 | 0.592 | |
| rTg4510 Doxycycline | 15 | 142.2 | 69.0 | 0.974 | 0.728 |

180-210 minutes

| | | | | | |
|---|---|---|---|---|---|
| Wild-type | 15 | 81.5 | 46.5 | | |
| rTg4510 | 75 | 112.3 | 36.2 | 0.720 | |
| rTg4510 Doxycycline | 15 | 185.3 | 82.8 | 0.107 | 0.142 |

210-240 minutes

| | | | | | |
|---|---|---|---|---|---|
| Wild-type | 15 | 51.5 | 35.1 | | |
| rTg4510 | 75 | 80.7 | 24.4 | 0.767 | |
| rTg4510 Doxycycline | 15 | 41.3 | 27.6 | 0.705 | 0.846 |

0-60 minutes

| | | | | | |
|---|---|---|---|---|---|
| Wild-type | 15 | 1690.6 | 306.4 | | |
| rTg4510 | 75 | 2494.8 | 270.2 | 0.366 | |
| rTg4510 Doxycycline | 15 | 1606.6 | 192.7 | 0.440 | 0.851 |

60-120 minutes

| | | | | | |
|---|---|---|---|---|---|
| Wild-type | 15 | 759.8 | 225.1 | | |
| rTg4510 | 65 | 1173.2 | 241.9 | 0.306 | |
| rTg4510 Doxycycline | 15 | 770.3 | 193.6 | 0.213 | 0.547 |

120-180 minutes

| | | | | | |
|---|---|---|---|---|---|
| Wild-type | 15 | 330.6 | 120.3 | | |
| rTg4510 | 65 | 624.6 | 156.7 | 0.323 | |
| rTg4510 Doxycycline | 15 | 459.9 | 118.1 | 0.803 | 0.660 |

180-240 minutes

| | | | | | |
|---|---|---|---|---|---|
| Wild-type | 15 | 244.8 | 96.9 | | |
| rTg4510 | 75 | 340.7 | 85.7 | 0.588 | |
| rTg4510 Doxycycline | 15 | 362.6 | 106.3 | 0.134 | 0.222 |

0-240 minutes

| | | | | | |
|---|---|---|---|---|---|
| Wild-type | 15 | 3389.1 | 766.6 | | |
| rTg4510 | 65 | 5658.5 | 874.1 | 0.589 | |
| rTg4510 Doxycycline | 15 | 3435.5 | 609.3 | 0.300 | 0.469 |

Means and SEMs are back-transformed.
Comparisons to rTg4510 and Wild-type mice were by Friedman's test.

TABLE F
Locomotor activity totals Cohorts 1 & 2 Week 31

| | | | | vs Wild-type | | vs rTg4510 | |
|---|---|---|---|---|---|---|---|
| Group | n | Mean | SEM | % | p | % | p |

0-30 minutes

| | | | | | | | |
|---|---|---|---|---|---|---|---|
| Wild-type Vehicle | 15 | 519.59 | 50.92 | | | | |
| rTg4510 Vehicle | 15 | 1312.37 | 282.25 | 244.2 | 0.014* | | |
| rTg4510 Doxycycline | 15 | 744.04 | 46.92 | 143.2 | 0.415 | 58.7 | 0.255 |
| rTg4510 Risperidone (0.125 | 15 | 525.99 | 134.93 | 95.7 | 0.901 | 40.1 | 0.009** |
| mg/kg) | | | | | | | |
| rTg4510 DS (10 mg/kg) | 15 | 1008.42 | 141.81 | 194.1 | 0.155 | 76.8 | 0.439 |
| rTg4510 DS (30 mg/kg) | 15 | 776.93 | 152.30 | 136.7 | 0.708 | 59.2 | 0.147 |
| rTg4510 DS (100 mg/kg) | 15 | 296.87 | 182.90 | 41.1 | 0.036* | 22.6 | <0.001*** |

30-60 minutes

| | | | | | | | |
|---|---|---|---|---|---|---|---|
| Wild-type Vehicle | 15 | 314.49 | 46.31 | | | | |
| rTg4510 Vehicle | 15 | 1369.94 | 365.48 | 401.0 | 0.009** | | |
| rTg4510 Doxycycline | 15 | 445.74 | 49.68 | 141.7 | 0.590 | 35.3 | 0.132 |
| rTg4510 Risperidone (0.125 | 15 | 641.92 | 189.96 | 183.6 | 0.246 | 46.9 | 0.100 |
| mg/kg) | | | | | | | |
| rTg4510 DS (10 mg/kg) | 15 | 740.70 | 153.11 | 273.7 | 0.136 | 54.1 | 0.182 |
| rTg4510 DS (30 mg/kg) | 15 | 616.83 | 160.65 | 196.9 | 0.424 | 45.0 | 0.098 |
| rTg4510 DS (100 mg/kg) | 15 | 94.18 | 67.02 | 16.4 | 0.002** | 6.9 | <0.001*** |

60-90 minutes

| | | | | | | | |
|---|---|---|---|---|---|---|---|
| Wild-type Vehicle | 15 | 151.07 | 68.82 | | | | |
| rTg4510 Vehicle | 15 | 582.51 | 291.11 | 414.3 | 0.114 | | |
| rTg4510 Doxycycline | 15 | 231.53 | 45.02 | 153.3 | 0.700 | 37.0 | 0.398 |
| rTg4510 Risperidone (0.125 | 15 | 140.56 | 90.69 | 104.9 | 0.957 | 24.1 | 0.168 |
| mg/kg) | | | | | | | |
| rTg4510 DS (10 mg/kg) | 15 | 71.94 | 47.00 | 39.7 | 0.603 | 12.4 | 0.208 |
| rTg4510 DS (30 mg/kg) | 15 | 287.22 | 135.98 | 204.8 | 0.760 | 49.3 | 0.208 |
| rTg4510 DS (100 mg/kg) | 15 | 24.14 | 18.81 | 16.9 | 0.128 | 4.1 | 0.003** |

90-120 minutes

| | | | | | | | |
|---|---|---|---|---|---|---|---|
| Wild-type Vehicle | 15 | 50.97 | 28.23 | | | | |
| rTg4510 Vehicle | 15 | 167.42 | 126.31 | 292.7 | 0.313 | | |
| rTg4510 Doxycycline | 15 | 165.38 | 32.97 | 324.4 | 0.371 | 110.8 | 0.941 |
| rTg4510 Risperidone (0.125 | 15 | 132.22 | 74.03 | 241.3 | 0.408 | 79.0 | 0.826 |
| mg/kg) | | | | | | | |
| rTg4510 DS (10 mg/kg) | 15 | 65.28 | 49.63 | 92.1 | 1.000 | 39.0 | 0.834 |
| rTg4510 DS (30 mg/kg) | 15 | 187.87 | 113.67 | 382.1 | 0.445 | 112.2 | 0.834 |
| rTg4510 DS (100 mg/kg) | 15 | 17.07 | 11.95 | 27.5 | 0.494 | 10.2 | 0.048* |

120-150 minutes

| | | | | | | | |
|---|---|---|---|---|---|---|---|
| Wild-type Vehicle | 15 | 78.03 | 32.62 | | | | |
| rTg4510 Vehicle | 15 | 161.13 | 116.48 | 213.6 | 0.482 | | |
| rTg4510 Doxycycline | 15 | 105.17 | 39.26 | 134.8 | 0.823 | 63.1 | 0.745 |
| rTg4510 Risperidone (0.125 | 15 | 210.31 | 125.00 | 222.8 | 0.458 | 130.5 | 0.817 |
| mg/kg) | | | | | | | |
| rTg4510 DS (10 mg/kg) | 15 | 62.09 | 46.75 | 62.5 | 0.948 | 38.5 | 0.843 |
| rTg4510 DS (30 mg/kg) | 15 | 176.52 | 116.79 | 176.5 | 0.913 | 109.5 | 0.843 |
| rTg4510 DS (100 mg/kg) | 15 | 29.72 | 24.42 | 28.1 | 0.511 | 18.4 | 0.188 |

150-180 minutes

| | | | | | | | |
|---|---|---|---|---|---|---|---|
| Wild-type Vehicle | 15 | 20.85 | 11.90 | | | | |
| rTg4510 Vehicle | 15 | 79.90 | 67.71 | 397.9 | 0.219 | | |
| rTg4510 Doxycycline | 15 | 28.13 | 13.86 | 135.0 | 0.831 | 33.9 | 0.461 |
| rTg4510 Risperidone (0.125 | 15 | 82.39 | 60.58 | 399.5 | 0.218 | 103.1 | 0.979 |
| mg/kg) | | | | | | | |
| rTg4510 DS (10 mg/kg) | 15 | 22.37 | 18.18 | 116.5 | 0.998 | 28.0 | 0.587 |
| rTg4510 DS (30 mg/kg) | 15 | 49.04 | 31.62 | 247.7 | 0.757 | 61.4 | 0.587 |
| rTg4510 DS (100 mg/kg) | 15 | 34.75 | 28.83 | 171.2 | 0.933 | 43.5 | 0.587 |

180-210 minutes

| | | | | | | | |
|---|---|---|---|---|---|---|---|
| Wild-type Vehicle | 15 | 19.36 | 10.84 | | | | |
| rTg4510 Vehicle | 15 | 26.93 | 21.81 | 157.8 | 0.692 | | |
| rTg4510 Doxycycline | 15 | 1.72 | 1.39 | 8.9 | 0.144 | 5.6 | 0.094 |
| rTg4510 Risperidone (0.125 | 15 | 27.84 | 21.48 | 136.3 | 0.789 | 103.4 | 0.979 |
| mg/kg) | | | | | | | |
| rTg4510 DS (10 mg/kg) | 15 | 10.29 | 9.43 | 54.7 | 0.919 | 38.2 | 0.594 |
| rTg4510 DS (30 mg/kg) | 15 | 7.60 | 6.20 | 42.4 | 0.814 | 28.2 | 0.594 |
| rTg4510 DS (100 mg/kg) | 15 | 14.33 | 12.65 | 88.5 | 0.999 | 53.2 | 0.594 |

210-240 minutes

| | | | | | | | |
|---|---|---|---|---|---|---|---|
| Wild-type Vehicle | 15 | 17.81 | 12.49 | | | | |
| rTg4510 Vehicle | 15 | 12.41 | 9.69 | 77.3 | 0.817 | | |
| rTg4510 Doxycycline | 15 | 16.18 | 10.51 | 90.8 | 0.944 | 117.5 | 0.912 |
| rTg4510 Risperidone (0.125 | 15 | 6.99 | 5.48 | 37.5 | 0.392 | 56.3 | 0.629 |
| mg/kg) | | | | | | | |
| rTg4510 DS (10 mg/kg) | 15 | 7.02 | 6.05 | 46.3 | 0.833 | 56.5 | 0.990 |
| rTg4510 DS (30 mg/kg) | 15 | 6.56 | 5.91 | 37.7 | 0.725 | 52.8 | 0.990 |
| rTg4510 DS (100 mg/kg) | 15 | 15.55 | 11.28 | 102.7 | 1.000 | 125.3 | 0.990 |

0-60 minutes

| | | | | | | | |
|---|---|---|---|---|---|---|---|
| Wild-type Vehicle | 15 | 840.61 | 95.45 | | | | |
| rTg4510 Vehicle | 15 | 2656.66 | 618.64 | 304.1 | 0.007** | | |
| rTg4510 Doxycycline | 15 | 1216.28 | 80.73 | 144.7 | 0.457 | 47.6 | 0.161 |
| rTg4510 Risperidone (0.125 | 15 | 1313.59 | 356.67 | 135.0 | 0.455 | 49.4 | 0.024* |
| mg/kg) | | | | | | | |
| rTg4510 DS (10 mg/kg) | 15 | 1836.22 | 258.41 | 232.5 | 0.093 | 69.1 | 0.231 |
| rTg4510 DS (30 mg/kg) | 15 | 1441.55 | 308.91 | 165.8 | 0.448 | 54.3 | 0.056 |
| rTg4510 DS (100 mg/kg) | 15 | 516.86 | 323.12 | 37.1 | 0.038* | 19.5 | <0.001*** |

60-120 minutes

| | | | | | | | |
|---|---|---|---|---|---|---|---|
| Wild-type Vehicle | 15 | 270.25 | 71.44 | | | | |
| rTg4510 Vehicle | 15 | 966.98 | 394.14 | 357.7 | 0.163 | | |
| rTg4510 Doxycycline | 15 | 477.89 | 81.62 | 176.8 | 0.612 | 49.4 | 0.555 |
| rTg4510 Risperidone (0.125 | 15 | 384.49 | 229.85 | 134.8 | 0.743 | 39.8 | 0.314 |
| mg/kg) | | | | | | | |
| rTg4510 DS (10 mg/kg) | 15 | 174.21 | 115.63 | 47.1 | 0.741 | 18.0 | 0.285 |
| rTg4510 DS (30 mg/kg) | 15 | 618.74 | 265.90 | 202.7 | 0.775 | 64.0 | 0.285 |
| rTg4510 DS (100 mg/kg) | 15 | 67.09 | 50.30 | 19.3 | 0.177 | 6.9 | 0.005** |

Means and SEMs are back-transformed. Those for Wild-type and Doxycycline groups and comparisons involving these groups are from the analysis with treatment and cohort as factors. Those for other groups and comparisons to rTg4510 (except Doxycycline) are from the analysis with treatment and cohort as factors and Week 26 activity as a covariate. DS was compared to Wild-type by Dunnett's test and to rTg4510 by Williams' test. Other comparisons by the multiple t test.
*p < 0.05,
**p < 0.01,
***p < 0.001

### Discussions

rTg4510 Mice

The rTg4510 mice used in this study developed body- and brain-weight changes according to the inherent characteristics of the mice, confirming the genotype. The tau-dependent measures were also observed.

### Behavioural Testing

### Hyperlocomotion

rTg4510 mice are known to develop a hyperlocomotor phenotype as they age. This non-mnemonic deficit has been postulated to be a correlate to agitation.

In the present study the rTg4510 CC mice were more active than the WW control group; this is most clearly exemplified in cohort 1, which showed a non-significant difference between treatment groups at 21 weeks but a significant difference between treatment groups at 26 weeks. Administration of doxycycline returned the increased locomotor levels back to levels similar to those seen in WW mice.

There was a wide range of beam breaks within the rTg4510 group but in general a hyperactive rTg4510 animal remained hyperactive throughout the progression of the study.

It was surprisingly found that administration of DS to rTg4510 mice significantly decreased the hyperlocomotor activity when dosed at 100 mg/kg/d when compared to both WW and vehicle-treated rTg4510 mice.

### Conclusions

rTg4510 mice were used as an experimental model of behavioural and psychological symptoms of dementia. Good correlation was obtained against expectations for body- and brain-weights in that wild-type mice had higher body- and brain-weights compared to mice carrying both transgenes.

Doxycycline administration reversed the time-dependent changes in body- and brain-weight.

A hyperactivity phenotype emerged in rTg4510 mice and progressed between 21-30 weeks of age. rTg4510 transgenic mice exhibited increased levels of locomotor activity at all ages tested compared to rTg4510 wild-type mice. Statistically significant changes were observed at 31 weeks of age (p<0.01 vs rTg4510 wild-type mice, 0-240 minutes).

Doxycycline administration ameliorated the hyperactivity seen in rTg4510 mice at all ages tested; levels of locomotor activity measured in doxycycline-treated rTg4510 mice were more similar to those measured in rTg4510 wild-type mice. Statistically significant alterations in locomotor activity were evident at 31 weeks (p<0.05 vs rTg4510 transgenic mice, 0-240 minutes).

Administration of DS (100 mg/kg/d) significantly decreased locomotor activity in 31-week-old rTg4510 transgenic mice. Levels of locomotor activity were decreased compared to both rTg4510 wild-type mice (p<0.05, 0-60 minutes) and vehicle-treated rTg4510 transgenic mice (p<0.001, 0-60 minutes; p<0.01, 60-120 minutes; p<0.05 0-240 minutes).

Thus, the complex botanical mixture of the present invention was surprisingly shown to have significant effect in treating agitation in a mouse model of agitation in Alzheimer's Disease.

### Example 3: Effect of Drug Substance with Relevance to Essential Tremor

Animal models of tremor have been widely used in experimental neurology, in order to understand the pathophysiology of human tremor disorders and the development of new therapeutic agents. There are several approaches to produce tremor in animals, application of tremorgenic drugs, experimental central nervous system lesions, study of genetic mutants.

Harmaline induced essential tremor (ET) is the most widely studied animal model of ET, which shares some features with the Human essential tremor/enhanced physiological tremor for example both are associated with action tremor of similar frequency (human 4-12 Hz, rats 8-12 Hz) and both have similar responses to some pharmacological agents.

In the present study the effects of drug substance (30, 100 and 150 mg/kg i.p.) were investigated in a harmaline model of Essential Tremor.

### Methods

### Animals

All experiments were carried out in male Sprague Dawley rats (˜250 g). Naïve rats were acclimatised to the procedure room in their home cages up to 7 days following arrival at the test facility, with food and water available ad libitum. All animals were weighed at the beginning of the study and assigned to treatment groups (Vehicle n=6, treatment groups n=12/group).

### Study Protocol

Animals were dosed and tested as per Table G below, across 3 test days. Treatment groups were equally allocated across the 3 days of testing. The animals received either vehicle, DS at 30, 100 or 150 mg/kg (1 mL/kg, i.p.) or propranolol at 40 mg/kg (5 mL/kg, i.p.) (Compound 1), the animals were then dosed with vehicle or Harmaline at 10 mg/kg (5 mL/kg, i.p.) (Compound 2). The animals received DS at 30, 100 or 150 mg/kg (1 mL/kg, i.p.) 40 or 100 min before harmaline, with the first test point 20 mins later to give a pre-treatment time at first test point of 60 and 120 mins. Propanolol was dosed 30 min before harmaline and tested 20, 40 and 60 mins post-harmaline. At all test points post-harmaline, the animals were observed and given a tremor score of 0-4 (as per Table H).

TABLE G
Experimental details

| | Dosing and test time (mins) | | | | |
|---|---|---|---|---|---|
| | Veh or | Veh or | Test at | Test at | Test at |
| | Prop or DS | Harm | 20 mins | 40 mins | 60 mins |
| Veh/Veh | 0 | 30 | 50 | 70 | 90 |
| Veh/Harm | 0 | 30 | 50 | 70 | 90 |
| Prop/Harm | 0 | 30 | 50 | 70 | 90 |
| 30 mg/kg | 0 | 40 | 60 | 80 | 100 |
| DS/Harm | | | | | |
| 100 mg/kg | 0 | 100 | 120 | 140 | 160 |
| DS/Harm | | | | | |
| 150 mg/kg | 0 | 100 | 120 | 140 | 160 |
| DS/Harm | | | | | |

Veh = Vehicle,
Harm = Harmaline,
Prop = Propanolol,
DS = Drug Substance

TABLE H
Criteria for assessing subjective tremor score

| | Tremor | |
|---|---|---|
| SCORE | Level | Behaviour |
| 0 | Absent | Tremor absent |
| 1 | Mild | Occasional Tremor affecting only head and neck |
| 2 | Moderate | Intermittent tremor affecting all body |
| 3 | Moderate/ | Persistent tremor affecting all body and tail |
| | Severe | |
| 4 | Severe | Severe tremor rendering animal unable to stand |

### Drugs

Animals were dosed with vehicle, a positive comparator (propranolol), or test compound, and treated with harmaline or vehicle.

Drug substance (i.e., DS-D, as described in Example 1) was formulated in 1:12:87 Ethanol, Kolliphor HS & Saline.

Propranolol (40 mg/kg i.p.) served as a disease-relevant comparator.

30 mg/kg drug substance required 1 hr pre-treatment whilst 100 and 150 mg/kg drug substance required 2 hr pre-treatment.

### Statistical Analysis

Based on bioanalysis there was no suggestion that any animals should be removed. Behavioural observation data was analysed using a repeated measures two-way ANOVA followed by Dunnett's multiple comparison analysis (GraphPad, Prism v9.0.0) (p<0.05 considered significant). Data was not normally distributed, however, a QQ plot generated by ANOVA showed that it did not deviate from predicted line of normality and hence, an ANOVA was still applicable.

### Results

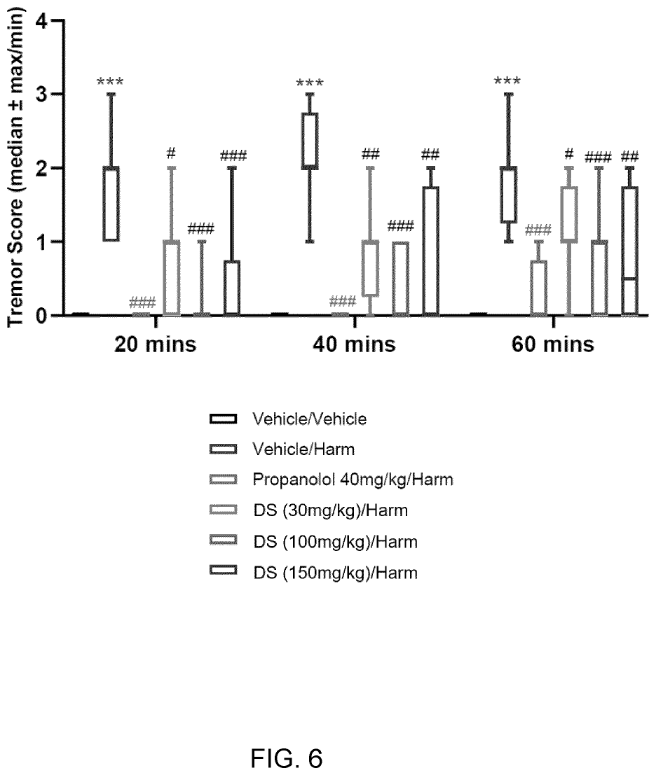

Data from this study is presented in FIG. 6 and Table I.

Harmaline at 10 mg/kg (5 mL/kg, i.p., T=0) induced a significantly higher tremor score at 20, 40, and 60 min post-dose (p<0.0001 when compared to the vehicle/vehicle group). This confirms that the model worked as expected for the indication of interest.

Propranolol at 40 mg/kg (5 mL/kg, i.p., T=−30 min) significantly attenuated the deficits induced by harmaline at 10 mg/kg at 20, 40, and 60 min post-harmaline when compared to the vehicle/harmaline group. This confirmed the expected result of a positive control.

Drug substance dosed at 30, 100 & 150 mg/kg i.p. significantly (p<0.05, p<0.01, p<0.001) reduced Harmaline tremor in rats at all timepoints tested, at 20, 40 and 60 mins post-harmaline administration. This is similar to the effect found for the positive control (propranolol) treatment groups.

TABLE I
Tremor Score

| | Tremor Score | | |
|---|---|---|---|
| | Timing post-harmaline (median ± IQR) | | |
| | 20 mins | 40 mins | 60 mins |
| Veh/Veh | 0.0 ± 0.0 | 0.0 ± 0.0 | 0.0 ± 0.0 |
| Veh/Harm | 2.0 ± 1.0 *** | 2.0 ± 0.75 *** | 2.0 ± 0.75 *** |
| Prop/Harm | 0.0 ± 0.0 ### | 0.0 ± 0.0 ### | 0.0 ± 0.75 ### |
| 30 mg/kg | 1.0 ± 1.0 # | 1.0 ± 0.75 ## | 1.0 ± 0.75 # |
| DS/Harm | | | |
| 100 mg/kg | 0.0 ± 0.0 ### | 0.0 ± 1.0 ### | 1.0 ± 1.0 ### |
| DS/Harm | | | |
| 150 mg/kg | 0.0 ± 0.0 ### | 0.0 ± 1.75 ## | 0.5 ± 1.75 ## |
| DS/Harm | | | |

# p < 0.05,
## p < 0.01,
### p < 0.001 veh/harmaline comparison or
*** p < 0.001 veh/veh comparison

### Conclusion

The present study showed that drug substance significantly reduced tremor in an animal model of essential tremor at all doses tested, 30, 100 and 150 mg/kg and across all time-points. Such a reduction was found to be in correspondence with the result of the positive control.

Thus, the applicant has surprisingly found that the complex botanical mixture of the present invention demonstrates significant efficacy in treating Essential Tremor in an in vivo model of Essential Tremor.

### Example 4: Effect of the Drug Substance on Essential Tremor

The following study was designed to assess the drug substance (DS) efficacy in the rat model of essential tremor. Synthetic CBD (sCBD I.P.) and DS I.P. and DS. P.O and compared observing tremor score, motion power percentage (MPP), tremor index (TI), number of tremors, total time spent in tremor, and average tremor length.

### Methods

Animals (254 male Sprague Dawley rats from Charles River UK, approximately aged 6-7 weeks, weight range 222-361 g) were housed in groups of 2-4, in standard caging with free access to food and water on a 12 h/12 h light/dark cycle from arrival (lights on at 7:00 AM) and were habituated to the tremor plate apparatus the day before the day of the experiment (2×5 min sessions 20 minutes apart). On the test day, the following treatments were administered:
At T=−100 min
   6, 12, 20 or 30 mg/kg Drug Substance (DS of Example 1) or vehicle via p.o.
   91.11 mg/kg sCBD via i.p.
   100 mg/kg Drug Substance (DS of Example 1) via i.p.
At T-=40 min:
   Vehicle only, 9.11 mg/kg, 27.33 mg/kg, or 54.67 mg/kg sCBD via i.p.
   Vehicle only, 10 mg/kg, 30 mg/kg, or 60 mg/kg Drub Substance (DS of Example 1) via i.p.
   Propranolol 20 mg/kg was given at T=−30 min and Harmaline was given at T=0 for all studies.

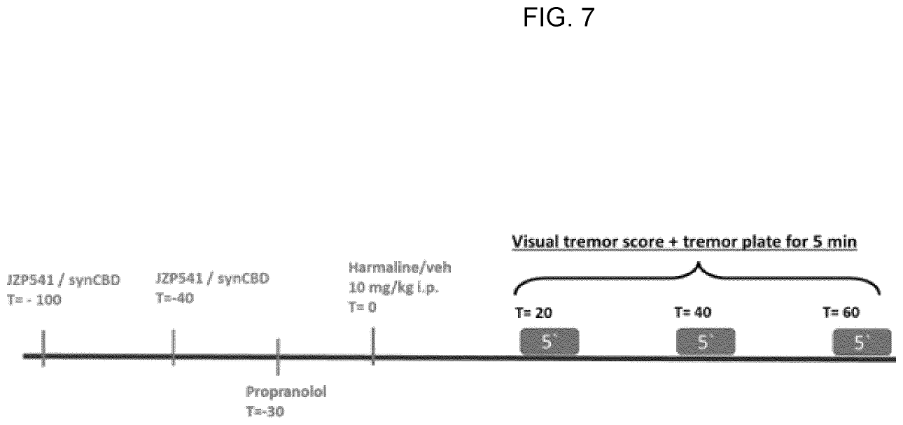

At T=20, 40 and 60 min, rats were observed for 30 sec in their home cage and then immediately placed on tremor plate for 5 min, as shown in FIG. 7.

### Results and Conclusions

The results are summarized in Tables J-M. All comparisons were made to the harmaline+vehicle group within each study.

TABLE J
Observed tremor score

| dose, | DS P.O. | | | DS I.P. | | | sCBD I.P. | | |
|---|---|---|---|---|---|---|---|---|---|
| mg/kg | 20 min | 40 min | 60 min | 20 min | 40 min | 60 min | 20 min | 40 min | 60 min |
| 4.8 | ns | ns | ns | NT | NT | NT | NT | NT | NT |
| 9.1 | * | ns | ns | ns | * | ns | ns | ns | ns |
| 16.0 | * | * | * | NT | NT | NT | NT | NT | NT |
| 27.3 | * | ** | ns | ** | **** | ** | ns | * | * |
| 54.7 | NT | NT | NT | ns | * | * | ** | * | * |
| 91.1 | NT | NT | NT | ** | *** | * | * | ns | ns |

TABLE K
Motion Power Percentage

| dose, | DS P.O. | | | DS I.P. | | | sCBD I.P. | | |
|---|---|---|---|---|---|---|---|---|---|
| mg/kg | 20 min | 40 min | 60 min | 20 min | 40 min | 60 min | 20 min | 40 min | 60 min |
| 4.8 | ns | ns | ns | NT | NT | NT | NT | NT | NT |
| 9.1 | ns | ns | * | ns | ns | ns | ns | ns | ns |
| 16.0 | ns | ns | ** | NT | NT | NT | NT | NT | NT |
| 27.3 | ** | ns | *** | * | ns | ns | ns | ns | ns |
| 54.7 | NT | NT | NT | * | ns | * | ns | ns | ns |
| 91.1 | NT | NT | NT | * | * | ** | ns | ns | * |

TABLE L
Tremor Index

| dose, | JZP541 P.O. | | | JZP541 I.P. | | | synCBD I.P. | | |
|---|---|---|---|---|---|---|---|---|---|
| mg/kg | 20 min | 40 min | 60 min | 20 min | 40 min | 60 min | 20 min | 40 min | 60 min |
| 4.8 | ns | ns | ns | NT | NT | NT | NT | NT | NT |
| 9.1 | * | ns | ns | ns | ns | ns | ns | ns | ns |
| 16.0 | * | ns | * | NT | NT | NT | NT | NT | NT |
| 27.3 | *** | * | ** | ** | ns | ns | ns | ns | ns |
| 54.7 | | | | *** | * | ** | ns | ns | ns |
| 91.1 | | | | **** | ** | ** | ns | ns | * |

TABLE M
Number of tremors, total time spent in tremor and average tremor length

| | JZP541 P.O. | | | JZP541 I.P. | | | synCBD I.P. | | |
|---|---|---|---|---|---|---|---|---|---|
| | Number | Total | Average | Number | Total | Average | Number | Total | Average |
| dose, | of | time in | tremor | of | time in | tremor | of | time in | tremor |
| mg/kg | tremors | tremor | length | tremors | tremor | length | tremors | tremor | length |
| 4.8 | ns | ns | ns | NT | NT | NT | NT | NT | NT |
| 9.1 | ns | ns | ns | ns | ns | ns | ns | ns | ns |
| 16.0 | ns | ns | ns | NT | NT | NT | NT | NT | NT |
| 27.3 | * | * | ns | ns | ns | ns | ns | ns | ns |
| 54.7 | NT | NT | NT | ns | ns | ns | ns | ns | ns |
| 91.1 | NT | NT | NT | ** | * | ** | ns | ns | ns |

ns = not significant; NT = not tested; *, **, ***, **** = statistical significance at p < 0.05, 0.01, 0.001, 0.0001 respectively, all comparisons made to harmaline + vehicle group.

The observed tremor score decreased following oral and intraperitoneal administration of DS comprising 9 mg/kg of CBD. Minimum sCBD dose of 27 mg/kg was necessary to achieve the same level of observed tremor score. Specifically, oral DS administration significantly reduced motion power percentage (MPP) and tremor index (TI) from the dose that comprised 9 mg/kg CBD for at least one time point. Intraperitoneal DS administration significantly reduced MPP and TI from the dose that delivered 27 mg/kg CBD. Synthetic CBD (sCBD) showed significant effect to reduce MPP and TI at 91 mg/kg only at one time point (60 min).

Significant negative correlation between plasma and brain concentrations and MPP was observed compared to the respective harmaline+vehicle control group, as summarized in Table N. Intraperitoneal administration of DS provided higher concentrations of THC, CBD and their metabolites in tissues and stronger correlations in MPP.

TABLE N
Correlation of motion power percentage (MPP) with
plasma and brain concentrations of CBD and THC

| Correlation | DS P.O. | DS I.P. | synCBD I.P. |
|---|---|---|---|
| vs MPP | Spearman r | Spearman r | Spearman r |
| Plasma CBD | −0.3589 * | −0.3589 * | −0.1554 ns |
| Brain CBD | −0.3899 ** | −0.4472 ** | −0.2171 ns |
| Plasma 6-OH-CBD | −0.3114 * | −0.5685 **** | −0.2517 ns |
| Brain 6-OH-CBD | −0.3150 * | −0.5806 **** | −0.2533 ns |
| Plasma 7-OH-CBD | −0.3102 * | −0.4956 *** | −0.2539 ns |
| Brain 7-OH-CBD | Brain | 0.1233 ns | −0.3445 ns |
| | concentrations | | |
| | too low | | |
| Plasma 7-COOH-CBD | −0.2924 * | −0.4923 *** | −0.2413 ns |
| Brain 7-COOH-CBD | Brain | Brain | −0.3006 ns |
| | concentrations | concentrations | |
| | too low | too low | |
| Correlation | JZP541 P.O. | JZP541 I.P. | synCBD I.P. |
| vs MPP | Spearman r | Spearman r | Spearman r |
| Plasma THC | −0.4023 ** | −0.4223 ** | NA |
| Brain THC | −0.3854 ** | −0.4575 ** | NA |
| Plasma 11-OH-THC | −0.2861 ns | −0.2647 ns | NA |
| Brain 11-OH-THC | −0.2488 ns | −0.4343 ** | NA |

### Example 5: Effect of Drug Substance on Behavioural Measures with Relevance to Autism Spectrum Disorder

Prenatal administration of valproic acid (VPA) has been used extensively as a model of ASD. The VPA model in rodents has significant effects on rodent-equivalent measures of core behavioural characteristics of ASD in humans, including social impairments, repetitive behaviour, cognitive abilities, and non-core characteristics including irritability, aggression, and hyperactivity.

In the present study, the effect of the drug substance on core symptoms and non-core symptoms, in particular irritability, was investigated. Specifically relevant to stereotyped, repetitive behaviours, the following behavioural aspects were measured: self-grooming behaviour and burying objects using the open field test. Relevant to social interaction deficits, social preference using social avoidance approach task was measured. Relevant to non-core, associated symptoms, locomotor activity was measured using the open field task and irritability was measured using the bottlebrush test in adults. Additionally, social play behaviour and irritability were assessed in juvenile animals following sub-chronic drug administration. Sub-chronic risperidone was used as a positive control as it has been associated with significant reduction in increased grooming behaviour and hyperactivity associated with the VPA model.

### Methods

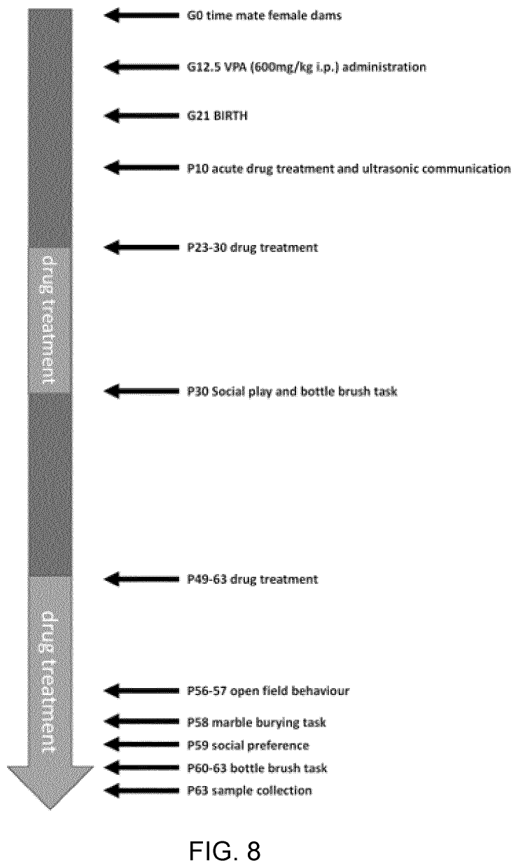

Time-mated female Wistar rats were administered a single dose of valproic acid (VPA, 600 mg/kg, i.p.) on gestational day 12.5. Male offspring were delivered and confirmation of the VPA induced developmental deficit was rapidly achieved by examination of characteristic “tail kinks” in the juvenile animals. The timeline of experiments is outlined in FIG. 8 and animals were randomly allocated to treatment groups (Table O).

Animals were administered drug substance (DS-E, exemplary specification provided in Example 1), vehicle or risperidone (positive control) once daily for 8 days and social play and bottle brush irritability tests were performed on day 8 (P30). Control animals were administered sterile saline (0.9% w/v, i.p.) in utero and the vehicle control (ethanol: Kolliphor HS15: saline (1:1:18, v/v/v), i.p.

TABLE O
Sample size per group, doses and pre-treatment times.

| | Prenatal | Postnatal | Pre-treatment | Sample |
|---|---|---|---|---|
| Groups | treatment | treatment | time | size |
| Group A | In utero | Vehicle | 60 mins | 8 |
| | vehicle | | | |
| Group B | In utero VPA | Vehicle | 60 mins | 8 |
| Group C | In utero VPA | Drug substance | 60 mins | 8 |
| | | (10 mg/kg) | | |
| Group D | In utero VPA | Drug substance | 60 mins | 8 |
| | | (30 mg/kg) | | |
| Group E | In utero VPA | Drug substance | 120 mins | 8 |
| | | (100 mg/kg) | | |
| Group F | In utero VPA | Risperidone | 60 mins | 8 |
| | | (0.125 mg/kg) | | |

| | |
|---|---|
| Total | 48 |

### Detailed Methods

### Direct Social Interaction and Social Play-P30

At postnatal day 30 and following 8 days treatment with the test compounds, animals were evaluated for juvenile play behavior. This test was carried out under low light in a novel test arena (40×25 cm), wherein paired animals from the same treatment group are examined over a 15-minute trial. Animals were first allowed to individually explore the arena for 10-minute acclimatization trial (P29) and 24 hours later evaluated for social play (P30). On day of evaluation animals were scored for play activity measured by pinning (latency to, total duration and frequency), mounting (latency to, total duration and frequency) and latency, frequency and duration of social play behavior (following or approaching test partner, sniffing or social grooming). Scoring was conducted blind by manual video analysis employing Ethovision XT (Noldus, UK) software linked to an overhead video camera.

### Open Field Test (Locomotor Activity, Circling Behaviour, Anxiety, Grooming Behaviour, Stereotypy) P56-57

Animals were scored for locomotor activity and exploratory behavior in a two 10-minute trials within an open field arena over two days. Each day the animals were placed into a 64 cm2 black Perspex box with 30 cm side walls, under low light and monitored via remote video capture for spontaneous activity. Linked to Ethovision XT image analysis software this was analysed for total distance moved and number and duration of center zone crossings. Manual scoring of the videos was then conducted (blinded to treatment group) to determine frequency of circling behavior (number of 360 degree turns) and time spent grooming.

### Marble Burying Test P58

The marble burying test has been employed as an animal model of anxiety and impulsive behavior. We have adapted the model from the standard mouse variant whereby the test animal is placed in a cage measuring 45×25×25 cm (l×w×h), containing 9 2.5 cm diameter marbles distributed evenly and sitting on top of 5 cm deep sawdust substrate (Schneider and Popik, 2007 Psychoneuroendocrinology 32:651). Over a 10-minute test period animals were allowed to freely explore the apparatus and subsequently removed whereupon marbles covered by at least one half with bedding were considered buried and this number was recorded and was the main outcome measure for this behavior. Video recording of the test period was evaluated for locomotor activity and scored for frequency and duration of digging behavior.

### Irritability-Like Behaviour: Bottle Brush Test P30 and P61-64

To test irritability-like behavior we employed the bottle-brush test modified for rats (Riittinen et al. 1986 Dev Psychobiol 19:105-111). Irritability-like behavior was examined by measuring aggressive and defensive responses during the bottle-brush test carried out in juvenile animals immediately after play behavior evaluation at P30 and again in adult animals on P59. Irritability-like behavior testing was performed under dark conditions with a red light and infrared camera recording. Testing consisted of 10 trials per rat in plastic cages (377×325×170 mm) with fresh bedding. During each trial, the rat was placed at the back of the cage. A bottle-brush was rotated toward the animal's whiskers (from the front of the cage). The brush was rotated around the whiskers of the rat for approximately 1 second. The brush was then rotated back to the front of the cage where it hangs vertically for approximately 2 seconds, during which behavioural responses were recorded. An intertrial interval of 10 second was used.

Employing video analysis of the test, the following were scored as aggressive responses: smelling the brush, biting the brush (during the initial phase of rotating the brush forward and back to the starting position), boxing the target, following the brush, exploring the brush (using paws or mouth to manipulate the brush without biting or boxing), mounting the brush, and delayed biting (during the 2 s that the brush hung at the starting position). Additionally, the following were scored as defensive responses: escaping from the brush, digging, burying, jumping, climbing, vocalization, freezing, and grooming. Frequency of each response and the sum of aggressive and defensive behaviours were recorded.

### Social Interaction: Social Approach Avoidance (SAA) P60

This test was originally used for assessing anxiety-like behavior and subsequently used to assess the social behavior in potential animal models of schizophrenia (Sams-Dodd, 1995 Behav Pharmacol. 6:55) but has been extended to the study of autism models (Schneider and Przewlocki, 2005 Neuropsychopharmacol. 30:80). Therefore, utilising a model based on published designs (Brodkin et al., 2004 Brain Res. 1002:151; Moy et al., 2004 Genes Brain Behav. 3:287) adult animals were assessed for social preference following prenatal VPA exposure whereby a novel animal was introduced to the test arena (n=8). The main outcome measure was the time engaged in active social behavior. Additionally, the time spent in each of the social, non-social and neutral chamber was also determined.

### Statistical Methods

All data was assessed for outliers (ROUT; Prism V9.02) and then normality using D'Agostino and Pearson normality test for n=8, or Shapiro-Wilks test for n<8 or unequal groups. Data that were non-normally distributed were analysed using Kruskal-Wallis followed by planned comparisons with Dunn's correction. Normally distributed data were analysed using one-way ANOVA or two-way ANOVA followed by planned comparisons with Sidak's correction. Data that follow a normal distribution are presented as mean±SEM, data that do not follow a normal distribution are presented as box and whisker plots with individual values plotted (median and IQR, with the tails showing the highest and lowest values). Statistical significance was defined as p<0.05 using Prism 9.0.2 software for analysis.

### Results

### Juvenile-Irritability

Overall, in juvenile animals, there was significant increase in irritability in VPA-treated group compared to vehicle treated animals. Sub-chronic administration of the positive control risperidone had no effect on increased irritability behaviour shown in the rat VPA model. Sub-chronic administration of drug substance at the highest dose (100 mg/kg) as compared to vehicle was associated with significant reduction in irritability score in the rat VPA model.

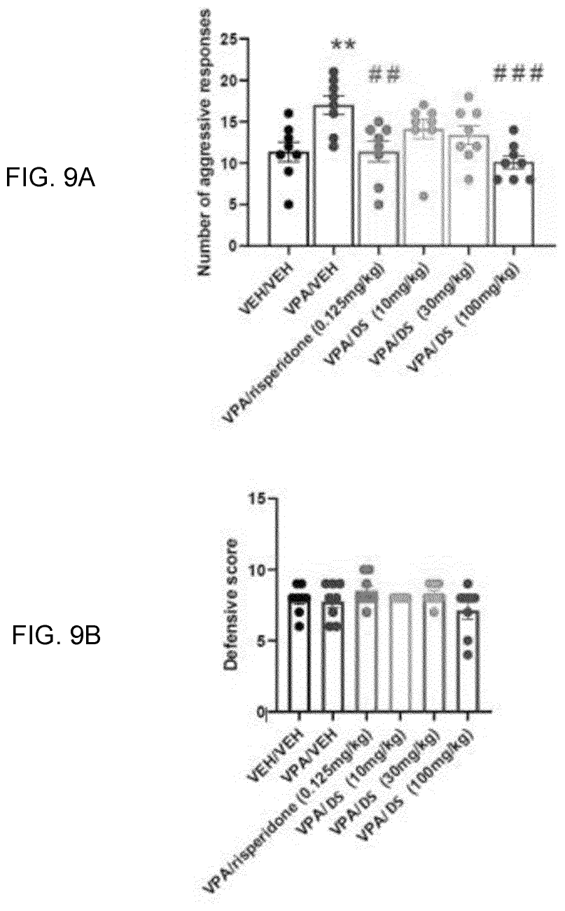

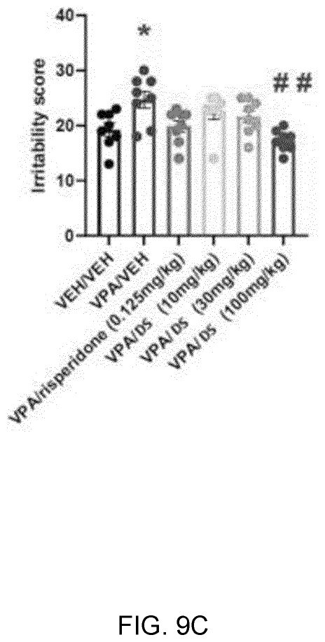

With regard to aggressive responses, there was a significant main effect of treatment on aggressive responses (FIG. 9A; P=0.0002). Further investigation revealed significant increases in aggressive responses in VPA treated rats compared to vehicle control (VEH/VEH vs VPA/VEH, P=0.002). Drug substance at 100 mg/kg relative to vehicle significantly reduced the increase in aggressive responses seen in the rat VPA model (FIG. 9A, P=0.0002 100 mg/kg drug substance vs VPA/VEH). Risperidone also significantly reduced the increase in aggressive responses seen in the VPA model (FIG. 9A, P=0.002 risperidone vs VPA/VEH).

With regard to defensive responses, there was no significant main effect of treatment on defensive responses (FIG. 9B, P=0.24).

With regard to irritability, there was a significant main effect of treatment on irritability (combined aggressive and defensive behaviours) (FIG. 9C, P=0.0024). Further investigation revealed significant increases in irritability score in VPA treated rats compared to vehicle control (VEH/VEH vs VPA/VEH, P=0.044). Drug substance at 100 mg/kg relative to vehicle significantly reduced the increase in aggressive responses seen in the rat VPA model (FIG. 9C, P=0.001 vs VPA/VEH). Risperidone has no effect in irritability score seen in the VPA model (FIG. 9A, P=0.10 risperidone vs VPA/VEH).

### Juvenile-Social Play

Overall, there were no significant treatment changes in latency, frequency or duration in mounting nor pinning behaviour. There was significant reduction in social duration and frequency behaviours and no change in social latency in VPA-treated group compared to vehicle treated animals. Social duration and frequency behaviours were significantly increased by the sub-chronic administration of 30 mg/kg and 100 mg/kg drug substance. Risperidone had no effect on these behaviours.

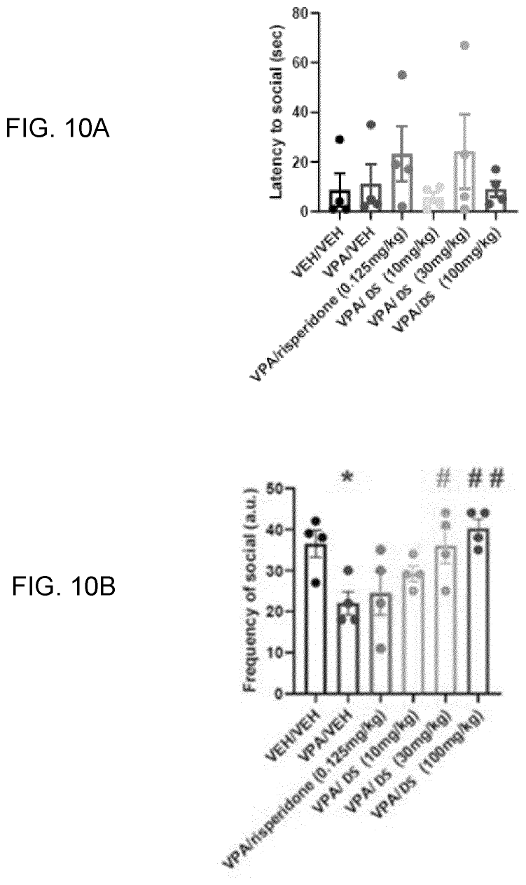

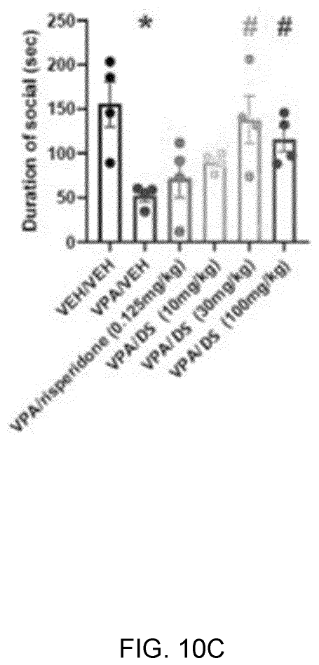

When investigating reciprocal social behaviour, one-way ANOVAs revealed no significant treatment effects on latency to play (P=0.56) (FIG. 10A); however there were significant main effects of treatment on both frequency and duration of social play (frequency of social play: P=0.0085 and duration of social play: P=0.0076) (FIGS. 10B-C). Further investigation revealed significant effects of valproic acid exposure when compared to vehicle treated control animals (VEH/VEH vs VPA/VEH, P=0.034); as well as significant effects of both 30 and 100 mg/kg drug substance treatment compared to vehicle treated VPA animals (P=0.042 and P=0.0068). Pairwise analysis of the duration of social play showed significant effects of valproic acid compared to control animals (VEH/VEH vs VPA/VEH, P=0.0042) and 30 mg/kg drug substance treated animals (P=0.019) when compared to vehicle VPA animals.

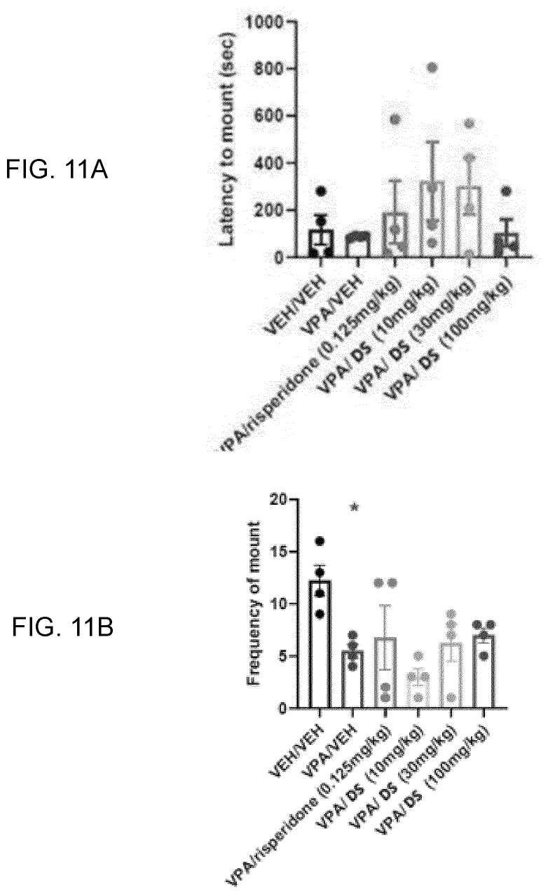

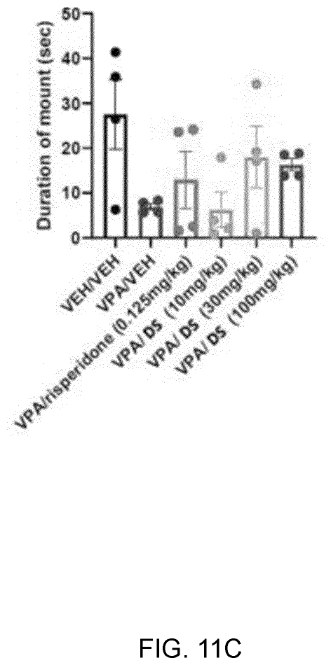

When investigating mounting behaviours, one-way ANOVAs showed there was no significant main effects of treatment on either latency or duration measures (P=0.47; and P=0.09) (FIGS. 11A and 11C). A one-way ANOVA did reveal a significant main effect of treatment on the frequency of mounting play (P=0.02) (FIG. 11B). Post-hoc pairwise analysis showed a significant effect of valproic acid exposure when compared to vehicle controls (VEH/VEH vs VPA/VEH, P=0.40).

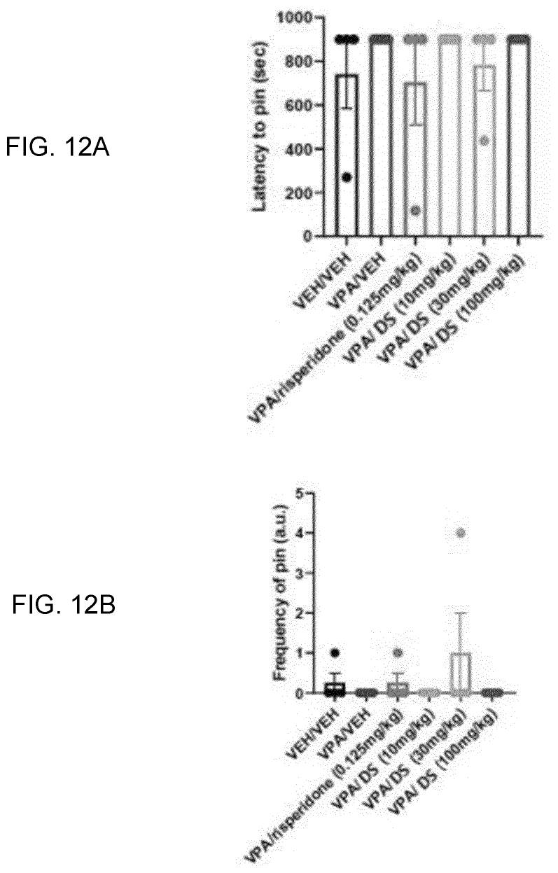

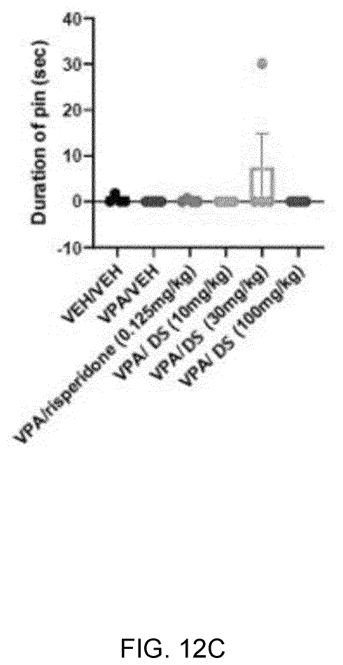

There were no significant main effects of treatment on the frequency, latency, or duration of pinning observed (P=0.54; 0.63, P=0.68; and P=0.46) (FIGS. 12A-C). Risperidone (0.125 mg/kg) was without effect on any measure of play or social interaction in juvenile animals.

### Adult-Irritability

Irritability was measured using the bottlebrush test and was measured for a total of four consecutive days. Irritability score was significantly higher in VPA treated rats as compared to vehicle treated controls. Sub-chronic administration of risperidone significantly reduced irritability score in VPA-treated rats. Sub-chronic drug substance administration at 30 mg/kg and 100 mg/kg doses significantly reduced irritability score on days 1˜4 of assessment and at 10 mg/kg dose on day 4 of assessment.

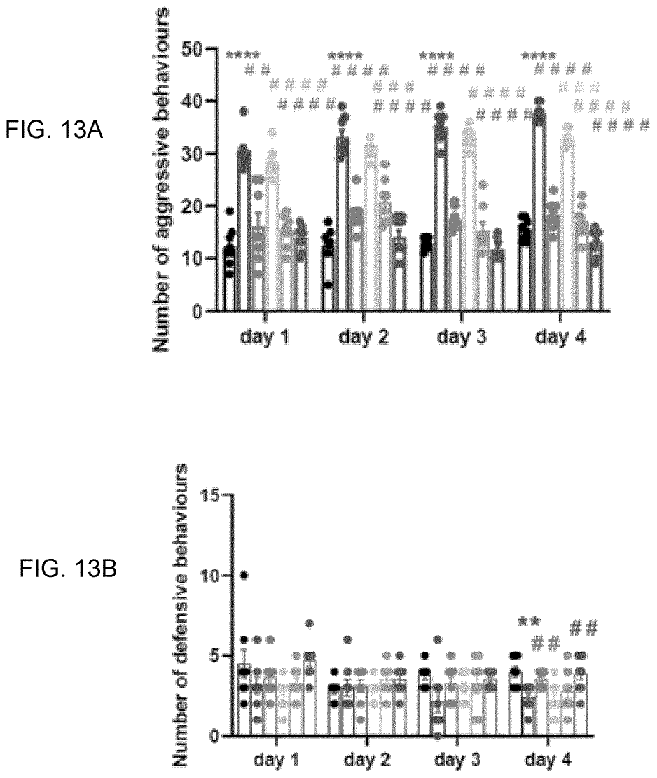

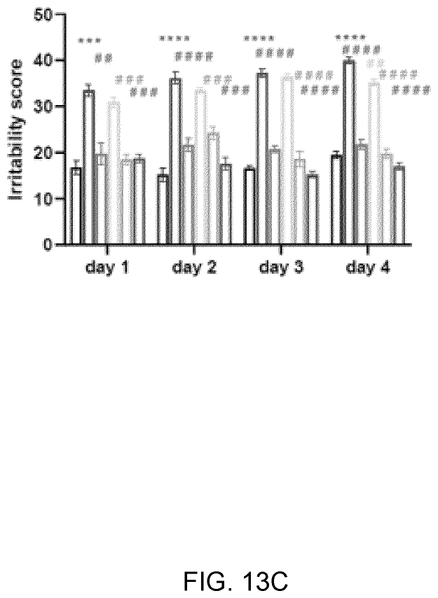

With regard to aggressive responses, two-way ANOVA showed there was a significant effect of treatment (p<0.0001), there was a significant effect of day (p<0.0001) and significant interaction (P=0.0005). Specifically, there was a significant increase in aggressive behaviour in VPA treated group compared to vehicle across all days of assessment (P<0.0001). Moreover, drug substance at 30 mg/kg and 100 mg/kg significantly reduced the increase in aggressive behaviour induced by VPA (P<0.0001) on all days of assessment, whereas 10 mg/kg drug substance significantly reduced the increase in aggressive behaviour on day 4 of assessment only (P<0.001). Risperidone significantly reduced the increase in aggressive behaviour across all days of assessment (Day 1 P<0.01; P<0.0001 days 2-4) (FIG. 13A).

With regard to defensive responses, two-way ANOVA showed there was a significant effect of treatment (p=0.0002), no significant effect of day (P=0.13) and no significant interaction (P=0.36). There was a significant reduction in defensive behaviours in VPA model compared to vehicle control (P=0.013), an effect that was significantly increased by drug substance at 100 mg/kg (P=0.0009), but no significant effect of risperidone (P=0.09) (FIG. 13B).

With regard to irritability, two-way ANOVA showed there was a significant effect of treatment (P<0.0001), there was a significant effect of day (P=0.02) and significant interaction (P=0.0004). There was significant increase in irritability score in the rat VPA model as compared to vehicle control at all days of assessment (Day 1=0.0004, Day2-4=P<0.0001). Moreover, drug substance significantly reduced the increase in irritability score at 30 and 100 mg/kg relative to the VPA model throughout all days of assessment (For 30 mg/kg: Day 1 P=0.0005, Day 2 P=0.0007, Day 3&4 P<0.0001; For 100 mg/kg: Day 1 P=0.0004, Day 2 P=0.0001, Day 3&4 P<0.0001) and at 10 mg/kg significantly reduced the increase in irritability score on day 4 (P<0.004). The positive control risperidone significantly reduced the increase in irritability score throughout all four assessment days (Day 1=0.0044, Day2-4=P<0.0001) (FIG. 13C).

### Adult-Social Interaction

Social interaction was assessed using the social approach avoidance task. The time engaged in active social behaviour with a novel animal was significantly reduced in the VPA treated group compared to control group. Moreover, sub-chronic administration of drug substance at 30 mg/kg and 100 mg/kg was associated with significant increase in time engaged in active social behaviour as compared to vehicle-treated VPA rats. Sub-chronic administration of risperidone had no effect on this behaviour.

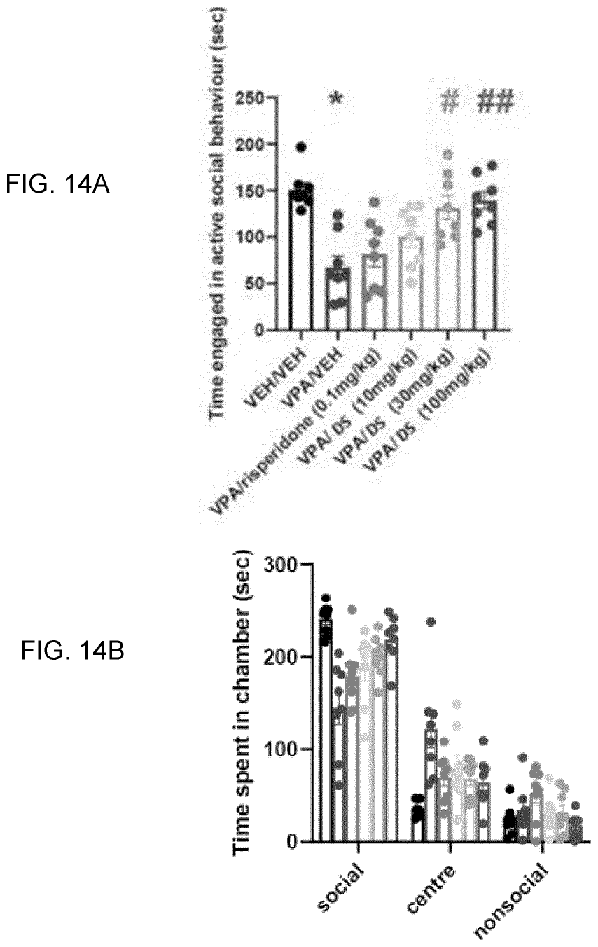

There was a significant main effect of treatment on the time engaged in active social behaviour (FIG. 14A, P=0.0002). Specifically, there was a significant reduction in the time engaged in active social behaviour in the VPA model as compared to healthy controls (P=0.0003), a behavioural deficit that was significantly prevented by the administration of drug substance at 30 (P=0.038) and 100 mg/kg (P=0.0054), but not prevented by risperidone.

Additionally, the time spent in the chambers was also investigated to rule out this confounding effect on the time engaged in active social behaviour. There was no significant effect of treatment on the time spent in the chambers (FIG. 14B, P>0.05); however, there was a significant effect of chamber (P<0.0001) and significant interaction between chamber and treatment (P<0.0001). Therefore, the time spent in each of the chambers did not have an impact on the effects on active social behaviour. Therefore, the observed improvement in social interaction is related to both a preference for social contact and increased active social behaviour for the effective doses of drug substance and not a change in exploratory behaviour or sedation.

### Adult-Circling and Grooming Behaviour

Circling behaviour was significantly increased in the rat VPA model compared to vehicle control. Risperidone (0.125 mg/kg) and drug substance at 10 and 100 mg/kg significantly reduced circling behaviour on day 2.

There was a significant increase in grooming behaviour in VPA treated group compared to vehicle treated group. Moreover, sub-chronic risperidone (0.125 mg/kg) and sub-chronic drug substance treatment at 30 mg/kg and 100 mg/kg significantly reduced the increase in grooming behaviour in VPA treated rats.

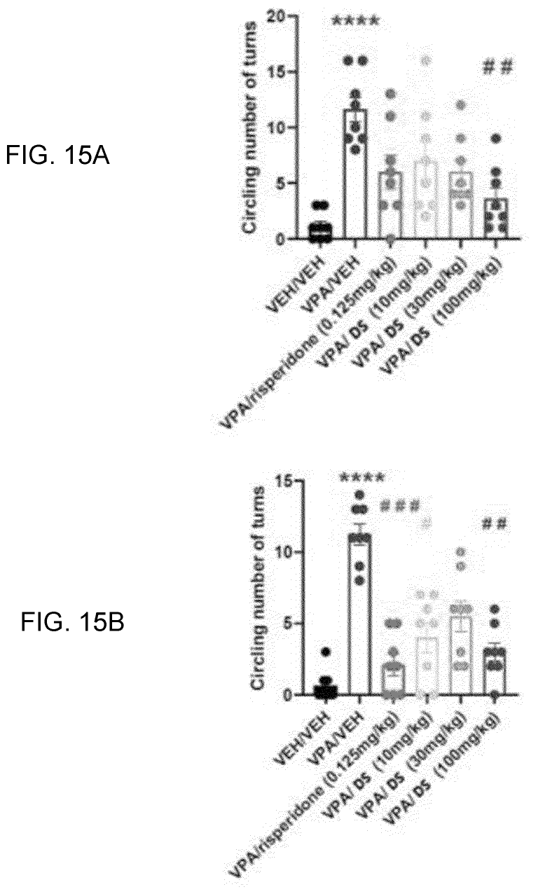

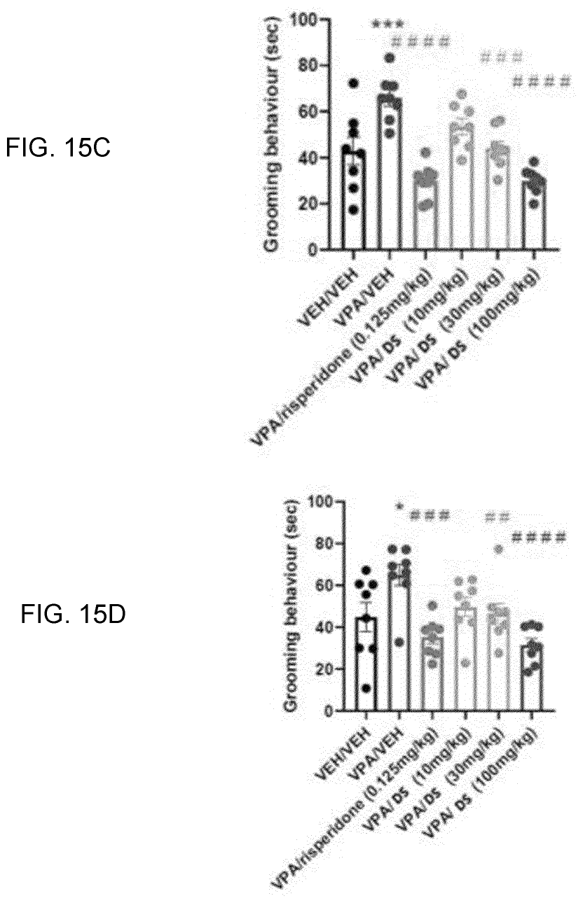

On day 1 there was a significant main effect of treatment on circling behaviour (P=0.0001) (FIGS. 15A-D). Specifically, there was a significant increase in circling behaviour in the rat VPA model relative to control (P<0.0001), that was significantly reversed by drug substance 100 mg/kg group (P=0.005). Additionally, on day 1 there was a significant main effect of treatment on grooming behaviour (P<0.0001) (FIG. 15C). Specifically, there was a significant increase in grooming in the VPA model as compared to the vehicle control (P=0.0003). Additionally, risperidone (P<0.0001) as well as drug substance at 30 (P=0.0006) and 100 mg/kg (P<0.0001) doses significantly reduced the increase in grooming behaviour in the VPA model.

On day 2, there was a significant main effect of treatment on circling behaviour (P=0.00001) (FIG. 15B). Specifically, there was a significant increase in circling behaviour in the VPA model as compared to vehicle control (P=0.0004). Moreover, risperidone (P=0.0004) and drug substance at 10 (P=0.0238) and at 100 mg/kg (P=0.0062) significantly reversed the increase in circling behaviour. On day 2, there was a significant main effect of treatment on grooming behaviour (P=0.0003) (FIG. 15D). Specifically, there was a significant increase in grooming in the VPA model as compared to the vehicle control (P=0.023). Additionally, risperidone (P=0.0004) as well as drug substance at 30 (P=0.0415) and 100 mg/kg (P<0.0001) doses significantly reduced the increase in grooming behaviour in the VPA model.

### Adult-Marble Burying

The primary outcome measure was the number of marbles buried and that was significantly increased in the VPA model compared to control. Sub-chronic risperidone (0.125 mg/kg) as well as sub-chronic drug substance at all three doses tested (10, 30 and 100 mg/kg) significantly reduced the number of marbles buried.

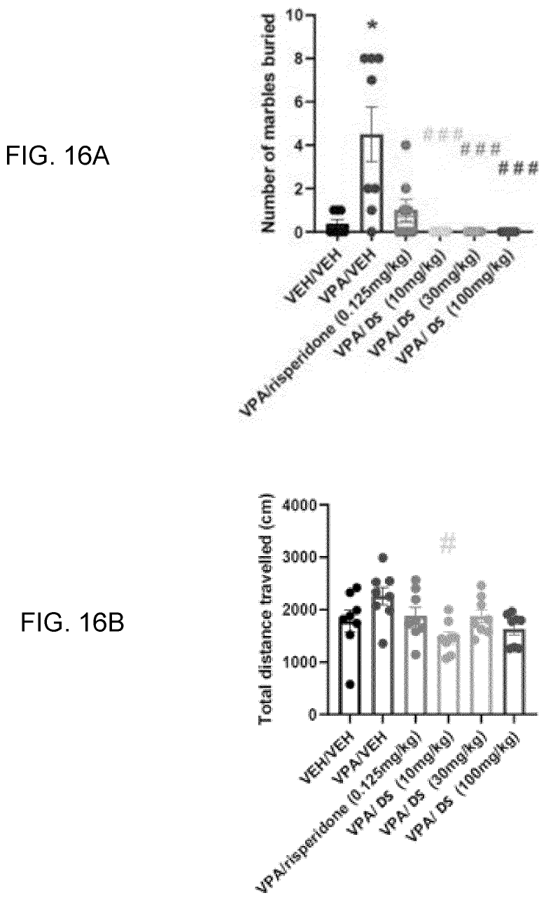

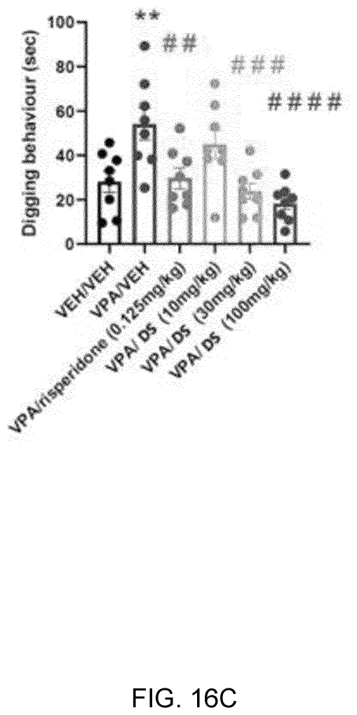

Burying the novel objects in substrate indicates an aversive response towards them and may also indicate irritability as a result of VPA exposure. There was a significant effect in the number of marbles buried between treatment groups (P=0.0009) (FIG. 16A). Specifically, there was a significant increase in the number of marbles buried in the VPA model as compared to vehicle controls (P=0.025). Additionally, drug substance at all three doses tested significantly reduced the VPA induced increase in marble burying behaviour (10, 30 and 100 mg/kg drug substance vs VPA/Veh P=0.0013). Risperidone had no significant effect on VPA induced increase in the number of marbles buried.

While there was a significant effect of treatment on distance travelled (P=0.029), there was no significant change between VPA model and vehicle control (FIG. 16B).

There was a significant effect of treatment on digging behaviour (FIG. 16C, P<0.0001). Specifically, there was a significant increase in digging behaviour in the VPA model when compared to vehicle control (P=0.0043). Moreover, drug substance at 30 (P=0.0007) and 100 mg/kg (P<0.0001), but not at 10 mg/kg (P=0.61), significantly reduced the increase in VPA-induced digging behaviour. Additionally, risperidone significantly reduced the increase in VPA-induced digging behaviour (P=0.008).

### Adult-Locomotor Activity

Spontaneous locomotor activity as measured by total distance travelled was significantly increased in rat VPA model as compared to vehicle controls on days 1 and 2 of testing. Risperidone as well as drug substance at 30 and 100 mg/kg administration significantly reduced the increase in spontaneous locomotor activity on day 1. On day 2, locomotor activity was significantly increased in rat VPA model as compared to vehicle controls. Risperidone and drug substance at the highest dose (100 mg/kg) significantly reduced locomotor activity on day 2 of testing.

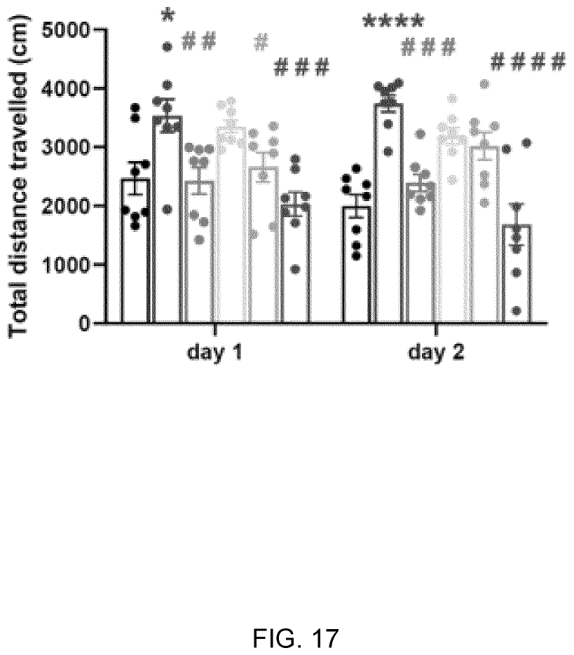

All groups of animals were evaluated in the open field over two days following 8 and 9 days treatment with the drug substance (10, 30 and 100 mg/kg), vehicle or risperidone. On day one, there was a significant mean effect of treatment on distance travelled (FIG. 17; P=0.0002). Specifically, there was a significant increase in locomotor activity in VPA-treated animals as compared to vehicle controls (P=0.010 VPA/VEH vs VEH/VEH). Drug substance at 30 mg/kg and 100 mg/kg, as well as risperidone, significantly prevented the increase in locomotor activity in VPA rats (P=0.044 VPA/Veh vs 30 mg/kg drug substance; P=0.0002 VPA/Veh vs 100 mg/kg drug substance and P=0.007 VPA/Veh vs risperidone).

On day two, distance travelled was also significantly altered (FIG. 17; P<0.0001). Specifically, locomotor activity was significantly increased in VPA exposed animals as compared to vehicle controls (P<0.0001 VPA/VEH vs VEH/VEH). Drug substance at 100 mg/kg, as well as risperidone, significantly prevented the increase in locomotor activity in VPA rats (P<0.0001 VPA/Veh vs 100 mg/kg drug substance and P=0.0003 VPA/Veh vs risperidone).

### Activity in the Centre Zone of the Arena

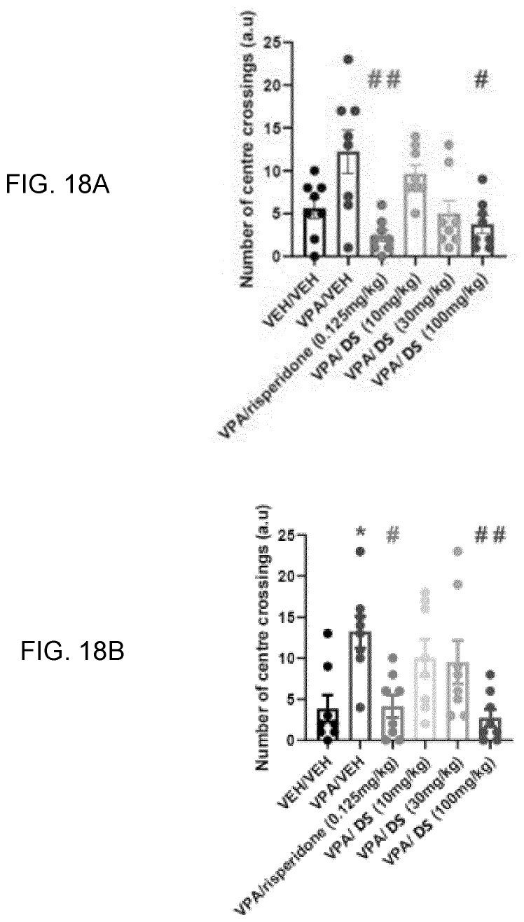

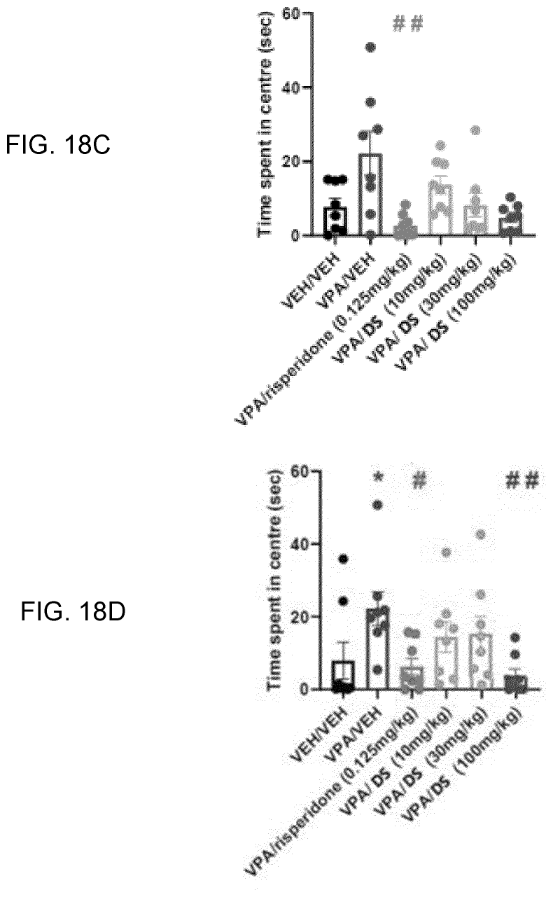

On day 1, there was a significant effect of treatment in the frequency of centre zone crossings (FIG. 18A, P=0.0017). Specifically, there was no significant difference in the frequency of centre zone crossings by VPA exposed animals compared to vehicle controls on day one (P>0.05). There was a significant decrease in the frequency of centre zone crossings in drug substance 100 mg/kg (P=0.04) group compared to VPA/Veh group and in risperidone group as compared to VPA/Veh group (P=0.0033). Moreover, there was a significant effect of treatment in time spent in centre zone (FIG. 18C, P=0.0068). Specifically, there was no significant difference in the time spent in the centre zone in VPA exposed animals compared to vehicle controls on day one (P=0.38). On day 1, drug substance had no effect on time spent in the centre zone (P=0.065). Risperidone (0.125 mg/kg) significantly reduced the time spent in the centre zone on day one (P=0.004).

On day 2, there was a significant effect of treatment in the number of centre zone crossings (FIG. 18B, P=0.0017). Specifically, there was a significant increase in the number of centre zone crossings in VPA model compared to vehicle controls (P=0.013). Moreover, drug substance at 100 mg/kg (P=0.003) and risperidone (P=0.0221) significantly reduced the VPA induced increase in the number of centre zone crossings. On day 2, there was a significant effect of treatment in the time spent in the centre zone (FIG. 18D, P=0.0035). Specifically, there was a significant increase in the time spent in the centre zone by the VPA treated animals as compared to vehicle controls (P=0.0179). Drug substance at 100 mg/kg on day two, significantly reduced the time spent in the centre zone as compared to VPA/VEH group (FIG. 18D, P=0.0037). The reference compound risperidone (0.125 mg/kg) significantly reduced the VPA induced increase in the total time spent in the centre zone (P=0.0297).

### Conclusions

This study has demonstrated deficits in social duration and frequency behaviours during social play test and increase in irritability behaviour in the juvenile rat VPA model. Sub-chronic administration of risperidone did not have a significant effect on these behaviours, suggesting a proportion of the models being refractory to risperidone treatment. Sub-chronic administration of drug substance significantly increased social duration and frequency behaviours at 30 mg/kg and 100 mg/kg drug substance. Moreover, sub-chronic drug substance administration at 30 mg/kg and 100 mg/kg was associated with significantly reduced irritability score on days 1˜4 of assessment and at 10 mg/kg dose on day 4 of assessment.

This study has demonstrated deficits in core symptoms specifically deficits in social interaction, increased grooming behaviour and marble burying as well as hyperlocomotion and circling behaviour in adult rat VPA model compared to control. Additionally, irritability behaviour was also significantly increased in adult rat VPA model. Sub-chronic risperidone used as the positive control in this study, significantly reduced hyperlocomotion, reduced the increase in marble burying, grooming behaviour and irritability but did not have a significant effect on social interaction. Sub-chronic drug substance administration was associated with significant reduction in hyperlocomotion, reduction in increase in marble burying, grooming behaviour and irritability and improved social interaction. Drug substance treatment (10, 30, and 100 mg/kg, IP) dose-dependently reversed irritability, deficits in social interaction, repetitive-like behaviour, and hyperactivity in the adult model. Moreover, drug substance (30-100 mg/kg, IP) was superior to the positive control risperidone (0.125 mg/kg, IP) in some of the behavioural domains assessed, particularly in reversing the deficits in social behaviour, where risperidone was shown to be inactive.

Thus, the in vivo efficacy pharmacology study demonstrated that the drug substance of the present invention significantly reversed VPA-induced behavioural deficits in the model with relevance to irritability and the core symptoms of ASD. Drug substance significantly reduced irritability behaviour (P<0.01-0.0001), hyperactivity (P<0.01-0.0001), stereotypy (P<0.05-0.0001) and increased active social behaviour (P<0.05-0.01). Deficits in social, stereotypical, exploratory and irritability behaviour are reduced by treatment with the drug substance for all measures tested in a dose dependent manner.

### Example 6: Effect of Drug Substance on Autism Spectrum Disorder (ASD)-Like Behaviors

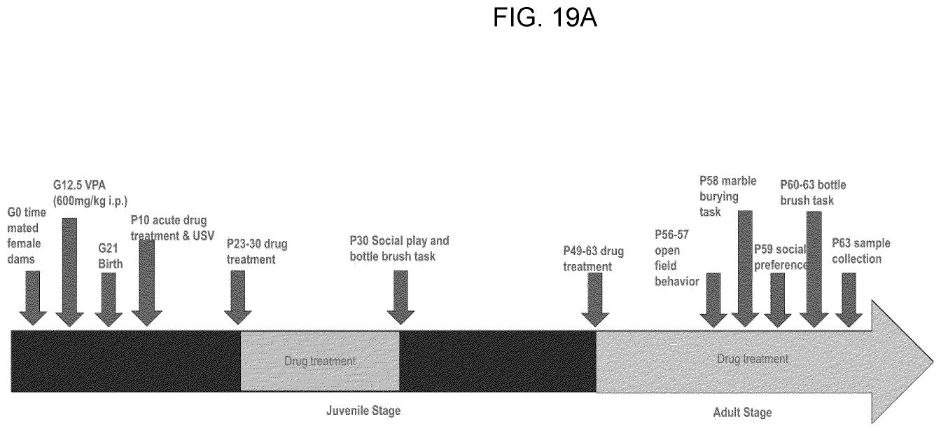

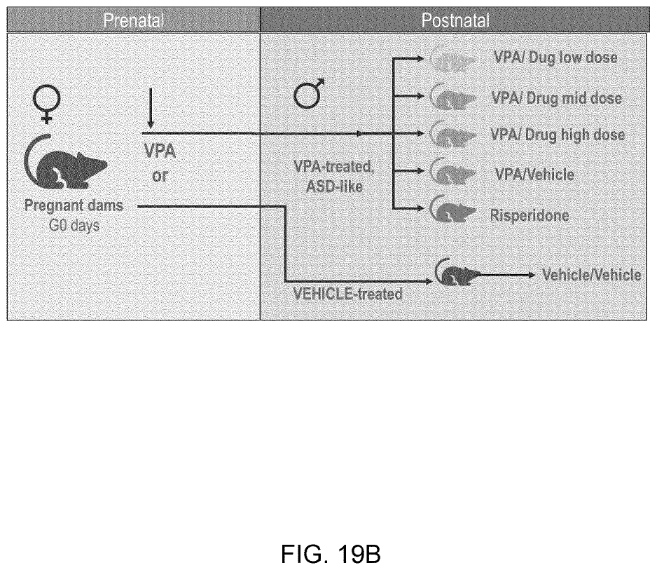

The main objective of the following study was to assess the effect of the drug substance (DS) of Example 1 in ameliorating autism-like behavior in valproic acid-induced autism spectrum disorder (ASD) mouse model (FIGS. 19A-B). The therapeutic effects of DS (administered via i.p.) were compared with the effects of risperidone and synthetic CBDs (sCBD). The results are summarized in Table P. A different dose response pattern of DS in ameliorating autism-like behaviors was observed compared to sCBD. The DS provided a minimum effective dose of 30 mg/kg (i.p.) in social behavior and after the bottle-brush test (irritability) whereas sCBD minimum effective dose was 92.11 mg/kg (i.p.).

TABLE P
Comparison of DS and sCBD1 in the rat VPA model of ASD-like behaviors

| | | | | Risperidone | | | | Risperidone | | | | Risperidone |
|---|---|---|---|---|---|---|---|---|---|---|---|---|
| | DS, mg/kg | | | 0.125 | | | | 0.125 | sCBD-2, mg/kg | | | 0.125 |
| | I.P. | | | mg/kg | sCBD, mg/kg I.P. | | | mg/kg | P.O. | | | mg/kg |
| Readout | 10 | 30 | 100 | I.P. | 9.21 | 27.63 | 92.11 | I.P. | 10 | 30 | 100 | I.P. |
| Juvenile vocalisation | − | − | + | − | − | − | + | − | − | − | + | − |
| Juvenile play behavior | Ø | + | + | Ø | Ø | Ø | + | + | Ø | + | + | Ø |
| Juvenile bottle brush irritability test | Ø | Ø | + | Ø | Ø | + | + | + | Ø | Ø | + | + |
| Adult open field hyperactivity | Ø | + | + | + | + | + | + | + | Ø | Ø | + | + |
| (spontaneous LMA on day 1) | | | | | | | | | | | | |
| Adult open field hyperactivity (LMA on | Ø | Ø | + | + | Ø | + | + | + | + | + | Ø | Ø |
| day 2) | | | | | | | | | | | | |
| Adult marble burying | + | + | + | + | Ø | + | + | + | Ø | + | + | + |
| Adult grooming | Ø | + | + | + | + | + | + | + | No phenotype | | | |
| Adult active social behavior | Ø | + | + | Ø | Ø | Ø | + | + | Ø | Ø | + | Ø |
| Adult bottle brush irritability test | Ø | + | + | + | Ø | Ø | + | + | Ø | + | + | + |

+ improvement,
Ø no change,
− not tested

### Example 7: Effect of Drug Substance on Sleep Cycle

The following example provides a study to evaluate the translational measure of functional brain architecture in rats following administration of the drug substance (DS) of Example 1. The objectives of the study were: i) to investigate the effects of acute administration of DS i.p. on EEG readouts; ii) to investigate whether acute administration of DS affect sleep architecture in rats with possible relevance to neuropsychiatric disorders; and iii) to investigate whether acute administration of DS produce changes in CNS spectral bands that could be interpreted as a unique signature/mechanism of action with translational potential.

Methods: vehicle only, 10 mg/kg, 30 mg/kg, 60 mg/kg, and 100 mg/kg of DS were intraperitoneally administered in male rats. Modafinil was used as a positive control. Rats were dosed once per week in a latin square design. Rats underwent surgery to implant 2 recording electrodes, the positive EEG electrode was attached to the anterior craniotomy and the negative EEG electrode to the posterior craniotomy.

Results: DS administration decreased gamma power in wake state in a dose response manner. Following DS administration, beta and gamma power were decreased in non-REM sleep; delta, theta, alpha, and beta power were increased in REM sleep; and gamma power was decreased in REM sleep. In addition, DS marginally increased NREM sleep and decreased REM sleep with no change in wake time. The results from this study suggest that managing sleep cycle by DS administration can be used for treating neurodegenerative disorders described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1. A drug substance, comprising:
about 65% w/w to about 95% w/w of cannabidiol (CBD) based on the total weight of the drug substance; and
one or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

2. The drug substance according to claim 1, wherein the drug substance comprises about 0.4% w/w to about 3.0% w/w trans-THC.

3. The drug substance of claim 1, wherein the drug substance comprises about 1.0% w/w to about 4.0% w/w CBC.

4. The drug substance of claim 1, wherein the drug substance comprises about 0.2% w/w to about 4.0% w/w CBG.

5. The drug substance of claim 1, wherein the drug substance comprises about 0.2% w/w to about 1.4% w/w CBDV.

6. The drug substance of claim 1, wherein the drug substance comprises about 0.1% w/w to about 0.8% w/w CBD-C4.

7. The drug substance of claim 1, wherein the drug substance comprises about 0.1% w/w to about 1.6% w/w cis-THC.

8. The drug substance of claim 1, wherein the drug substance further comprises one or more terpenes, triglycerides, sterols, fatty acids, or carotenoids.

9. The drug substance according to claim 8, wherein the drug substance comprises about 1.0% w/w to about 3.0% w/w terpenes.

10. The drug substance of claim 8, wherein the drug substance comprises about 0.8% w/w and 3.0% w/w triglycerides.

11. The drug substance of claim 8, wherein the drug substance comprises about 0.5% w/w to about 2.0% w/w sterols.

12. The drug substance according to claim 1, wherein the drug substance comprises two or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

13. The drug substance according to claim 1, wherein the drug substance comprises three or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

14. The drug substance according to claim 1, wherein the drug substance comprises four or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

15. The drug substance according to claim 1, wherein the drug substance comprises five or more of about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), or about 0.1% w/w to about 5.0% w/w of cis-tetrahydrocannabinol (cis-THC).

16. The drug substance according to claim 1, wherein the drug substance comprises about 0.1% w/w to about 5.0% w/w of trans-tetrahydrocannabinol (trans-THC), about 0.1% w/w to about 5.0% w/w of cannabichromene (CBC), about 0.1% w/w to about 5.0% w/w of cannabigerol (CBG), about 0.1% w/w to about 5.0% w/w of cannabidivarin (CBDV), about 0.1% w/w to about 5.0% w/w of cannabidiol-C4 (CBD-C4), and about 0.1% w/w to about 5.0% w/w of cis-tetrahydrocannabinol (cis-THC).

17. A pharmaceutical composition, comprising the drug substance of claim 1 and a pharmaceutically acceptable excipient.

18. The pharmaceutical composition according to claim 17, wherein the pharmaceutically acceptable excipient is a carrier, diluent, adjuvant, filler, buffer, binder, disintegrant, preservatives, antioxidant, lubricant, stabiliser, solubilizer, surfactant, masking agent, colouring agent, flavouring agent, or sweetening agent.

19. The pharmaceutical composition of claim 17, wherein the pharmaceutical composition is in a form of a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, or an aerosol.

20. A method of treating a neurological or neurodevelopmental disorder in a patient in need thereof, comprising administering the drug substance of claim 1.

21. The method of claim 20, wherein the neurological disorder or neurodevelopmental disorder is autism spectrum disorder (ASD), Alzheimer's Disease (AD), or psychiatric disorder.

22. The method of claim 21, wherein the psychiatric disorder is psychosis, depression, or schizophrenia.

23. A method of reducing symptoms associated with a neurological or neurodevelopmental disorder in a patient in need thereof, comprising administering the drug substance of claim 1.

24. The method of claim 23, wherein the symptoms comprise deficits in social interaction, agitation, anxiety, repetitive behaviours, irritability, aggression, hyperactivity, or a combination thereof.

25. A method of treating essential tremor in a patient in need thereof, comprising administering the drug substance of claim 1.
